(19) United States
(12) Patent Application Publication
(10) Pub. No.: US 2026/0144819 A1
(43) Pub. Date: May 28, 2026

(54) NK-92 Cells with Cytokine Induced Memory-Like Cytotoxicity And Derivatives Thereof

(71) Applicant: ImmunityBio, Inc., San Diego, CA (US)

(72) Inventors: Hans G. Klingemann, San Diego, CA (US); Laurent H. Boissel, San Diego, CA (US); Barry J. Simon, San Diego, CA (US); Patrick Soon-Shiong, Culver City, CA (US); Dustin Elwood, Derry, NH (US)

(73) Assignee: ImmunityBio, Inc., San Diego, CA (US)

(21) Appl. No.: 19/399,287

(22) Filed: Nov. 24, 2025

Related U.S. Application Data
(60) Provisional application No. 63/724,858, filed on Nov. 25, 2024.

(51) Int. Cl. A61K 35/17; A61K 40/15; A61K 40/31; A61K 40/42; A61P 35/00; C07K 14/54; C07K 14/735; C12N 5/00; C12N 5/0783

(52) U.S. Cl. CPC A61K 35/17; A61K 40/15; A61K 40/31; A61K 40/421; A61K 40/4234; A61P 35/00; C07K 14/5434; C07K 14/70535; C12N 5/0018; C12N 5/0646; C12N 2501/2312; C12N 2501/2315; C12N 2501/2318

(57) ABSTRACT

Methods and compositions are provided for generating cytokine-induced memory-like (CIML) phenotypes in NK-92-derived cell lines including activated NK (aNK) and PD-L1 chimeric antigen receptor NK (PD-L1 t-haNK) cells. Brief exposure of the cells to interleukin-12, interleukin-18, and the interleukin-15 (or interleukin-15 superagonist N-803) induces durable interferon-gamma expression and a memory recall response that persists for at least a week after stimulation. The disclosure includes methods for manufacturing, irradiating, and cryopreserving CIML aNK and CIML t-haNK cell products that provide a standardized off-the-shelf therapy for the targeted treatment of cancers, including tumors that are initially resistant to NK-cell cytotoxicity.

This application claims priority to US provisional application with the Ser. No. 63/724,858, filed Nov. 25, 2024, which is incorporated by reference herein in its entirety.

### FIELD OF THE INVENTION

The field of the invention is pharmaceutical compositions comprising natural killer cells, and especially cytokine-induced memory-like NK-92 cells and derivatives thereof as well as methods of production and their use.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Cytokine-induced memory-like (CIML) NK cells from blood, cord blood, or stem cells display some characteristics of adaptive immune memory T-cells, such as long-term persistence in vivo and enhanced effector functions. Such CIML-NK cells have first been described in mice where they were produced by short-term in vitro activation using two or more of IL-12, IL-15, and IL-18. In human, CIML NK cells from blood, cord blood, or stem cells they are typically produced by short-term activation with a cocktail of IL-12/IL-15/IL-18 cytokines. The activation confers durable changes in the NK cells behavior, with enhanced cytotoxic abilities and secretion of IFNγ, expansion and long-term persistence in vivo, as well as memory recall properties that lead to increased production of IFNγ upon later re-stimulation with cytokines or target cells. The properties of such CIML-NK cells make them desirable for use as therapy against cancer since their expansion and persistence in vivo allows for smaller infusion doses and eliminates the need for repeat dosing. Since fewer cells are needed overall, time and costs of cell expansion and production are notably reduced compared with other NK-cell therapies. Moreover, the anti-cancer activity of such CIML-NK cells can be further enhanced by expression of chimeric antigen receptors (CARs) or knockout of internal immune checkpoints such as CISH.

Most typically, such CIML-NK cells are produced from primary NK cells (peripheral or umbilical cord blood) or from iPSC-derived NK cells. Unfortunately, the former often suffer from initial blood product quality and donor-to-donor variability, while the latter requires a costly and complicated process. Moreover, while such CIML-NK cells do not cause GvHD in an allogeneic setting, they can still be rejected by the host's immune system. Prevention of immune rejection necessitates cumbersome engineering that can introduce batch-to-batch variability. In addition, IFNγ is known to upregulate expression of MHC class I and PD-L1 molecules on the surface of cells and enhanced secretion of IFNγ by such CIML-NK cells can potentially make cancer cells and tumor-associated cells such as MDSCs more resistant to killing by both NK and CD8+ T-cells.

For example, in one known approach, CIML-NK cells are generated by short-term exposure of primary NK cells to IL-12/IL-15/IL-18 cytokines. Several modalities exist to obtain enough CIML-NK cells for therapy. One is to perform the cytokine exposure on NK-cells isolated from fresh leukapheresis product, re-infuse them as soon as the exposure is completed, and give some supporting cytokine to the patient to drive CIML-NK expansion in vivo rather than in vitro. Such an approach has the advantage of minimal processing steps, however, is prone to large patient-to-patient variability, both for donor source material and in vivo expansion.

In another known approach, primary NK cells are expand in vitro either before or after cytokine exposure. Although such approach offers more control on the expansion stage, that approach is yet again prone to donor-to-donor variability and is often dependent on the quality of the source material. Yet another known approach is to use iPSC-derived NK cells. Advantageously, such approach has the advantage of using a clonal, characterized source and controlled expansion. Unfortunately, such approach is costly and cumbersome. Moreover, in addition to the effect of donor-to-donor variability and differences in source quality on cell expansion, the same factors may affect the level of expression of various factors, and especially IFNγ in such CIML-NK cells. Exemplary protocols to generate primary CIML-NK cells are disclosed in US 2024/0228964, WO 2022/187207, and WO 2021/006876.

Primary NK-cells or primary or stem-cell derived CIML-NK cells can also be engineered to make them express a PD-L1 CAR, either through stable transduction or transient transfection. However, such approach can be technically challenging and introduces batch-to-batch variability. In an attempt to remedy such defects, clones of engineered iPSC cells can be selected and banked prior to NK cell differentiation, which limits batch-to-batch variability. However, the differentiation process nevertheless remains cumbersome and costly. Moreover, engineering of autologous primary NK-cells or primary CIML-NK cells takes significant time between isolation from the primary donor/patient and final patient delivery due to expansion, quality control, and/or banking.

Alternatively, targeting of PD-L1 expressing cancer cells via an anti-PD-L1 antibody and ADCC can be achieved, but is dependent of the expression of CD16 on the effector cells, which is notoriously unpredictable and often varies considerably depending on the donor and the source materials.

Immortalized NK cell lines such as NK-92 (commercially available) have a distinct advantage over primary NK-cells in that they are homogenous, can be expanded to unlimited numbers in vitro, and can easily be engineered to express CARs or for gene knockout. However, immortal NK cell lines are generally considered unlikely to be sensitive to memory-like conversion (see e.g., Pal et al. Oncoimmunology 2017).

Thus, even though various compositions and methods of generating CIML NK cells are known in the art, all or almost all of them suffer from several drawbacks. Among other problems, currently known CIML-NK cells suffer from significant donor-to-donor variability, reliable behavior after cytokine stimulation, challenges with cell expansion, and/or high levels of constitutively secreted IFNγ. Therefore, there remains a need for improved systems and methods for improved compositions and methods to generate CIML NK cells.

### SUMMARY OF THE INVENTION

The inventive subject matter is directed to various compositions and methods of CIML aNK cells and derivatives thereof having extended memory recall features and superior, and in some cases synergistic cytotoxic activity.

In one aspect, the inventive subject matter relates to methods and compositions for generating and using cytokine-induced memory-like (CIML) NK-92 cells for the treatment of cancer. More particularly, the disclosure provides methods of exposing NK-92 cells to defined cytokine cocktails to induce durable memory-like properties, enhanced cytokine secretion, and anti-tumor activity, as well as methods of administering such CIML NK-92 cells, including genetically engineered derivatives, as therapeutic agents.

In one aspect, the disclosure provides a method of treating a patient with cancer, comprising exposing NK-92 cells to a cytokine cocktail comprising IL-12, IL-15 or an IL-15 superagonist such as N-803, and IL-18 (or fusion proteins comprising the same), for a period sufficient to induce CIML-like properties, removing the cytokine cocktail, and administering a therapeutically effective amount of the NK-92 cells to the patient. The cytokine-exposed NK-92 cells exhibit enhanced secretion of interferon-gamma (IFNγ), sustained expression of activation markers, and memory recall behavior such that upon subsequent exposure to target cells or cytokines, the NK-92 cells displayed increased cytotoxic activity and cytokine production.

The CIML-like properties induced by the cytokine treatment can persist for multiple days post-treatment, providing enhanced in vivo persistence, memory recall, and anti-tumor activity. The NK-92 cells can be administered immediately after cytokine treatment or after cryopreservation, providing a flexible and off-the-shelf therapeutic approach.

In certain embodiments, the NK-92 cells are genetically engineered to express recombinant IL-2 (erIL-2) and CD16, or to express a chimeric antigen receptor (CAR) with specificity for tumor or viral antigens, including but not limited to PD-L1, EGFR, HER2, CD19, B7-H4, or BCMA. Such CAR-engineered CIML NK-92 cells exhibit enhanced cytotoxicity against target cells, which may be further amplified via a positive feedback mechanism in which IFNγ secreted by the NK-92 cells induces upregulation of PD-L1 on target cells, thereby increasing CAR-mediated targeting and killing.

The disclosure further provides compositions comprising cytokine-induced NK-92 cells or their derivatives, including irradiated, cryopreserved, or genetically engineered variants, that retain memory-like behavior and enhanced cytokine secretion. These compositions can be used in methods for killing target cells, treating cancer, or modulating the tumor microenvironment, including reducing immunosuppressive cells such as myeloid-derived suppressor cells (MDSCs). Progeny cells of the cytokine-induced NK-92 cells also retain these enhanced properties, providing a renewable source of effector cells.

Overall, the disclosure provides a novel and practical approach to generating NK-92 cells with CIML-like properties, enabling effective, persistent, and memory-capable cellular immunotherapies for cancer. The methods and compositions disclosed herein offer significant advantages over conventional NK cell therapies, including reduced cell dosing requirements, enhanced in vivo persistence, off-the-shelf availability, and synergistic activity when combined with CAR engineering.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 depicts exemplary results for expression of surface markers on cytokine treated aNK cells. Small increase was seen in CD25, no change in TIGIT, NKp46, NKp44, CD62L, NKG2A, or NKG2D.

FIG. 2 depicts exemplary results for production of granzyme and IFN-γ secretion from cytokine treated aNK cells. CIML-stimulated aNK cells strongly upregulated IFNγ production, and moderately increased Granzyme B production.

FIG. 3A and FIG. 3B depicts exemplary results for the memory for production of granzyme and IFN-γ secretion from cytokine treated aNK cells and PD-L1 t-haNK cells. Increased expression of intracellular IFNγ was detected for 7 days post CIML-stimulation.

FIG. 4 depicts exemplary results for durable IFN-γ “memory recall” secretion from cytokine cocktail treated aNK cells upon target cell contact.

FIG. 5 depicts exemplary results for memory recall as measured by IFN-γ secretion from cytokine treated aNK cells upon target cell contact.

FIG. 6 depicts exemplary results that M-aNK supernatant enhances upregulation of PD-L1 expression on target cells THP-1 and SK-ES-1.

FIG. 7A and FIG. 7B depict exemplary results for in vitro cytotoxicity assays on target cells that were supernatant-treated using aNK or PD-L1 t-haNK cells as effectors. Supernatant from CIML-treated aNK and PD-L1 t-haNK cells was found to make THP-1 and SK-ES-1 target cells highly and selectively sensitive to killing by PD-L1 t-haNK effector cells.

FIG. 8A provides a flowchart and FIG. 8B provides a table comprising laboratory execution of the CIML induction and sampling protocol of aNK cells.

FIG. 9 provides exemplary experimental workflow and timelines for cytokine stimulation, irradiation, and sampling.

FIG. 10 provides exemplary experimental workflow and timelines for cytokine stimulation, irradiation, and sampling.

FIG. 11 depicts exemplary results for expression of surface markers on cytokine cocktail treated aNK cells (M-aNK cells).

FIG. 12 depicts exemplary results for cytotoxicity assays on cytokine cocktail treated aNK cells (M-aNK cells).

FIG. 13 depicts exemplary supernatant volumes and timing (before and after cGM exchange) for cytokine cocktail treated aNK cells (M-aNK cells).

FIGS. 14-17 depict exemplary describe phenotypic profiling by mass cytometry, measuring key NK surface and intracellular markers for cytokine cocktail treated aNK cells (M-aNK cells).

FIG. 18 provides a flowchart comprising laboratory execution of the CIML induction and sampling protocol of PD-L1 t-haNK cells.

FIG. 19 provides a table comprising laboratory execution of the CIML induction and sampling protocol of PD-L1 t-haNK cells.

FIG. 20 provides exemplary experimental workflow and timelines for cytokine stimulation, irradiation, and sampling of PD-L1 t-haNK cells.

FIG. 21 provides exemplary experimental workflow and timelines for cytokine stimulation, irradiation, and sampling of PD-L1 t-haNK cells.

FIG. 22 depicts exemplary results for expression of surface markers on cytokine cocktail treated PD-L1 t-haNK cells (M-PD-L1 t-haNK cells).

FIG. 23 depicts exemplary results for PD-L1 CAR expression on cytokine cocktail treated PD-L1 t-haNK cells (M-PD-L1 t-haNK cells).

FIG. 24 depicts exemplary results for cytotoxicity assays on cytokine cocktail treated PD-L1 t-haNK cells (M-PD-L1 t-haNK cells).

FIG. 25 depicts exemplary supernatant volumes and timing (before and after cGM exchange) for cytokine cocktail treated PD-L1 t-haNK cells (M-PD-L1 t-haNK cells).

FIGS. 26-29 depict exemplary describe phenotypic profiling by mass cytometry, measuring key NK surface and intracellular markers for cytokine cocktail treated PD-L1 t-haNK cells (M-PD-L1 t-haNK cells).

### DETAILED DESCRIPTION

The inventors have now unexpectedly discovered that CIML NK cells can be produced from an immortalized NK cell line, and especially from NK-92 (aNK) cells and derivatives thereof, that have desirable cytotoxic activity with extended recall memory behavior. Most advantageously, and unexpectedly, the cells presented/contemplated herein exhibited memory recall over a period of at least 7 days, and more typically over at least 14 days, even where the cells were no longer subject to cytokine stimulation. Therefore, in still further observed aspects, the memory recall also extended to progeny cells of the stimulated cells, and was even observed with progeny cells (e.g., up to 3^{rd}, 4^{th}, or 5^{th} generation) expressing or displaying an antigen-specific chimeric antigen receptor (CAR).

CIML NK cells have garnered a lot of attention as cellular therapeutic agents due to their much-increased lifespan in patients and their enhanced memory response upon re-challenge by cytokines or target cells. A key part of this memory recall feature is the increased secretion of interferon gamma (IFNγ). This potent immune stimulator can trigger upregulation of MHC-I molecules expression on the surface of tumor cells, thereby pushing the tumor towards an immunologically “hot” status.

Cytokine-induced memory-like (CIML) features, such as durable IFNγ expression and cytokine recall, had been documented only in primary NK cells from donors, not in immortalized NK-92-derived cell lines. NK-92 cells are highly engineered and continuously cultured, and therefore they were believed to lack the epigenetic flexibility required for “training” or memory formation. However, the present inventors have surprisingly found that a short, overnight stimulation with IL-12, IL-18, and IL-15 (or N-803) reprogrammed NK-92 (aNK) and t-haNK cells to exhibit long-lasting IFNγ induction and memory recall upon re-challenge, which are behaviors previously thought unique to primary NKs. This establishes, for the first time, that memory-like reprogramming is possible in an immortalized therapeutic NK line, overturning conventional expectations in NK cell biology.

The inventors have now discovered that similarly to primary NK cells, the natural killer cell line NK-92 (aNK) can acquire a memory-like phenotype upon (e.g., overnight) induction with IL-12, IL-18, and the IL-15 superagonist N-803 (or IL-15), as evidenced by increased steady-state IFNγ secretion (typically >50-fold increase) and up-regulation of CD25. Most notably, the inventors recognized that although the initial increase in IFNγ secretion subsided after about 48 hrs, the so generated memory-like aNK cells displayed a memory recall feature for several days post-activation, as evidenced by enhanced IFNγ response upon challenge with K562, Raji, and THP-1 cells compared to non-memory-like cells (2.3, 6.9, and 3.8-fold increases after 1 week, respectively).

The durable IFNγ expression without continuous cytokine support is unexpected because typical NK-92 lines require constant exogenous IL-2 or IL-15 for IFNγ secretion. Removal of cytokines usually causes rapid decline in function within 24-48 hours. However, the inventors have shown herein a surprising result that following just 16 h of cytokine induction, aNK and PD-L1 t-haNK maintained 6- to 8-fold higher IFNγ secretion for at least seven days, even under normal IL-2 culture conditions. This demonstrates intrinsic transcriptional memory and persistence of effector programming, contrary to prior expectations for IL-2-dependent NK cell lines.

Moreover, NK-92 cells expressing a high-affinity CD16 and engineered to express an anti-PD-L1 CAR (PD-L1 t-haNK) maintained their susceptibility to memory-like cytokines induction. As shown in more detail below, exposure of the NK-resistant cells lines THP-1 and SK-ES-1 to culture supernatant from memory-like PD-L1 t-haNK triggered expression of PD-L1 on their surface, which in turn made them highly sensitive to PD-L1 CAR-mediated targeted killing by the PD-L1 t-haNK cells in a 4 hours in vitro cytotoxicity assay (respectively 91% and 88% killing at E:T ratio of 1.25:1, compared to 54% and 41% for target cells exposed to regular PD-L1 t-haNK supernatant), while treatment with the supernatant did not affect their resistance to NK-92 cells. This is highly unexpected because expression of chimeric antigen receptors often alters downstream signaling (e.g., tonic activation), which can interfere with cytokine responsiveness or cause exhaustion. However, on the contrary, the inventors surprisingly found that PD-L1 t-haNK cells responded to the cytokine cocktail identically to unmodified aNK cells, maintaining full CAR expression, IFNγ induction, and recall capacity. Thus, CAR engineering does not impair memory-like differentiation, enabling a new class of CAR-CIML NK-92 products.

It should be particularly appreciated that infusion of a cellular therapy product that constitutively secretes high IFNγ may cause detrimental side effects. For example, IFNγ also triggers upregulation of the immune checkpoint molecule PD-L1. Conventional thinking predicts that PD-L1 upregulation protects tumors by engaging PD-1 on T cells, which is an immune escape mechanism. However, the inventors surprisingly found that in the presently disclosed methods and compositions, PD-L1 induction actually sensitizes tumors to PD-L1 CAR-mediated NK killing. When exposed to supernatant from Memory-aNK or Memory-PD-L1 t-haNK cells, previously NK-resistant lines (THP-1, SK-ES-1) became highly susceptible (41→88% killing). Thus, an ordinarily immune-evasive mechanism (PD-L1 upregulation) is repurposed into a vulnerability, creating a positive feedback loop: →IFNγ secretion→PD-L1 induction→enhanced PD-L1 CAR recognition→further IFNγ release. This reversal of immune escape is biologically counterintuitive and highly surprising. In other words, the memory-like PD-L1 t-haNK cells presented herein advantageously uses their (extended) memory recall ability and their ability to deliver substantial quantities of IFNγ upon locally engaging with tumor cells specifically at the tumor site. This increase then drives upregulation of PD-L1 expression on neighboring tumor cells, creating an expanding wave of targets sensitized to killing by PD-L1 t-haNK cells.

In still further contemplated aspects, memory-like aNK cell line or memory-like PD-L1 t-haNK cell line is equipped with inducible safety switch features (e.g., inducible suicide genes). Such a feature would remove the need for irradiation and to extend their half-life in patients. Such and other cells contemplated herein will therefore constitute a promising “off-the-shelf” cellular therapy against immunologically cold cancers.

The term “suicide gene” refers to expression of a transgene that allows for the negative selection of cells expressing the suicide gene. A suicide gene is used as a safety system, allowing cells expressing the gene to be killed by introduction of a selective agent. This is desirable in case the recombinant gene causes a mutation leading to uncontrolled cell growth, or the cells themselves are capable of such growth. A number of suicide gene systems have been identified, including the herpes simplex virus thymidine kinase (TK) gene, the cytosine deaminase gene, the varicella-zoster virus thymidine kinase gene, the nitroreductase gene, the Escherichia coli gpt gene, and the E. coli Deo gene. Typically, the suicide gene encodes for a protein that has no ill effect on the cell but, in the presence of a specific compound, will kill the cell. Thus, the suicide gene is typically part of a system.

In one embodiment, the suicide gene is active in NK-92 cells. In one embodiment, the suicide gene is the thymidine kinase (TK) gene. The TK gene may be a wild-type or mutant TK gene (e.g., tk30, tk75, sr39tk). Cells expressing the TK protein can be killed using ganciclovir. In another embodiment, the suicide gene is cytosine deaminase, which is toxic to cells in the presence of 5-fluorocytosine. Garcia-Sanchez et al. “Cytosine deaminase adenoviral vector and 5-fluorocytosine selectively reduce breast cancer cells 1 million-fold when they contaminate hematopoietic cells: a potential purging method for autologous transplantation.” Blood. 1998 Jul. 15; 92(2):672-82. In a further embodiment, the suicide gene is cytochrome P450, which is toxic in the presence of ifosfamide or cyclophosphamide. See, e.g. Touati et al. “A suicide gene therapy combining the improvement of cyclophosphamide tumor cytotoxicity and the development of an anti-tumor immune response.” Curr Gene Ther. 2014; 14(3):236-46. In yet another embodiment, the suicide gene is iCasp9. Di Stasi, (2011) “Inducible apoptosis as a safety switch for adoptive cell therapy.” N Engl J Med 365: 1673-1683. See also Morgan, “Live and Let Die: A New Suicide Gene Therapy Moves to the Clinic” Molecular Therapy (2012); 20: 11-13. iCasp9 induces apoptosis in the presence of a small molecule, AP1903. AP1903 is biologically inert small molecule, that has been shown in clinical studies to be well tolerated and has been used in the context of adoptive cell therapy.

As can be taken from the data presented herein, upon short-term stimulation by a cocktail of IL-12, IL-18, and IL-15 (or N-803) cytokines, NK-92 cells acquire CIML-like properties such as increased secretion of IFNγ and memory recall properties, with the recall properties remarkably lasting for more than one week, and even two weeks or more post-stimulation. Moreover, it was also observed that where the aNK cell or haNK cell expressed a CAR (e.g., PD-L1 CAR-expressing aNK or haNK cells), these cells were susceptible to the same cytokine stimulation and their increased IFNγ secretion triggered a positive feedback loop where stimulation of PD-L1 expression on target cells by IFNγ leads to increased targeting by PD-L1 CAR, which facilitates enhanced killing and further stimulation of effector cells to secrete IFNγ. It is further contemplated that such a feedback loop may not be limited to cancer targets cells but may also affect the tumor microenvironment, as MDSCs can also be eliminated by PD-L1 t-haNK cells. Furthermore, it should be appreciated that because the so treated aNK, haNK, and t-haNK cells display a prolonged recall memory behavior, the thusly treated cells can be cryopreserved and can therefore become available as an off-the shelf product.

Viewed form a different perspective, it should therefore be appreciated that using the compositions and methods presented herein clonal, well-characterized cell lines with defined growth conditions can be prepared that exhibit no donor-to-donor variability, and that exhibit reliable behavior after cytokine treatment. In addition, and in part because the contemplated cells as based on aNK cells (NK-92 cells), the calls can be readily further genetically engineered to acquire additional properties (e.g., expression of high affinity CD16 receptor, expression of a CAR (which may be cancer or tumor associated antigen specific, or that may bind to a tumor neoepitope)). Alternatively, existing engineered cells lines based on NK-92 cells (e.g., clonal PD-L1 t-haNK cell line) are also deemed suitable for use herein. Consequently, it should be recognized that aNK, PD-L1 and other CAR-expressing aNK cells or haNK cells, or still other aNK variants (e.g., modified or unmodified NK-92 cells expressing IL-15) allows expansion, cytokine/drug combination treatment, quality control, and banking in advance, thereby creating an “off-the-shelf” product. As a result, patients can receive treatment in a more timely and immediate manner. As will be further recognized, the CIML aNK cells and derivatives thereof (e.g., haNK cells, t-haNK cells, etc.) will typically be irradiated to a level to abrogate cell propagation (e.g., about 10 Gy) and/or cryopreserved for extended storage (e.g., in vapor phase of liquid nitrogen, or at or below −78° C.).

Moreover, it should be noted that any cell preparations post-cytokine stimulation will typically be transfused in a medium that is substantially free of cytokines (e.g., cryopreservation medium with or without human albumin) to so avoid any side effects due to the presence of cytokines in the transfusion medium. As such, it should also be recognized that a significant fraction of the transfused cells may also be 2^{nd}, 3^{rd}, 4^{th}, and/or 5^{th} generation (and even higher) progeny cells of the originally stimulated NK-92 cells (or derivatives thereof). Most notably, and quite unexpectedly, the enhanced memory recall function in the progeny cells is ‘inherited’ from the stimulated parental cells. Likewise, the targeting capability of the progeny cells via an expressed or displayed CAR was also observed in the progeny cells.

Throughout the present disclosure, the term “NK-92” refers to a cell line that is a continuously growing, interleukin-2 (IL-2)-dependent natural killer (NK) cell line originally established from a patient with NK-cell lymphoma. These cells retain the core phenotypic and functional characteristics of activated NK cells, including cytotoxic activity, expression of activating and inhibitory NK receptors, and responsiveness to cytokine stimulation. In therapeutic and research contexts, NK-92 cells and their clinically optimized derivatives are often referred to as activated NK cells or aNK cells, to emphasize their constitutively activated phenotype and cytokine dependence. Thus, as used herein, the terms “NK-92 cells” and “aNK cells” are used interchangeably to refer to this established, IL-2-maintained NK-92-derived cell line utilized as a platform for genetic modification and immunotherapy development.

As used herein, the term haNK (high-affinity NK) refers to an NK-92-derived cell line genetically engineered to express a high-affinity, non-cleavable variant of the Fc receptor CD16 (FcγRIIIa, V158), enabling efficient antibody-dependent cellular cytotoxicity (ADCC) in combination with therapeutic monoclonal antibodies. The t-haNK (targeted haNK) lineage represents a further engineered form of haNK cells that express a chimeric antigen receptor (CAR) conferring direct antigen-specific recognition of tumor targets. For example, PD-L1 t-haNK cells express a CAR recognizing the immune checkpoint ligand PD-L1. Collectively, these NK-92-derived cell lines (aNK, haNK, and t-haNK) constitute an integrated cellular immunotherapy platform capable of combining innate NK cytotoxicity, antibody-dependent killing, and CAR-mediated antigen specificity within a standardized, irradiated “off-the-shelf” allogeneic product.

In one embodiment, the IL-2 is cloned and expressed with a signal sequence that directs the IL-2 to the endoplasmic reticulum (erIL-2). This permits expression of IL-2 at levels sufficient for autocrine activation, but without releasing IL-2 extracellularly. See Konstantinidis et al “Targeting IL-2 to the endoplasmic reticulum confines autocrine growth stimulation to NK-92™ cells” Exp Hematol. 2005 February; 33(2):159-64. Continuous activation of the FcR-expressing NK-92 cells can be prevented, e.g., by the presence of the suicide gene. erIL-2 is further described in WO2020/091868, which is incorporated by reference herein in its entirety.

In one aspect of the inventive subject matter, the inventors contemplate a method of killing target cells that includes a step of exposing a first plurality of target cells to PD-L1 CIML t-haNK cells in an environment to thereby kill at least some of the first plurality of target cells. In such method, a second plurality of the target cells is more susceptible to being killed by the PD-L1 CIML t-haNK cells (e.g., increase in susceptibility is at least 40%, or at least 80%).

Typically, the PD-L1 CIML t-haNK cells secrete IFNγ upon contact with the target cells in an amount that is at least 20-fold, or at least 40-fold as compared to unstimulated NK-92 cells. Moreover, it is contemplated that the PD-L1 CIML t-haNK cells comprise cytotoxic progeny PD-L1 CIML t-haNK cells. Where desired, the step of exposing is performed in a medium that does not include exogenous cytokines, and as such the step of exposing may be performed in vivo (e.g., tumor microenvironment).

In another aspect of the inventive subject matter, the inventors contemplate a method of triggering memory in a CIML aNK cell or derivative thereof that includes a step of contacting the CIML aNK cell or derivative thereof with a target cell, wherein the step of contacting triggers release of IFNγ in the CIML aNK cell or derivative thereof. Most typically, the release of IFNγ renders the target cells more susceptible to being killed by the CIML aNK cell. For example, the CIML aNK cell or derivative thereof is a PD-L1 CIML t-haNK cell. In further examples, the target cell is a NK resistant target cell, and/or a cell that expresses PD-L1 upon release of IFNγ.

Therefore, the inventors also contemplate a composition comprising cytokine-induced NK-92 cells or derivatives thereof having enhanced cytokine secretion that is at least 40-fold more than unstimulated NK-92 cells, after initial cytokine induction/treatment. Typically, expression of TIGIT, NKG2A, NKG2D, NKp44, and NKp64 in the cytokine-induced NK-92 cells or derivatives thereof remains unchanged upon and/or after cytokine stimulation. In some examples, the cytokine-induced NK-92 cells or derivatives thereof are cytokine-induced haNK cells or t-haNK cells, which may be irradiated and/or cryopreserved. Where desired, the cytokine-induced NK-92 cells or derivatives thereof may further comprise a recombinant nucleic acid that encodes a cytokine, a TGFβ trap, and/or a suicide gene.

Viewed from a different perspective, the inventors also contemplate a composition of cytokine-induced NK-92 cells or derivative thereof, wherein the cytokine-induced NK-92 cells or derivative thereof exhibit enhanced cytokine secretion at a subsequent challenge in the presence of cytokine or a target cell, and wherein the enhanced cytokine secretion is at least 3- to 10-fold as compared to an unstimulated NK-92 cell. For example, the enhanced cytokine secretion is between 7 and 14 days after initial induction. As desired, the cytokine-induced NK-92 cell or derivative is irradiated in an amount effective to abrogate proliferation. In further example, the cytokine-induced NK-92 cell or derivative may be a PD-L1 cytokine-induced t-haNK.

Additionally, and due to the extended memory recall of contemplated cells, it should be recognized that the inventors also contemplate a progeny cell of the cytokine-induced NK-92 cell or derivative thereof as described herein, having enhanced IFNγ secretion upon cytokine or target cell stimulation, wherein the progeny cell is at least a 2nd generation cell (e.g., 3rd or 4^{th} generation progeny cell).

In view of the above, the inventors therefore also contemplate a method of treatment for a patient with cancer, the method comprising a step of exposing NK-92 cells, or a genetically engineered derivative thereof, to a medium comprising a cytokine cocktail, wherein the cytokine cocktail comprises individual cytokines or fusion proteins comprised thereof, wherein the cocktail comprises IL-15 or N-803, IL-12, and IL-18; removing the cytokine cocktail from the medium; and administering a therapeutically effective amount of the NK-92 cells to the patient.

In some embodiments, the NK-92 cells are exposed to the cytokine cocktail for 12 hours, 24 hours, 36 hours, 48 hours, or 96 hours, and in other embodiments, the NK-92 cells are cryopreserved after removing the cytokine cocktail. Typically, but not necessarily, the NK-92 cells are administered 1 day, 2 days, 3 days, 4 days, 5 days, 6 days, 7 days, or more than 7 days after removing the cytokine cocktail. In further exemplary aspects, the genetically engineered NK-92 cells express erIL-2 and CD16, and/or a Chimeric Antigen Receptor (CAR). In such case, the CAR may comprise an extracellular binding domain having specific binding affinity for a tumor (e.g., PD-L1, EGFR, HER2, CD19, B7-H4, or BCMA) or viral antigen (e.g., gp120). In further embodiments, the cytokine cocktail comprises N-803 and/or HCW-9201. Thus, contemplated cytokine cocktails may comprise a fusion protein comprising an IL-12 moiety and an IL-18 moiety.

In still another aspect of the inventive subject matter, the inventors contemplate a method of generating memory-like NK-92 cells for use in the treatment of a patient with cancer, the method comprising the steps of exposing NK-92 cells, or a genetically engineered derivative thereof, to a medium comprising a cytokine cocktail, wherein the cytokine cocktail comprises individual cytokines or fusion proteins comprised thereof, wherein the cocktail comprises IL-15 or N-803, IL-12, and IL-18; removing the cytokine cocktail from the medium; and administering a therapeutically effective amount of the NK-92 cells to the patient.

Therefore, the inventors also contemplate a pharmaceutical composition for use in generating memory-like NK-92 cells for the treatment of a patient with cancer, the composition comprising: NK-92 cells, or a genetically engineered derivative thereof, and a medium comprising a cytokine cocktail, wherein the cytokine cocktail comprises individual cytokines or fusion proteins comprised thereof, wherein the cocktail comprises IL-15 or N-803, IL-12, and IL-18.

Viewed from a different perspective, the inventors also contemplate a method of stimulating interferon gamma (IFNγ) production in an NK-92 cell for use in the treatment of a patient with cancer, the method comprising: exposing NK-92 cells, or a genetically engineered derivative thereof, to a cytokine cocktail comprising individual cytokines or fusion proteins comprised thereof, wherein the cocktail comprises IL-15 activity, IL-12 activity, and IL-18 activity; removing the cytokine cocktail; and administering a therapeutically effective amount of the NK-92 cells to the patient. Most typically, the NK-92 cells release INFγ upon contact with a cancer cell, the cancer cell increases expression of MHC-I upon contact with the NK-92 cell, and/or the cancer cell increases expression of PD-L1 upon contact with the NK-92 cell.

Throughout the disclosure, the term N-803, also known as the IL-15:IL-15Rα complex, nogapendekin alfa-imbakicept (NAI), ALT-803, Anktiva, or IL-15N72D:1L-15RαSu/IgG1, refers to an IL-15-based immunostimulatory protein complex comprising two protein subunits of a human IL-15 variant associated with high affinity to a dimeric human IL-15 receptor α (IL-15Rα) sushi domain/human IgG1 Fc fusion protein (J Immunol (2009) 183: 3598-3607). The IL-15 variant is a 114 amino acid polypeptide comprising the mature human IL-15 cytokine sequence, with an asparagine to aspartate substitution at position 72 of helix C (N72D). The human IL-15Rα sushi domain/human IgG1 Fc fusion protein comprises the sushi domain of the human IL-15 receptor α subunit (IL-15Rα) (amino acids 1-65 of the mature human IL-15Rα protein) linked to the human IgG1 CH2-CH3 region containing the Fc domain (232 amino acids). U.S. Pat. No. 9,328,159, which describes NAI, is incorporated herein by reference in its entirety.

Most typically, the N-803 complex may have a molecular weight of approximately 114 kDa and will advantageously exhibit an extended serum half-life compared to native IL-15 due to the Fc fusion. Among further benefits, it should also be noted that N-803 may stimulate proliferation and/or activation of iNKT cells. Moreover, N-803 will also induce production of cytokines like IFNγ. Suitable variants of N-803 may include complexes with different IL-15 mutations, such as IL-15N72A. The Fc portion may be derived from other IgG subtypes. Linker sequences between the components may be modified. The ratio of IL-15 to IL-15Rα may be altered. Exemplary sequences for suitable N-803 and analogs are shown in SEQ ID NO:1-4, of U.S. Patent Application No. 63/768,448, which is incorporated by reference, and which forms an express part of this disclosure. Other suitable IL-15 or IL-15:IL-15Ra constructs may comprise the single chain constructs of U.S. Pat. No. 11,401,324, or the multi-chain constructs of U.S. Pat. No. 11,518,792.

### EXAMPLES

NK-92 (aNK) cells were treated overnight with the cytokines IL-2, IL-15, N-803, IL-12, IL-18, singly or in combinations. Specific exemplary conditions are shown in the Table 1 below.

| Conditions | Concentrations | |
|---|---|---|
| IL-2 only | 500 | IU/mL |
| N-803 only | 175 | ng/mL |
| N-803 cocktail | 10 + 175 + 50 | ng/mL |
| (IL-12 + N803 + IL18) | | |
| IL-15 cocktail | 10 + 50 + 50 | ng/mL |
| (IL-12 + IL15 + IL18) | | |

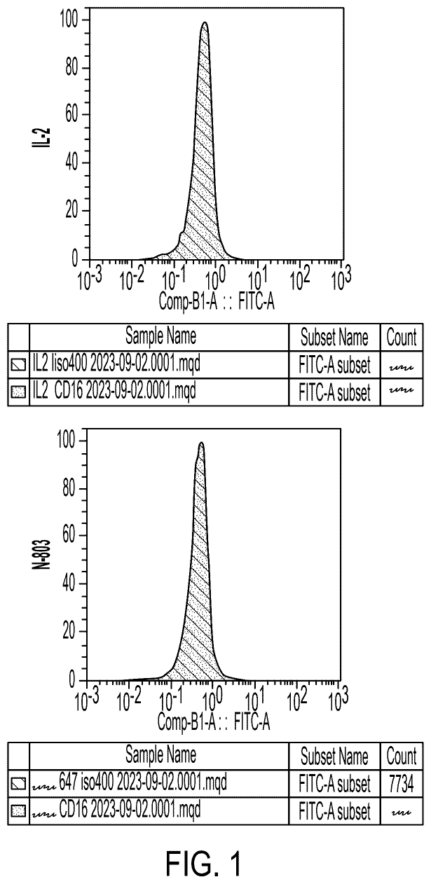

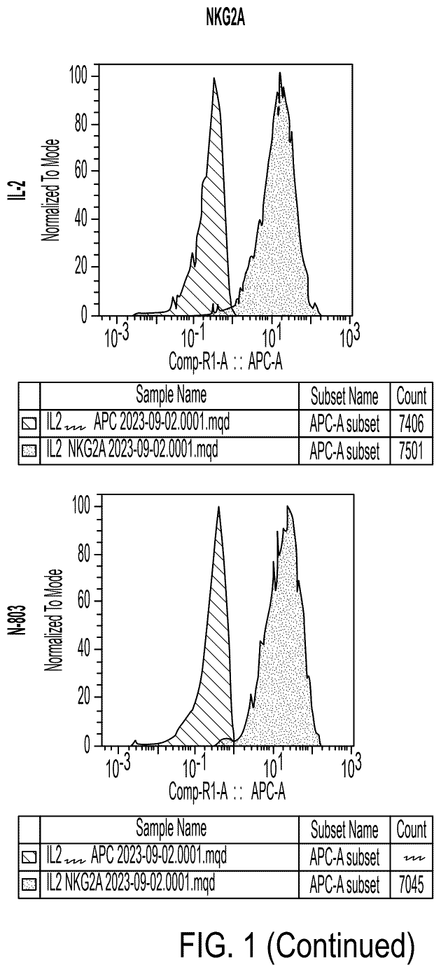

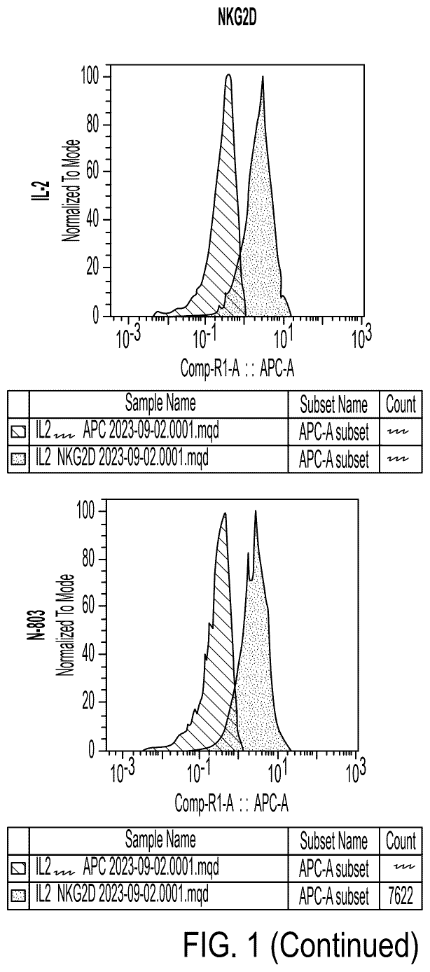

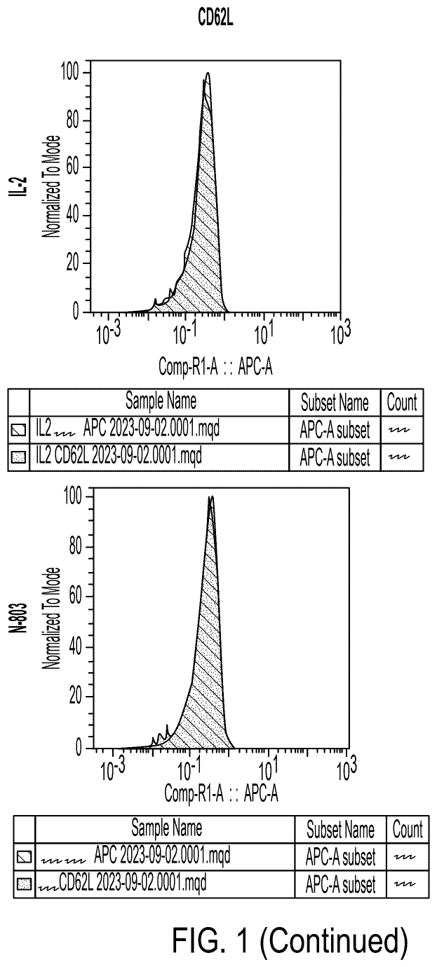

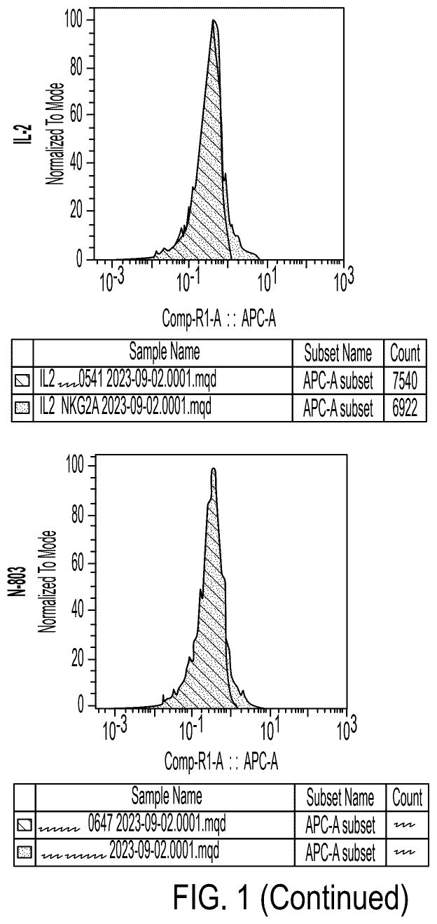

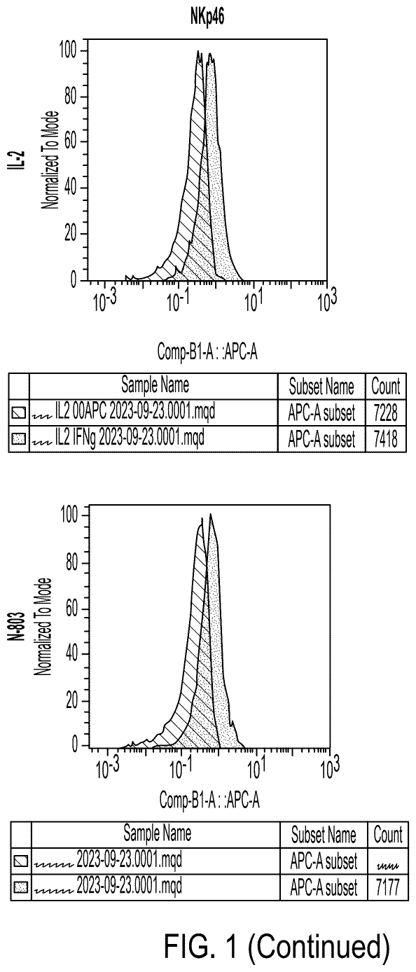

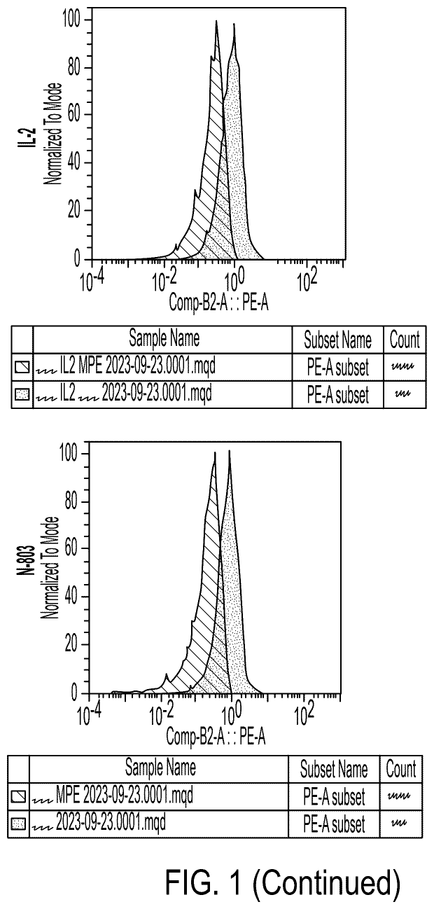

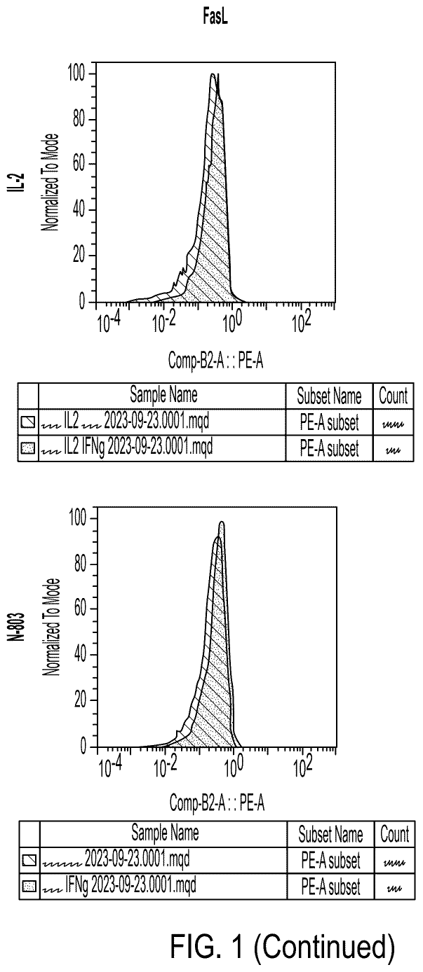

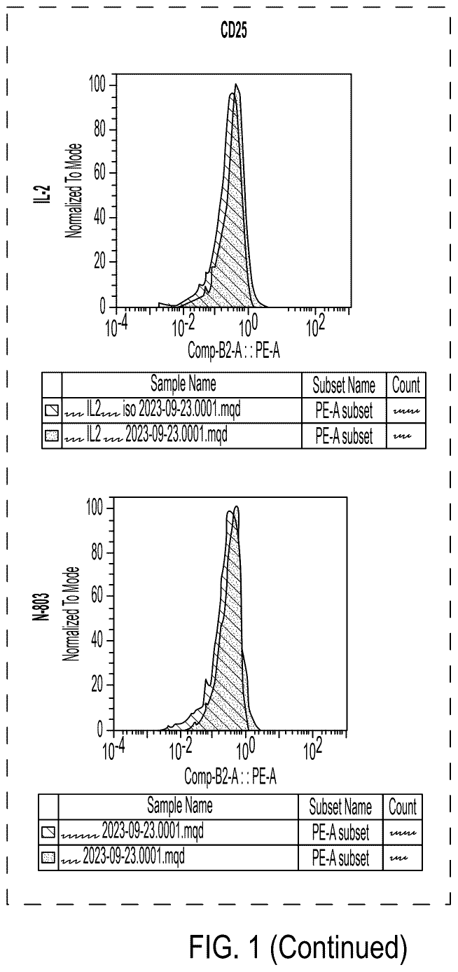

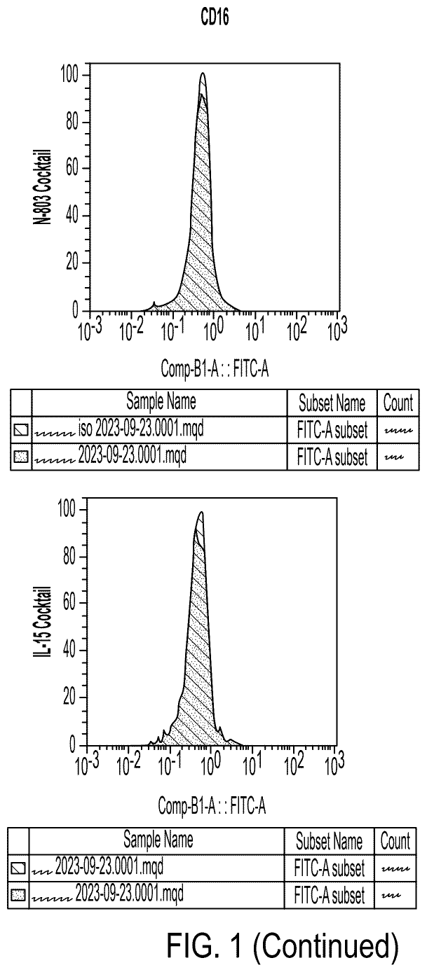

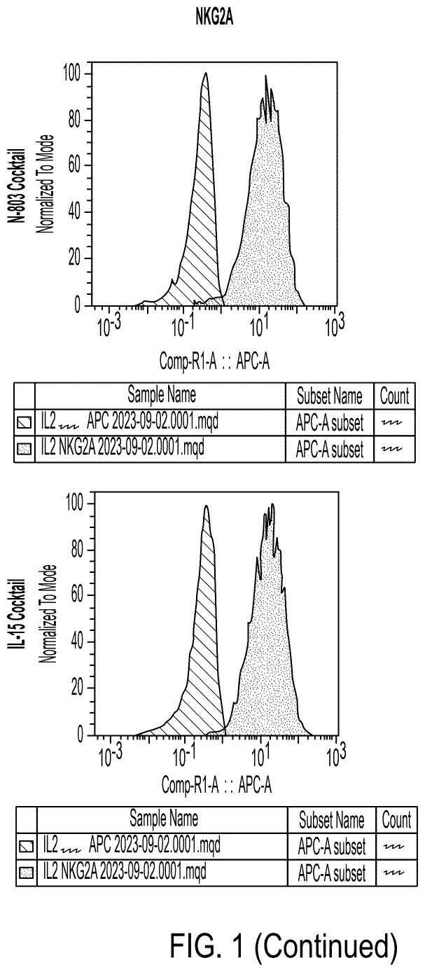

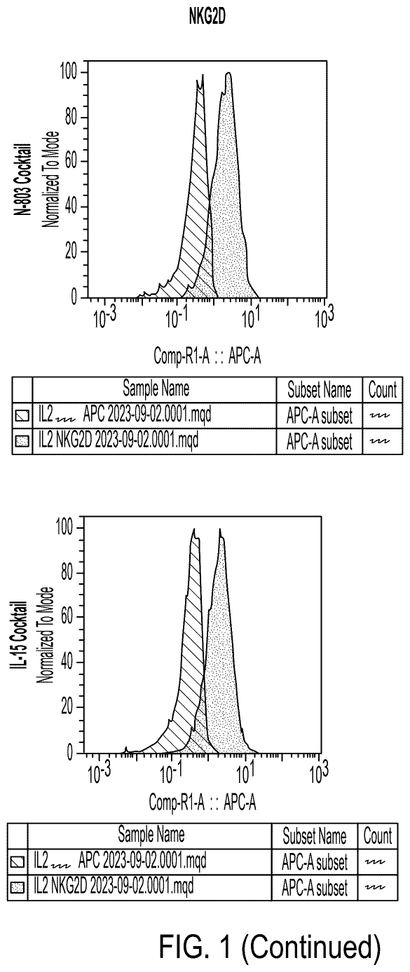

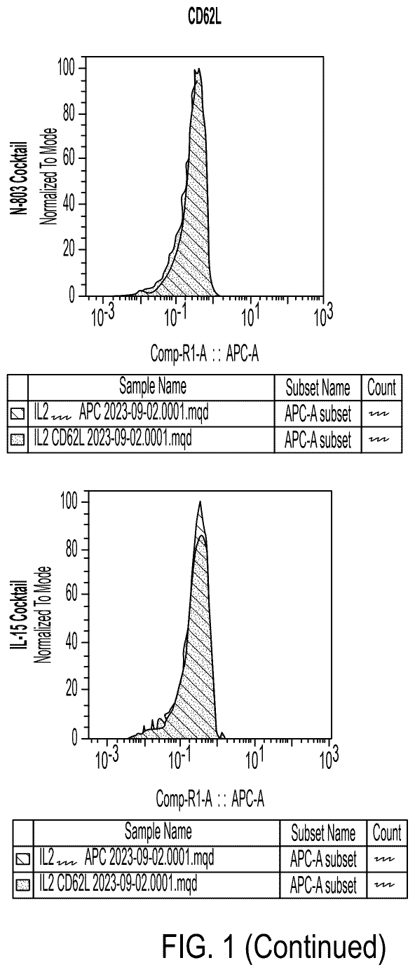

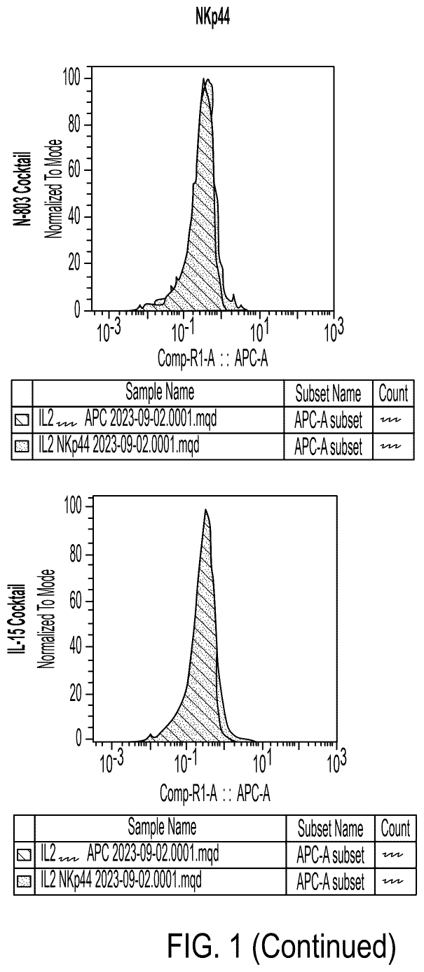

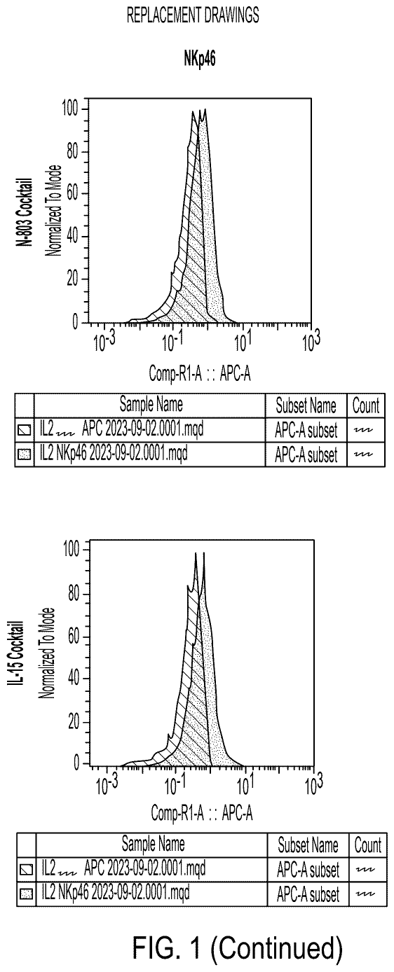

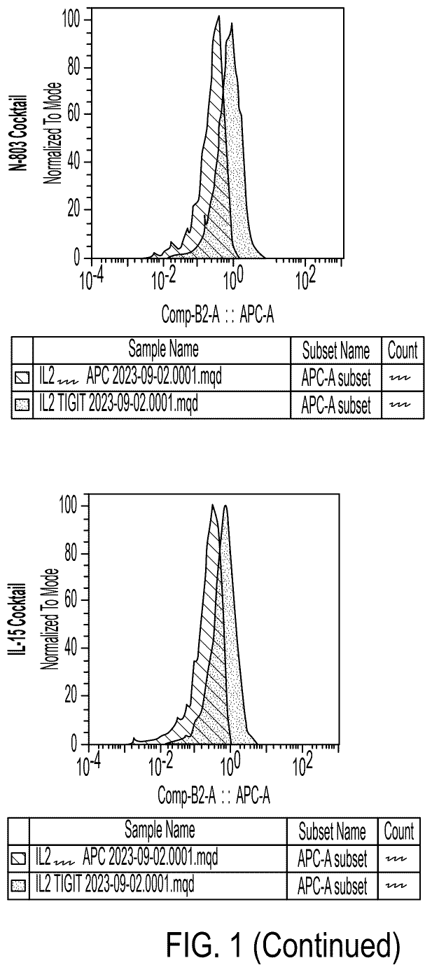

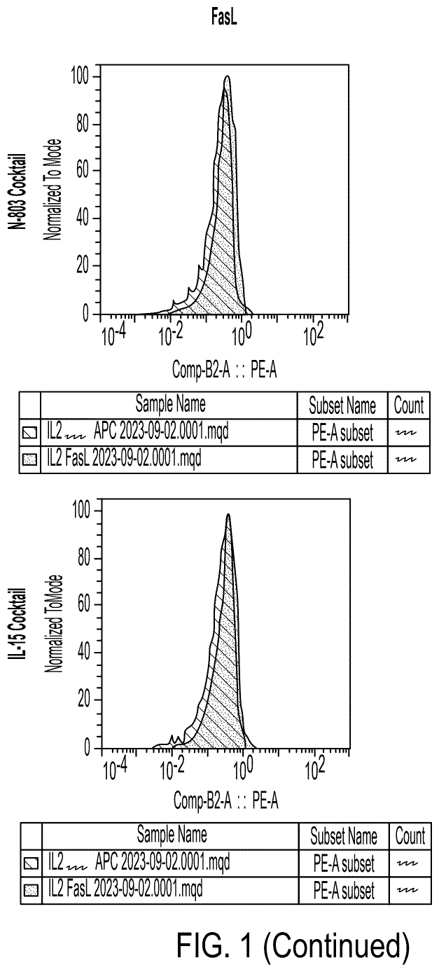

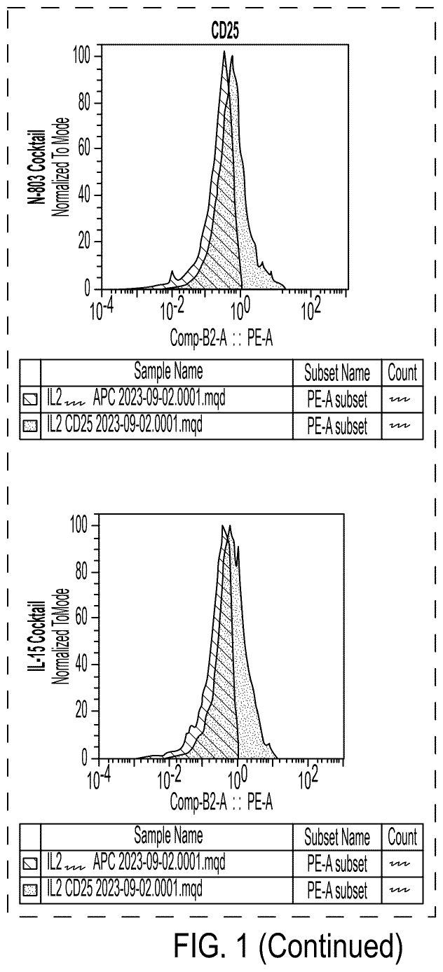

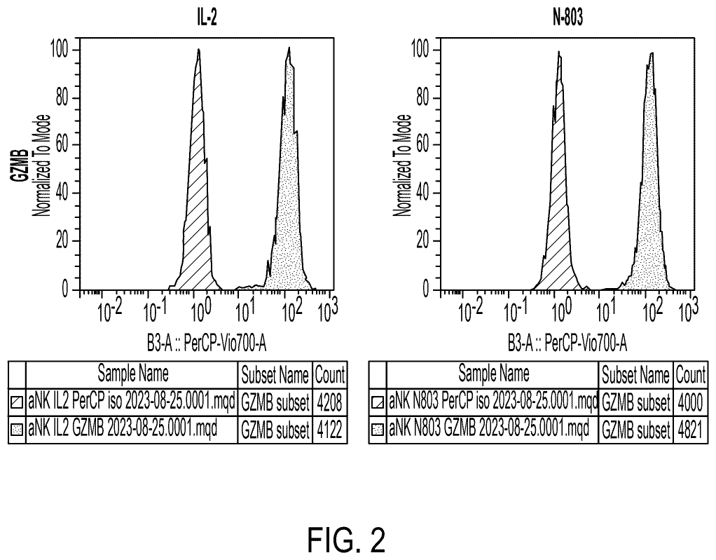

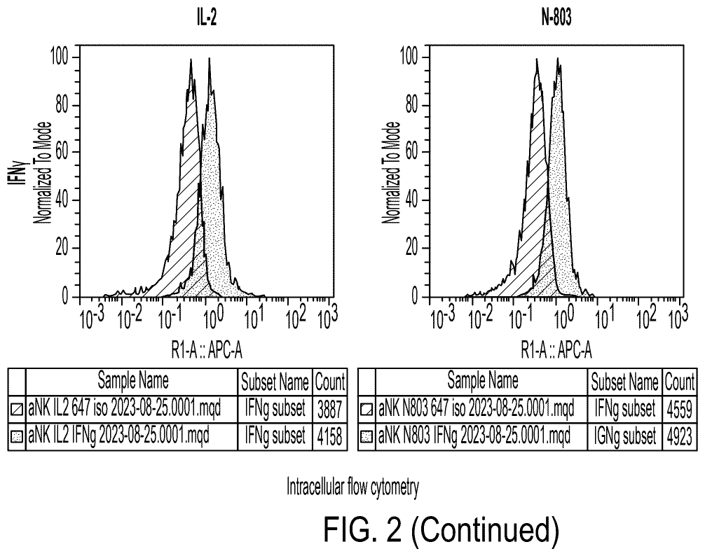

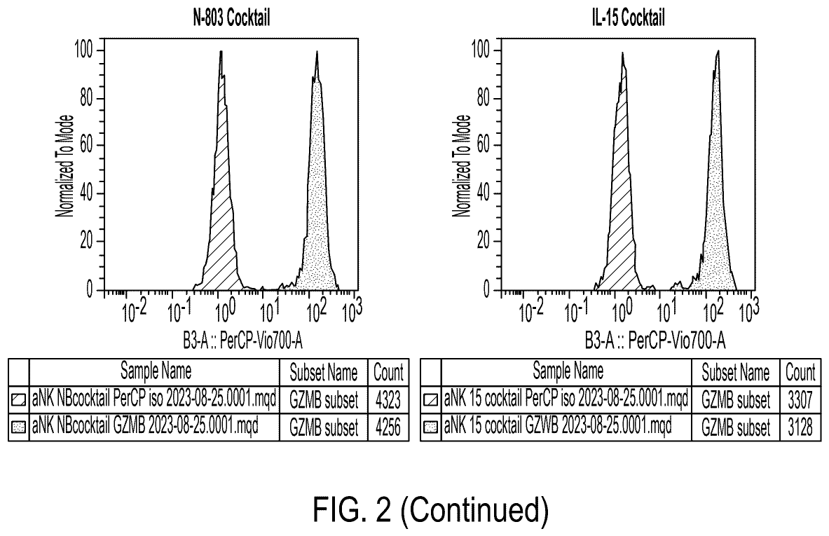

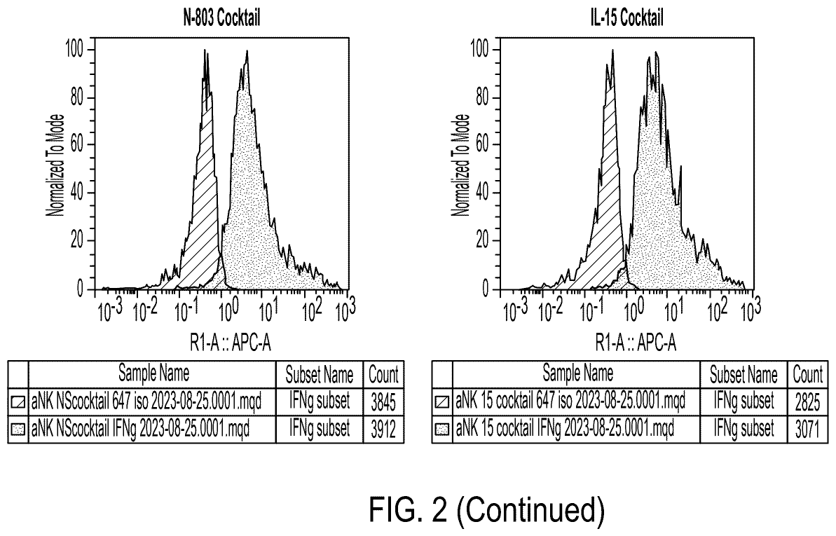

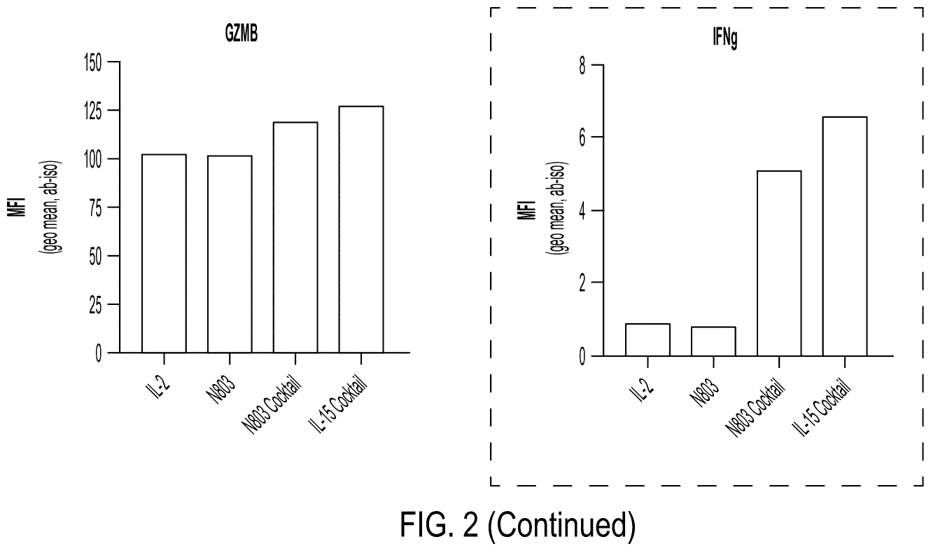

Regular cell surface and intracellular flow analysis performed the next day shows that the combination of IL-12, IL-18, and IL-15 or N-803 triggered CIML-like characteristics such as increased CD25, IFNγ, and GranzymeB expression. Exemplary results are depicted in FIG. 1 and FIG. 2.

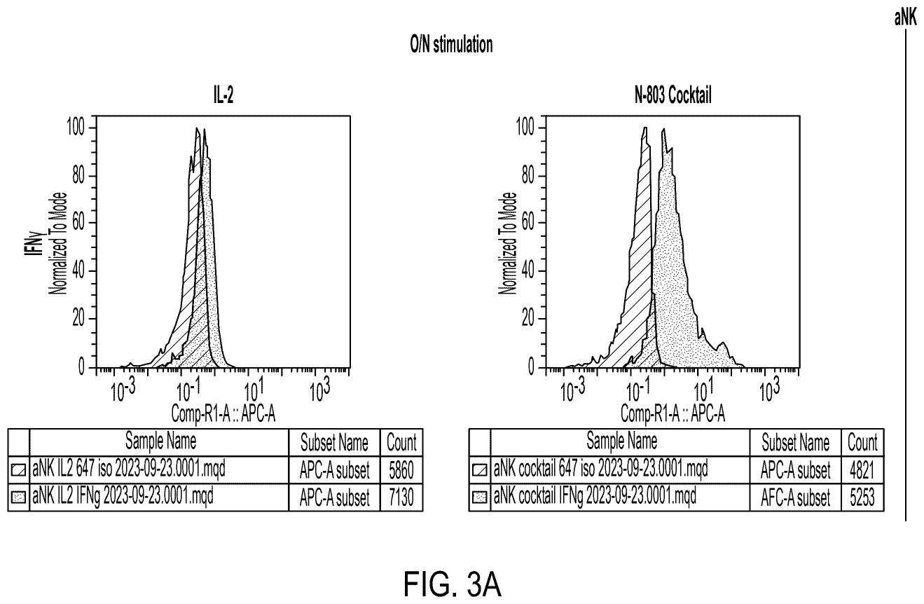

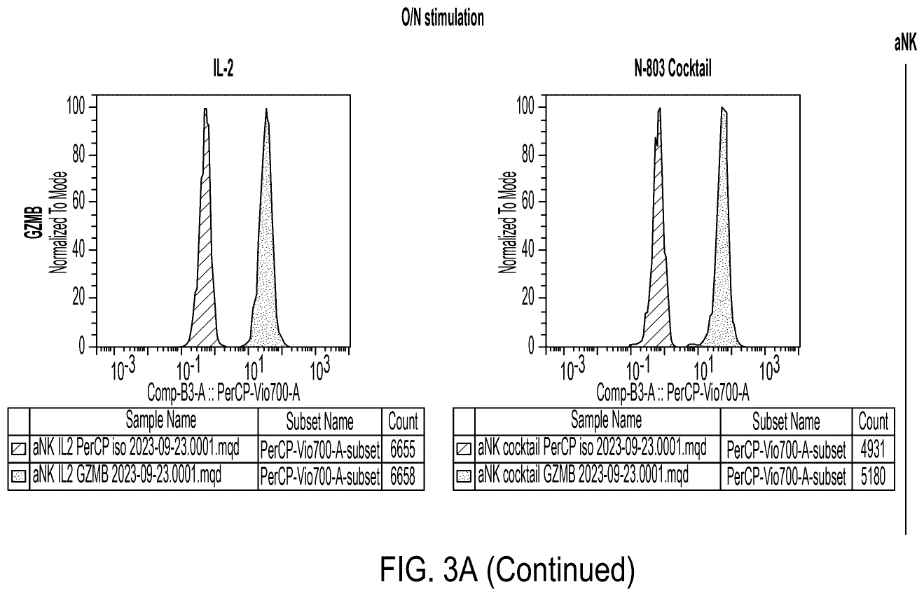

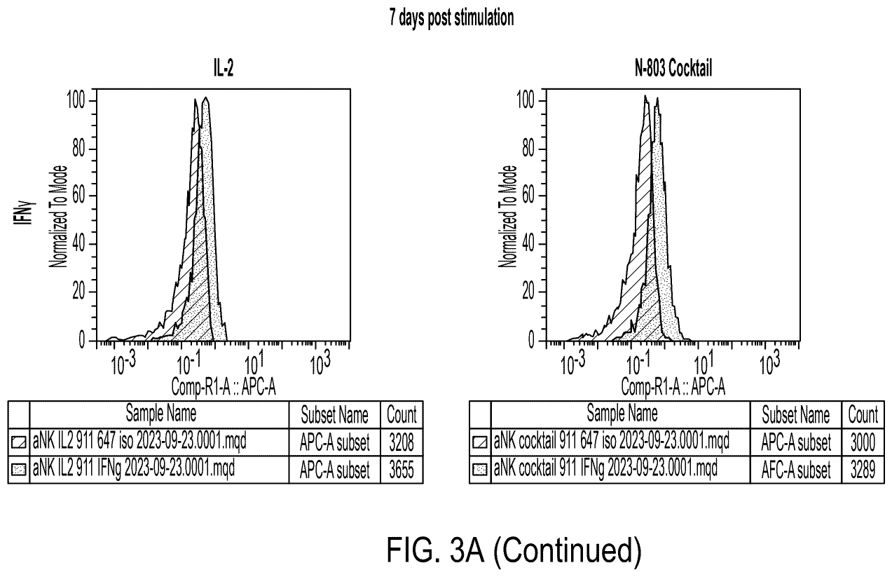

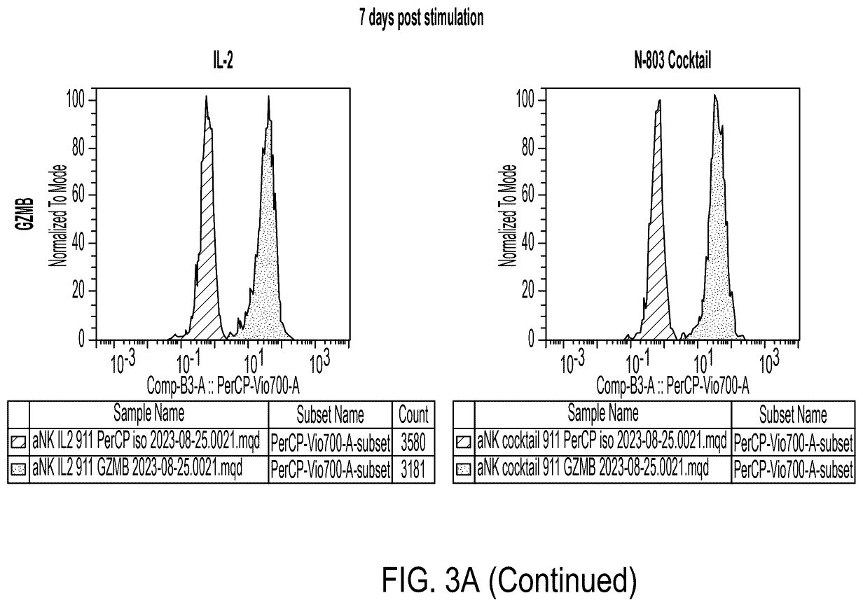

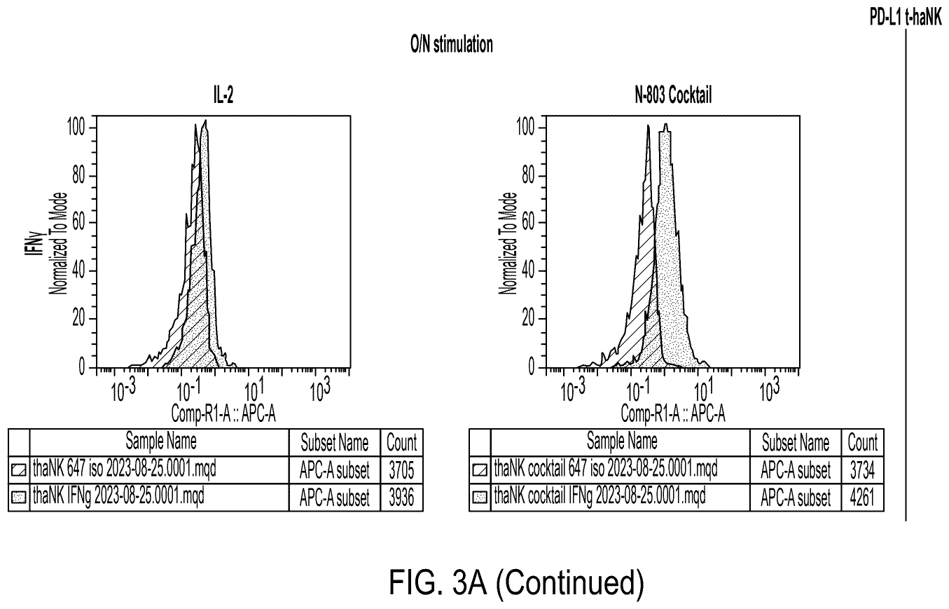

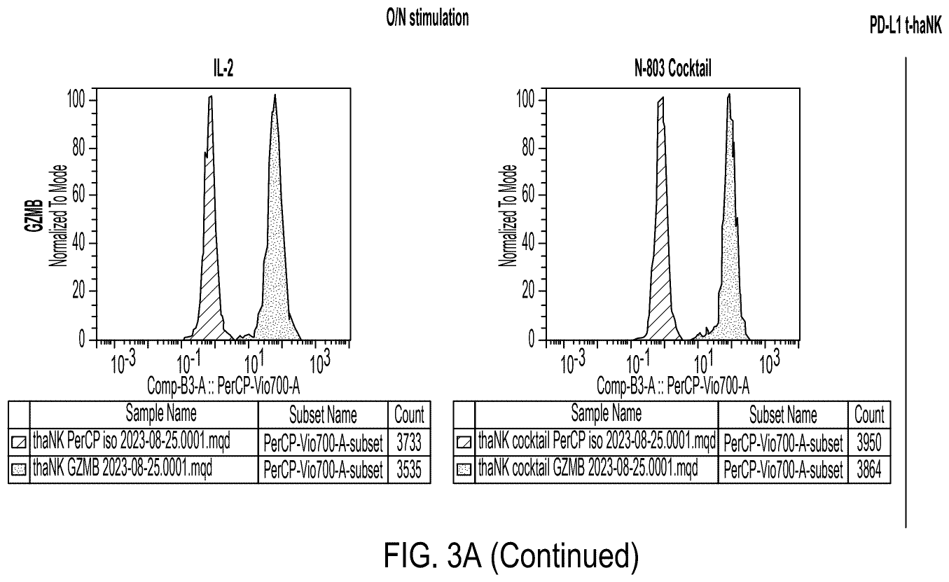

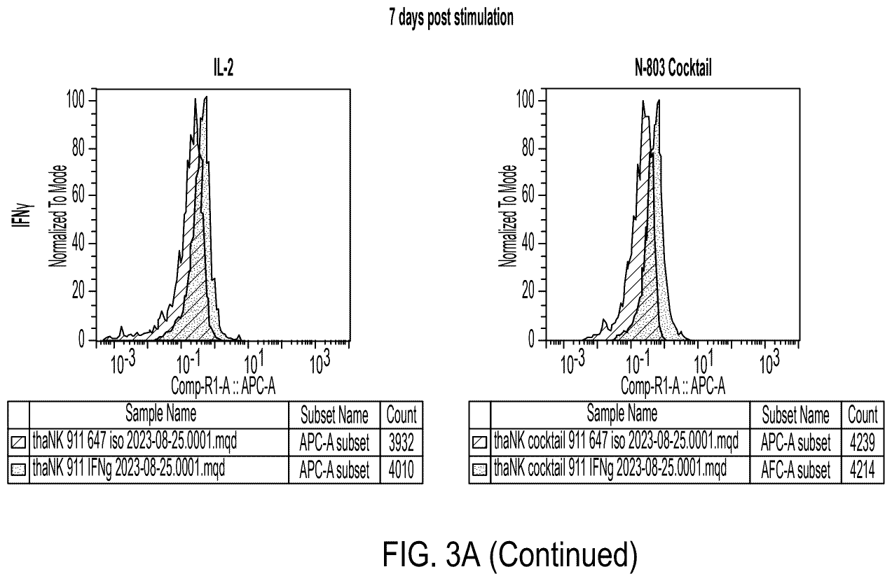

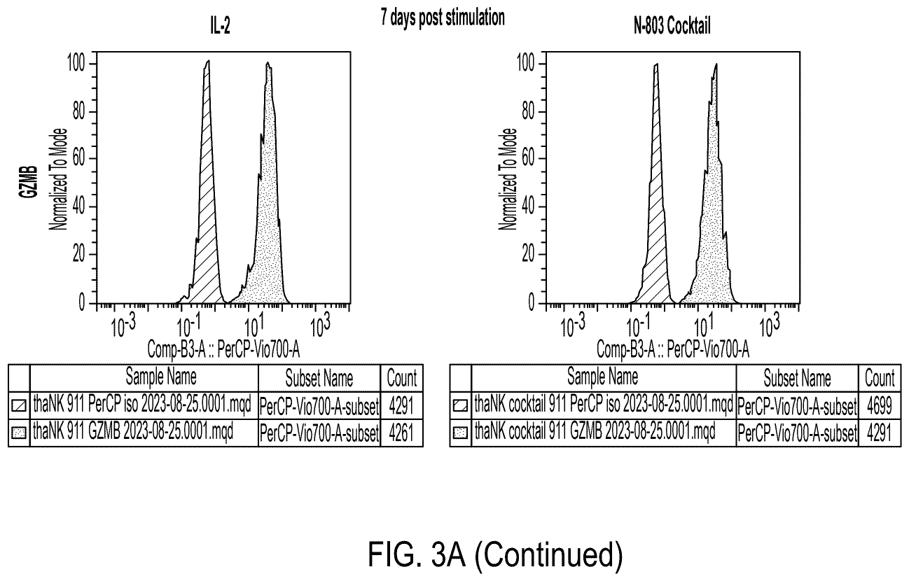

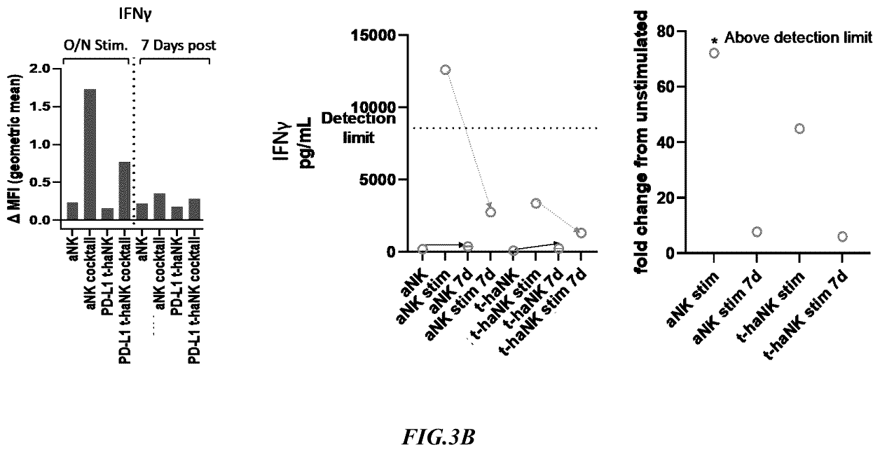

Cytokines-stimulated aNK and PD-L1 t-haNK cells were washed, put back in regular growth medium for several days (with IL-2 supplement at 500 IU/mL for aNK). The cells were either analyzed by intracellular flow cytometry or their culture supernatant analyzed by ELISA. 7 days after the cytokines-stimulation step, aNK and PD-L1 t-haNK both show sustained increase in IFNγ expression as can be seen from FIGS. 3A and 3B. CIML-stimulation induced up to 7.5-fold increase in intracellular IFNγ level in aNK cells. Induction of intracellular IFNγ was durable, with a 1.5-fold increase over steady-state aNK culture 7 days after CIML-stimulation. CIML-stimulation was also effective on PD-L1 t-haNK cells. FIG. 3B further showed that CIML-stimulation induced >45-fold increase in secreted IFNγ in aNK and PD-L1 t-haNK cells. Induction of secreted IFNγ was durable, with a 6-8-fold increase_over steady-state aNK culture 7 days after CIML-stimulation.

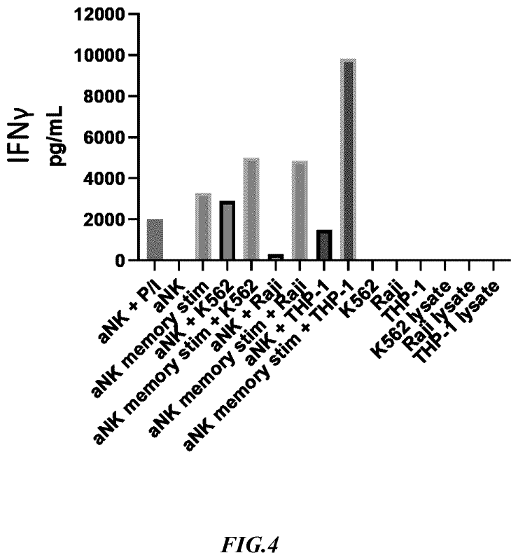

Cytokines-stimulated aNK and PD-L1 t-haNK cells were washed, put back in regular growth medium for several days (with IL-2 supplement at 500 IU/mL for aNK). Cells were co-incubated with target cells after 7 and 14 days and the co-incubation supernatants were analyzed by ELISA. Cytokines-treated aNK and PD-L1 t-haNK cells display synergistically increased secretion of IFNγ upon stimulation by target cells, compared to non-stimulated cells or non-treated cells co-incubated with targets. Exemplary results are depicted in FIG. 4. Remarkably, synergistic behavior can be observed for up to 14 days in culture, depending on the target cells used as stimulant. Such a memory recall behavior is a hallmark of CIML-NK cells presented herein as is also shown in FIG. 5. These figures show that cytokine cocktail-treated aNK cells strongly respond to stimulation by target cells; strength of IFNγ response is dependent on the target used. Strong IFNγ response to stimulation by target cells observed 1 week after initial cytokine cocktail treatment=“memory recall” phenotype. IFNγ “memory recall” response still detectable 2 weeks after initial cytokine cocktail treatment (Raji targets). Sustained increase in IFNγ expression, lasting up to 7 days after initial stimulation. Memory Recall: Increased IFNγ response to stimulation by target cell engagement lasts at least 7 days post CIML-stimulation.

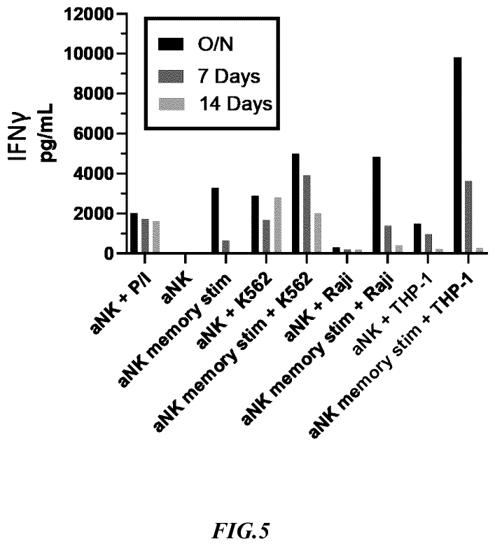

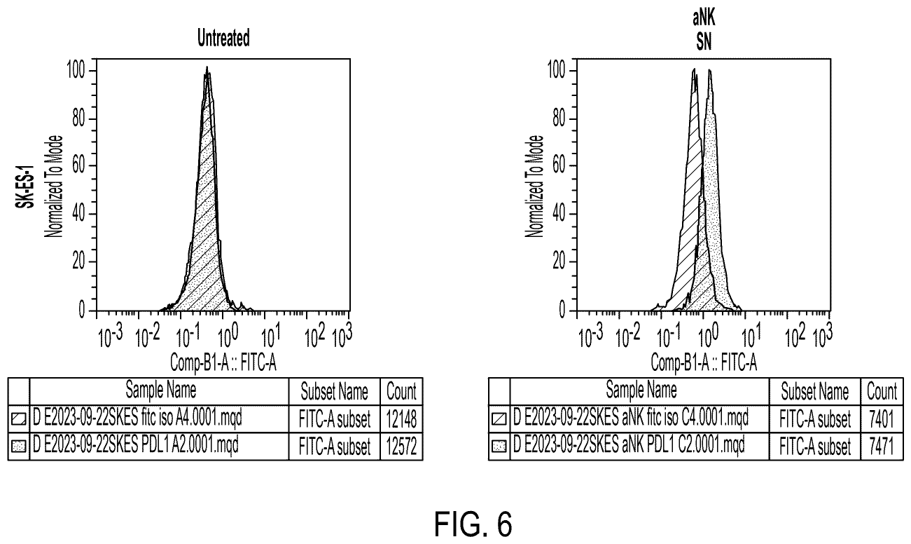

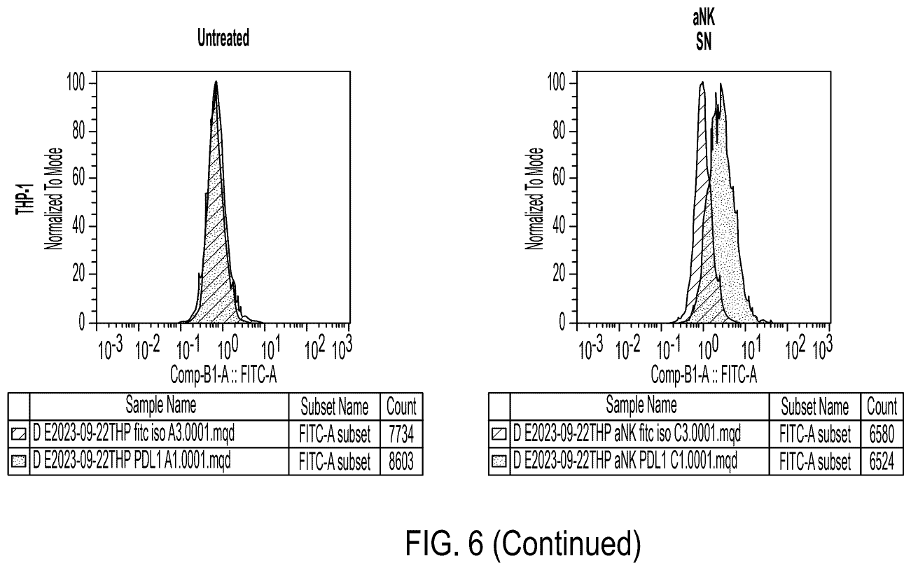

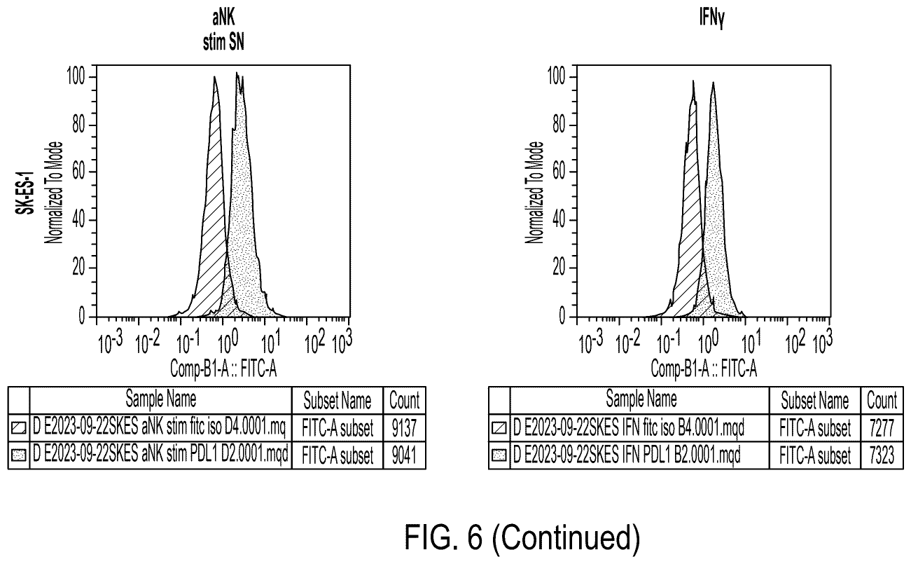

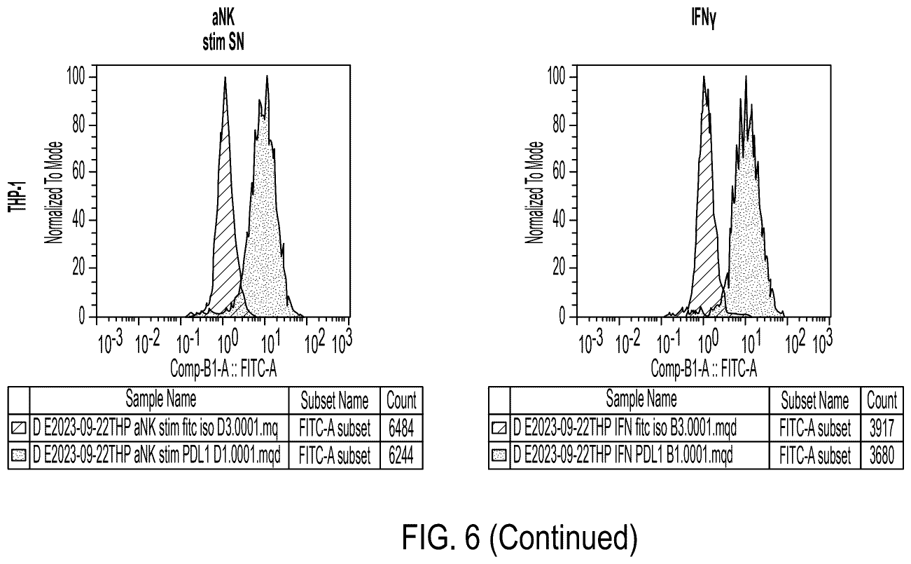

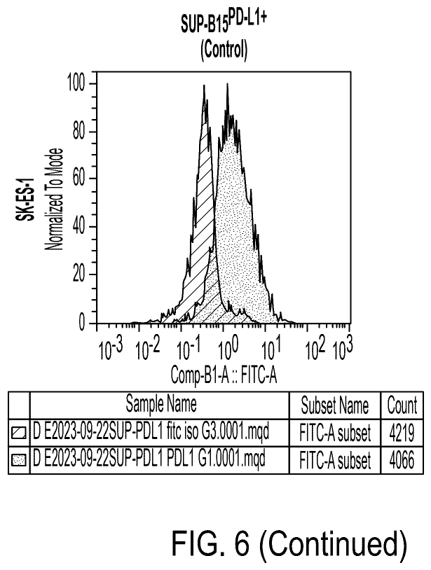

Cytokines-stimulated aNK cells were washed and put back in regular growth medium with IL-2 supplement at 500 IU/mL. The next day, culture supernatants of stimulated or unstimulated aNK cultures were collected and applied to target cells for 48 hrs. Flow analysis of the treated target cells shows increased upregulation of surface PD-L1 in cells treated with CIML-like aNK supernatant compared to regular aNK culture supernatant. Exemplary results are depicted in FIG. 6.

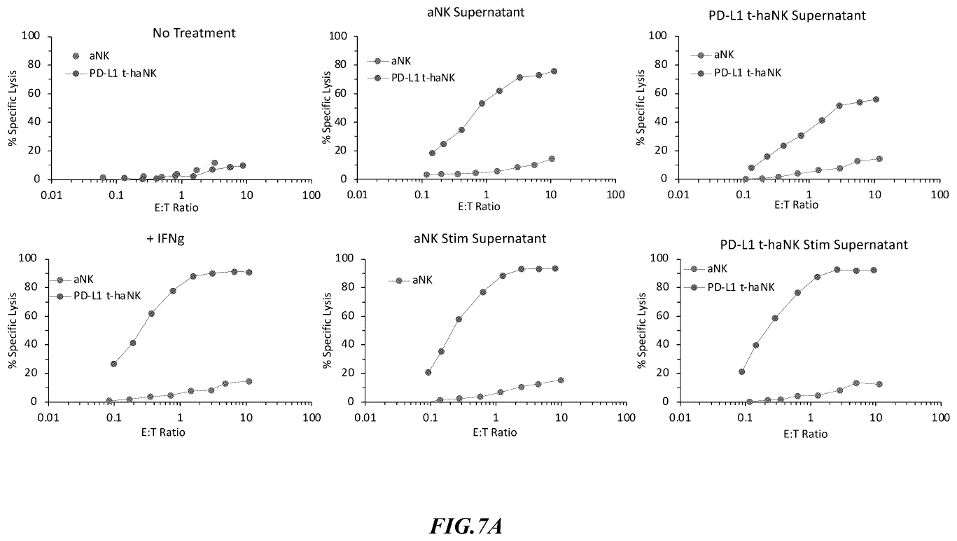

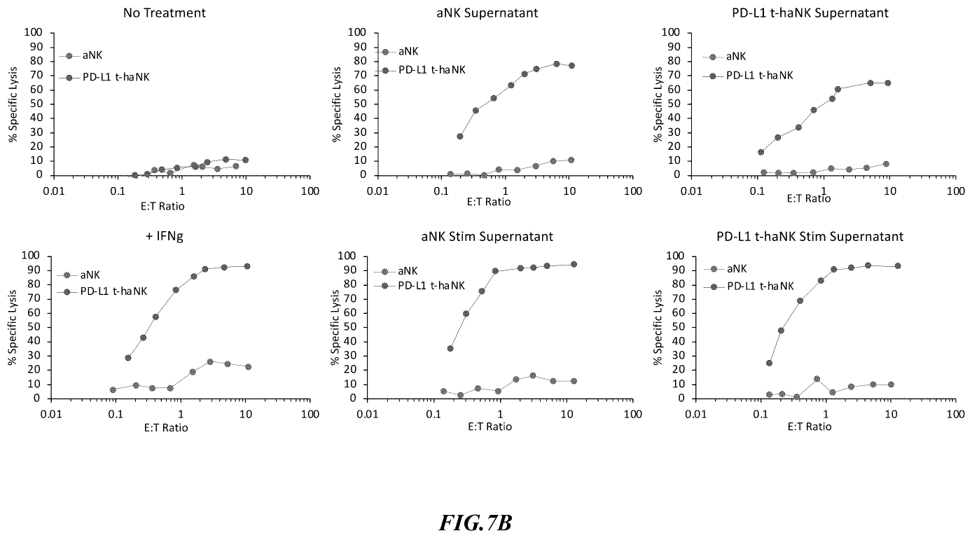

Cytokines-stimulated aNK and PD-L1 t-haNK cells were washed, put back in regular growth medium for several days (with IL-2 supplement at 500 IU/mL for aNK). The next day, culture supernatants of stimulated or unstimulated cultures were collected and applied to aNK-resistant target cells for 48 hrs. In vitro cytotoxicity assays were then performed on the supernatant-treated target cells using aNK or PD-L1 t-haNK cells as effectors. Target cells treated with regular aNK or PD-L1 t-haNK supernatant display some increased sensitivity to killing by PD-L1 t-haNK, while remaining resistant to killing by aNK. This sensitivity to PD-L1 t-haNK was markedly increased in target cells that were treated with CIML-like cell supernatants as can be readily seen from FIG. 7A (where CIML-aNK supernatant increases SK-ES-1 sensitivity to PD-L1-t-haNK) and FIG. 7B (where CIML-aNK supernatant increases THP-1 sensitivity to PD-L1-t-haNK). Supernatant from CIML-treated aNK and PD-L1 t-haNK cells makes SK-ES-1 and THP-1 highly and selectively sensitive to killing by PD-L1 t-haNK effector cells. The above data and experiments show that aNK cells acquire CIML-like properties upon O/N stimulation with IL-12/N-803/IL-18 cytokines cocktail, thereby forming memory-like aNK cells (M-aNK cells). M-aNK culture supernatant “super-induces” PD-L1 expression on NK-resistant target cells and increases susceptibility of resistant target cells to killing by PD-L1 t-haNK cells. The inventors contemplate that the effect is most likely mediated by IFNγ.

In still further aspects of the inventive subject matter, the inventors contemplate that RNAseq analysis of the cells contemplated herein is performed before memory induction with cytokines cocktail, right after induction, post (e.g., 3-4 days) induction, and at time of re-challenge with a target cell or cytokine. Thusly obtained results are then used to further characterize or modulate (e.g., amplify) desired features, for example, by recombinant overexpression of upregulated genes or silencing down-regulated genes. Moreover, RNAseq data is used to inform which cytokine/chemokine/surface molecules to verify by flow or ELISA assays.

In still further experiments, optimal irradiation levels can be identified to ascertain which irradiation levels affect the IFNγ “memory-recall” behavior after re-challenge of mem-like aNK cells with target cells. Similarly, it is contemplated that the PD-L1 “feedback” loop can be further examined in vivo in NSG mice implanted with human tumors (like SK-ES-1 or THP-1). Here, PD-L1 t-haNK cells are infused and their activity compared with corresponding non-induced cells. Where the IFNγ-induced PD-L1 feedback is in effect, memory-like PD-L1 t-haNK cells are better at controlling tumors than regular PD-L1 (or even memory-like aNK). This model can also be used to test the immediate memory-like phenotype, as well as the “re-challenge” model where memory-like PD-L1 t-haNK cells are rested for a few days following the cytokines cocktail induction step.

Additionally, and as already noted above, it is contemplated that the CIML-aNK cells and derivatives thereof can be subjected to further engineering that adds a further safety feature such as a suicide gene, and/or that add one or more secreted or intracellularly retained cytokine (e.g., IL-15), a trap for immune suppressive molecules (e.g., TGFβ), etc. As will be readily appreciated, any recombinant gene expressed in the cells contemplated herein can be expressed constitutively or in an inducible fashion.

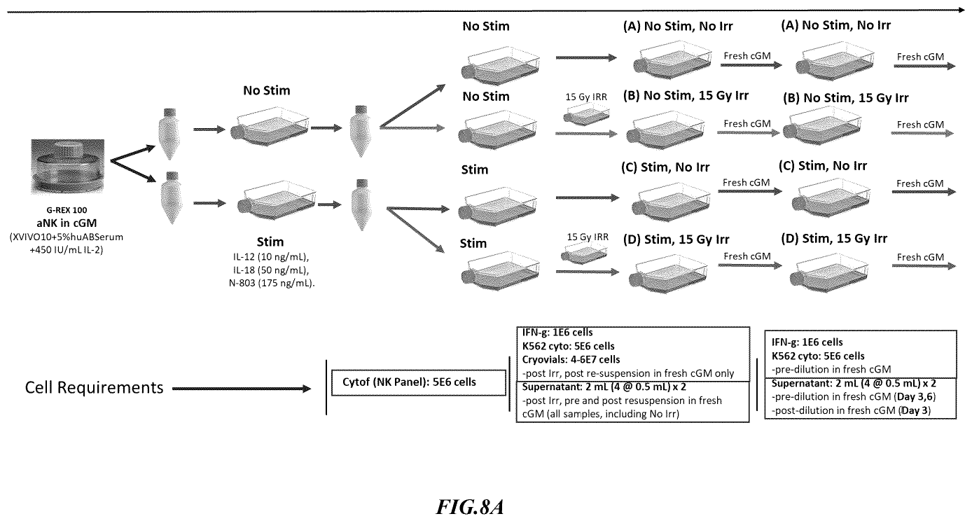

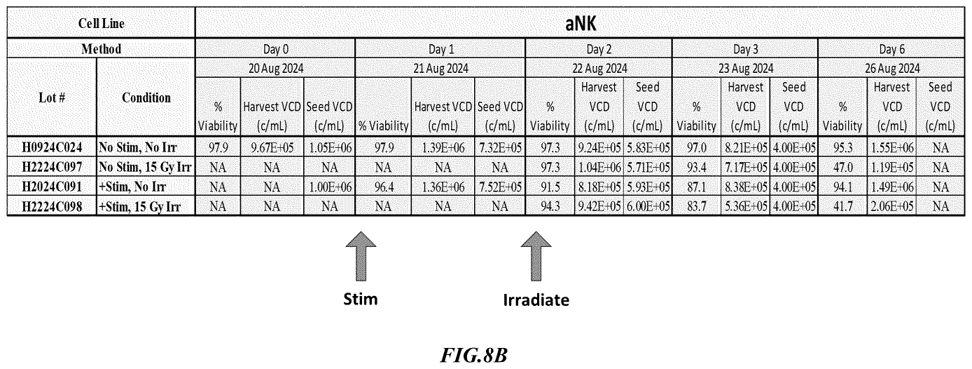

FIG. 8A provides a flowchart and FIG. 8B provides a table comprising laboratory execution of the CIML induction and sampling protocol of aNK cells. On Day 0, 1.5×10⁸ cells were transferred into centrifuge tubes, centrifuged at 336×g for 15 min, resuspended pellet at 1E6 cells/mL in 150 mL No Stim/Stim media. Transferred cells into T-225 flasks. On Day 1, cell count was performed, transferred all cells into centrifuge tubes, centrifuged at 336×g for 15 min, resuspended pellet at ˜7E5 cells/mL in fresh cGM. Divided volume equally into two T-225 flasks per condition. Allowed overnight recovery in fresh cGM. On Day 2, cell counts were taken. A subset of the cells (B and D) was irradiated at 15 Gy in T-Flask. Take pre-resus samples. Centrifuged cells (all conditions) at 336×g for 15 min, resuspended pellet at ˜7E5 cells/mL in fresh cGM. Performed cell counts and made aliquots for fresh sample testing. Cryopreserved 2-3 vials per condition (˜2E7 c/mL in 1:1 (HA:CS10); Allowed overnight incubation in fresh cGM. On day 3, cell counts were performed and made aliquots for fresh sample testing. Maintained cells appropriately for culture over the weekend. On Day 6, same sample collection was done as Day 3.

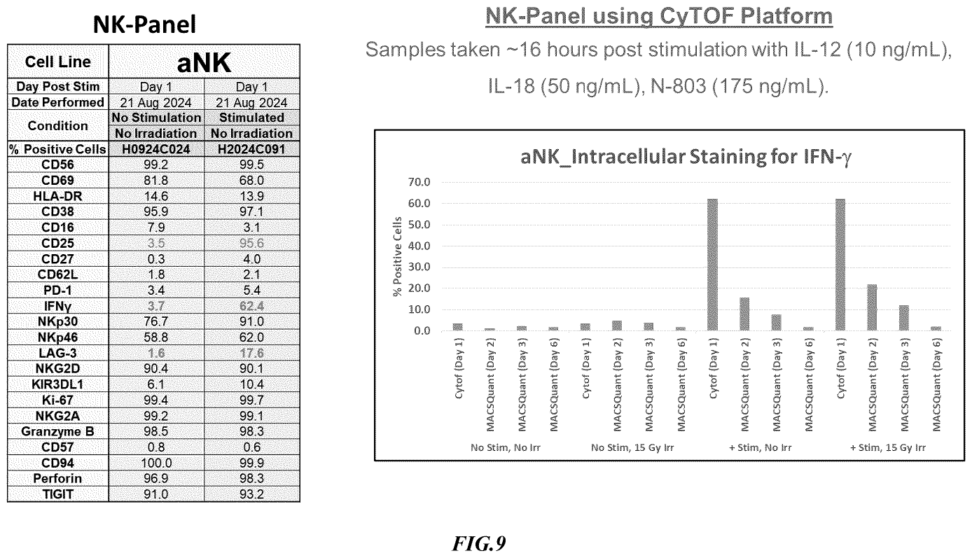

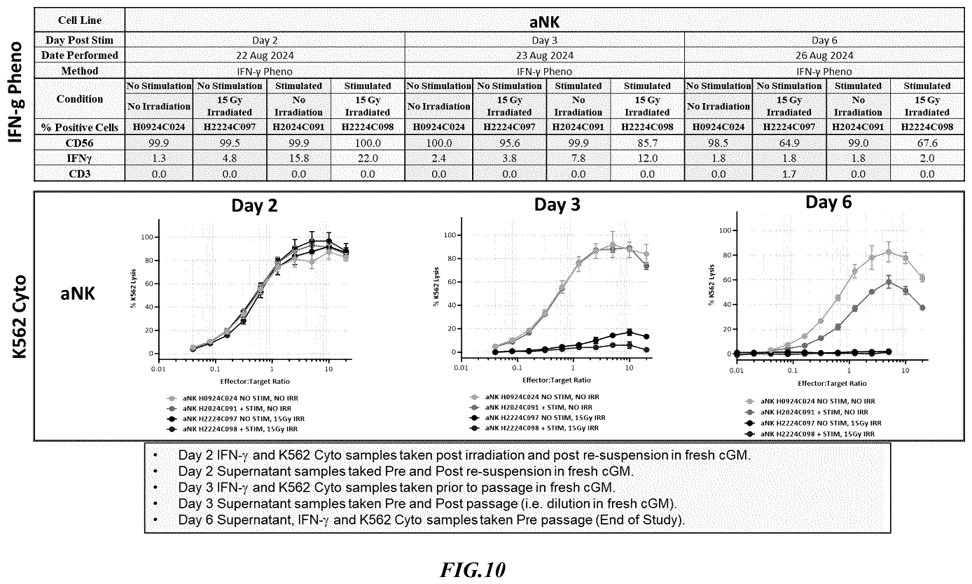

FIGS. 9-10 and Tables 2-3 provide an experimental workflow and outlines detailed timelines for cytokine stimulation, irradiation, and sampling. IL-12 (10 ng/mL), IL-18 (50 ng/mL), and N-803 (175 ng/mL) used for ˜16 hours. Cells then washed and cultured in fresh complete growth medium (cGM) with IL-2 supplementation. Key sampling points: Day 0 (stim), Day 1, Day 2, Day 3, Day 6. Irradiation (15 Gy) performed on certain groups to mimic clinical preparation.

TABLE 2
Cryopreserved Cell Sample List

| | | | | | | # of | |
|---|---|---|---|---|---|---|---|
| | Sample | | Timing of | | | Vials at | |
| | Label/ | Sample | Sample | | Date of | (2 × 10⁷ | |
| Sample Type | Conditions | Lot# | Harvest | Timepoint | Sample | cells/vial) | Location |
| Cryopreserved | aNK (NO STIM, | H0924C024 | Post- | Day 2 | 22 Aug. | 3 | Cryo B, Rack 7, |
| Cells | NO IRR) | | Resuspension | | 2024 | | Box 7-1 |
| | | | in fresh cGM | | | | |
| Cryopreserved | aNK (NO STIM, | H2224C097 | Post- | Day 2 | 22 Aug. | 3 | Cryo B, Rack 7, |
| Cells | 15Gy IRR) | | Resuspension | | 2024 | | Box 7-1 |
| | | | in fresh cGM | | | | |
| Cryopreserved | aNK (+STIM, | H2024C091 | Post- | Day 2 | 22 Aug. | 3 | Cryo B, Rack 7, |
| Cells | NO IRR) | | Resuspension | | 2024 | | Box 7-1 |
| | | | in fresh cGM | | | | |
| Cryopreserved | aNK (+STIM, | H2224C098 | Post- | Day 2 | 22 Aug. | 3 | Cryo B, Rack 7, |
| Cells | 15Gy IRR) | | Resuspension | | 2024 | | Box 7-1 |
| | | | in fresh cGM | | | | |

TABLE 3
Cryopreserved Vial thaw

| | Condition | | | Phenotype % Positive | | | | | |
|---|---|---|---|---|---|---|---|---|---|
| Study | ID | Lot # | Condition | CD3 | CD56 | CD16 | CD54 | NKG2D | NKp30 |
| TP552.04 | A | H0924C024 | aNK Fresh | 0.96 | 99.82 | 5.46 | 99.58 | 97.84 | 96.42 |
| | | | Reference | | | | | | |
| TP552.04 | B | H0924C024 | aNK − Stim − | 0.46 | 99.47 | 3.67 | 99.14 | 93.49 | 86.61 |
| | | | IRR | | | | | | |
| TP552.04 | C | H2224C097 | aNK − Stim + | 0.74 | 99.36 | 3.53 | 99.02 | 93.25 | 89.55 |
| | | | IRR | | | | | | |
| TP552.04 | D | H2024C091 | aNK + Stim − | 0.87 | 99.01 | 4.93 | 99.36 | 85.51 | 87.03 |
| | | | IRR | | | | | | |
| TP552.04 | E | H2224C099 | aNK + Stim + | 0.97 | 98.99 | 3.71 | 99.26 | 84.68 | 87.56 |
| | | | IRR | | | | | | |

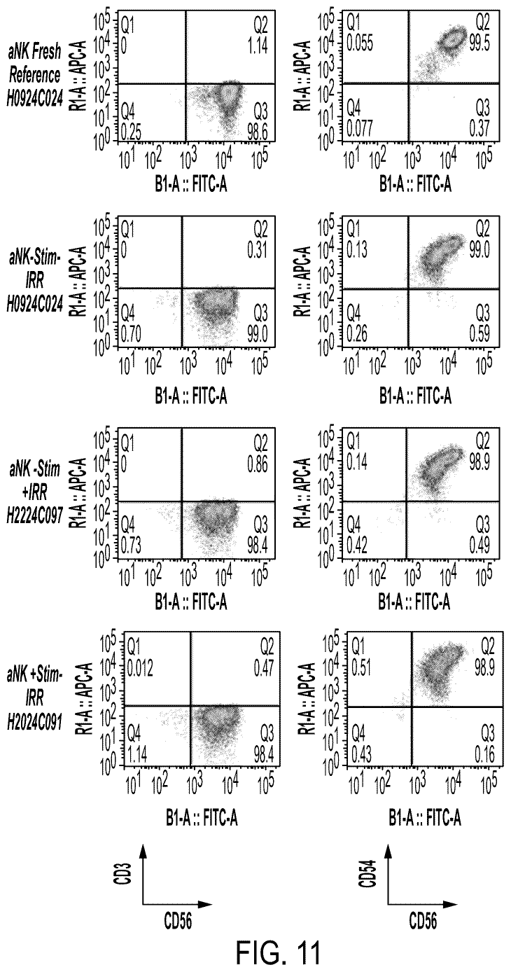

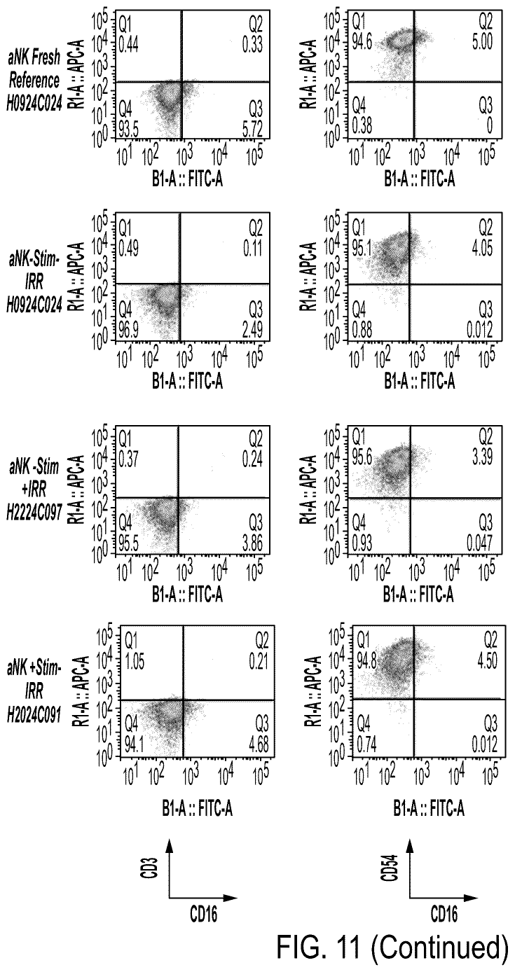

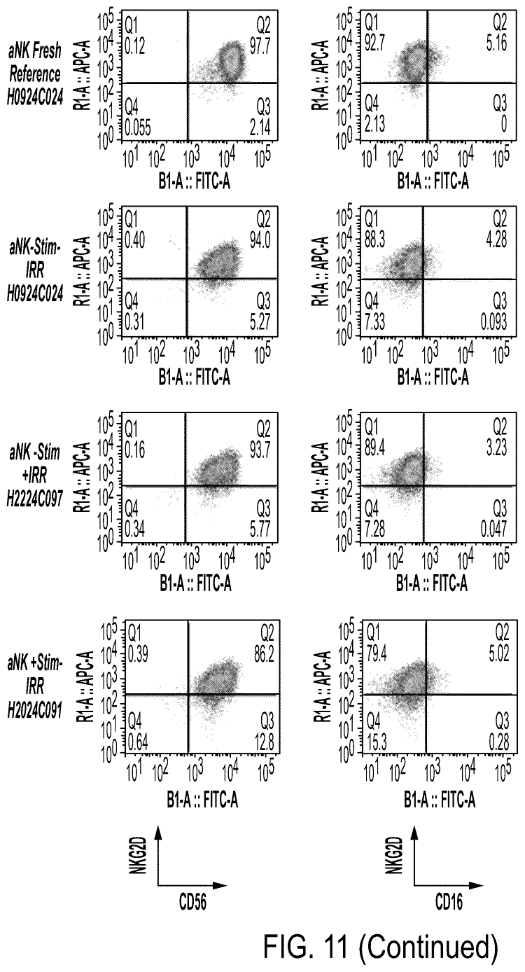

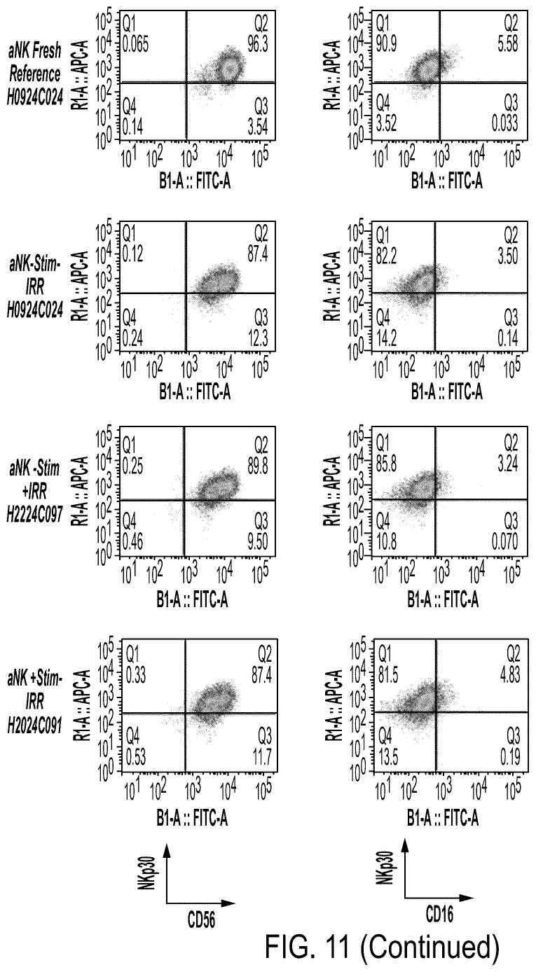

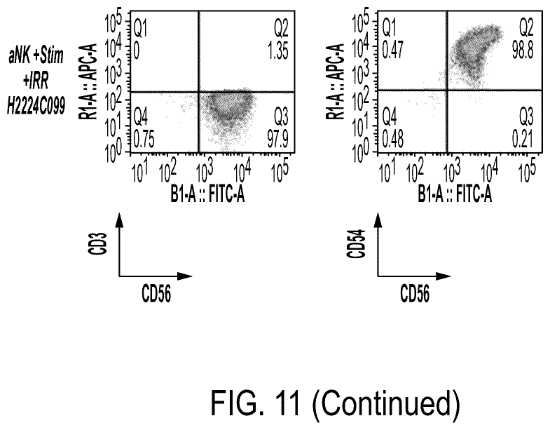

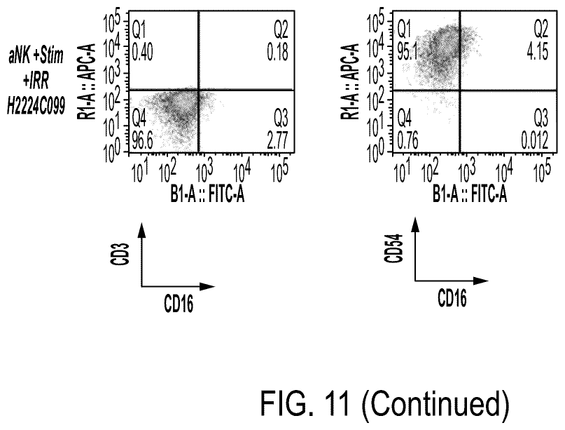

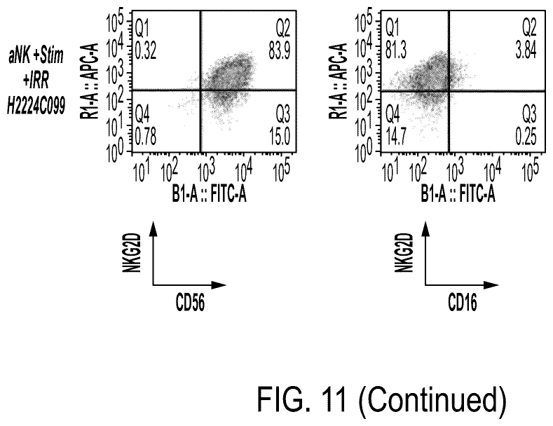

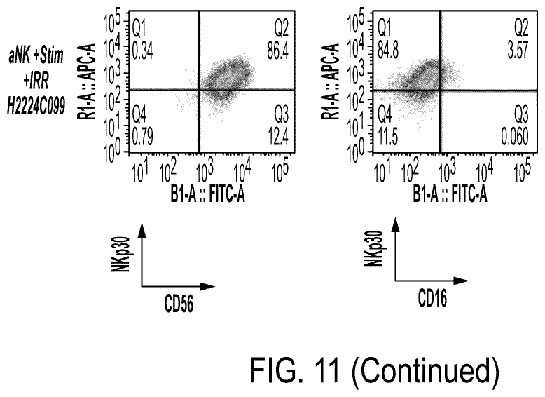

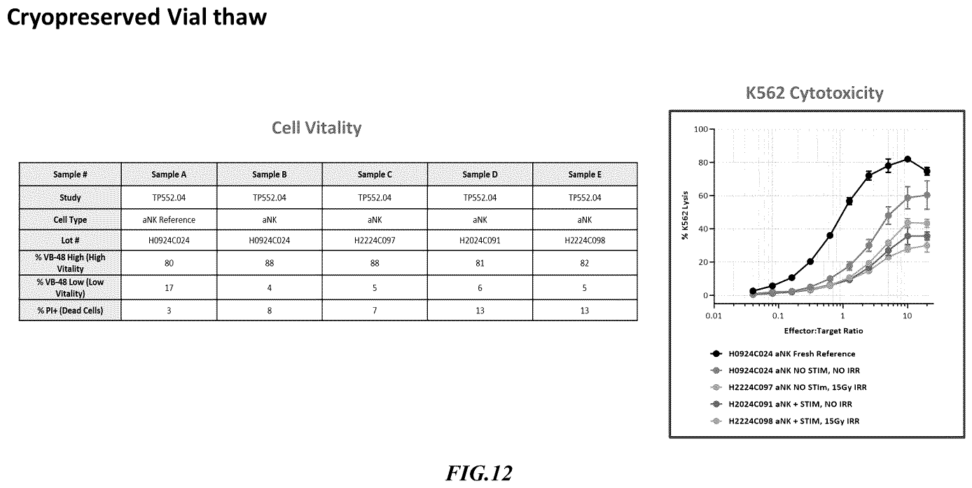

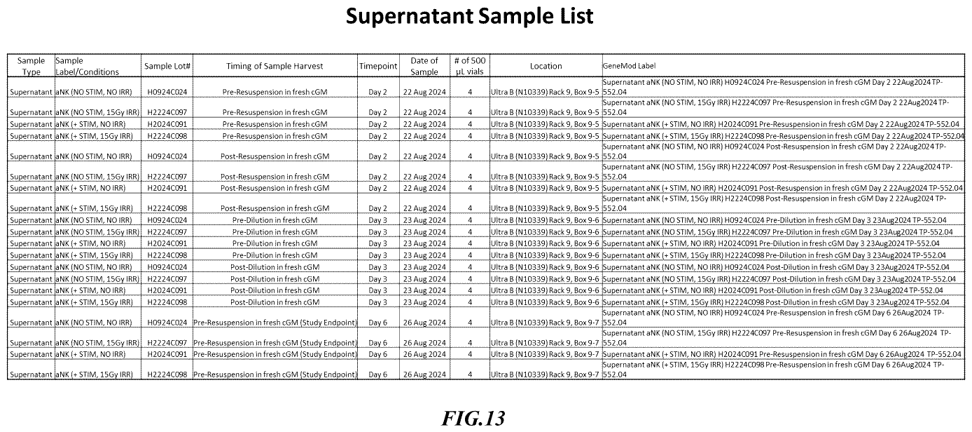

FIGS. 11-13 further define IFNγ samples (1×10⁶ cells), Cytotoxicity assays (K562, 5×10⁶ cells), Supernatant volumes and timing (before/after cGM exchange), Cryopreserved aliquots (2-3 vials per condition, ˜2×10⁷ cells/mL).

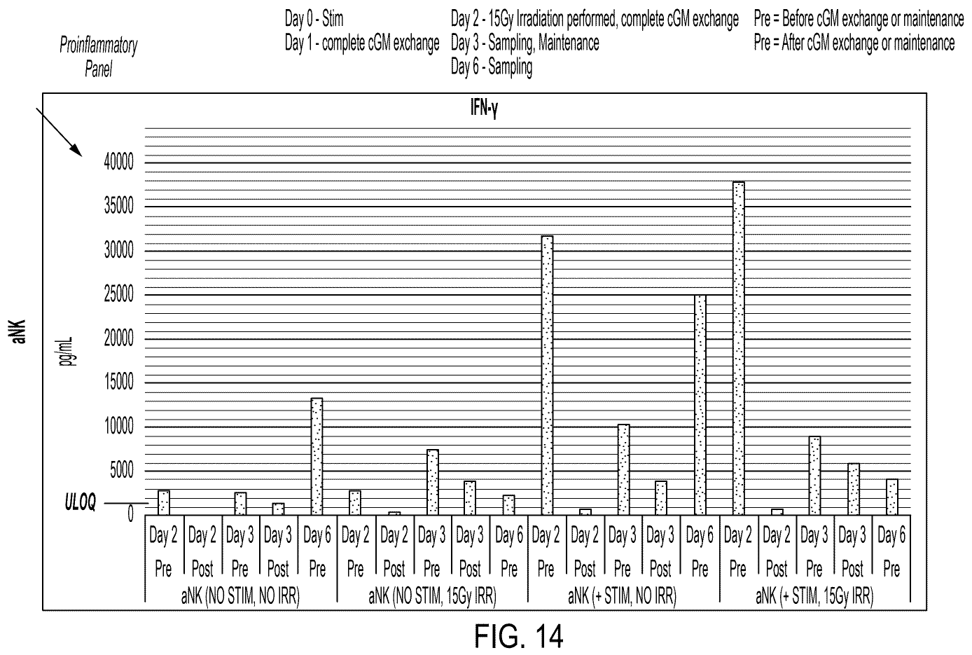

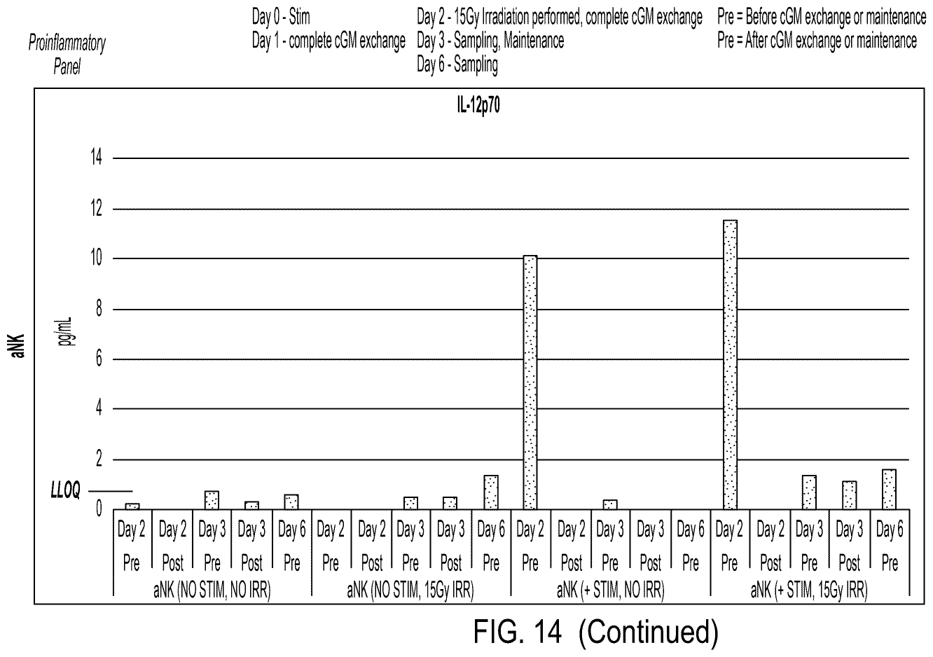

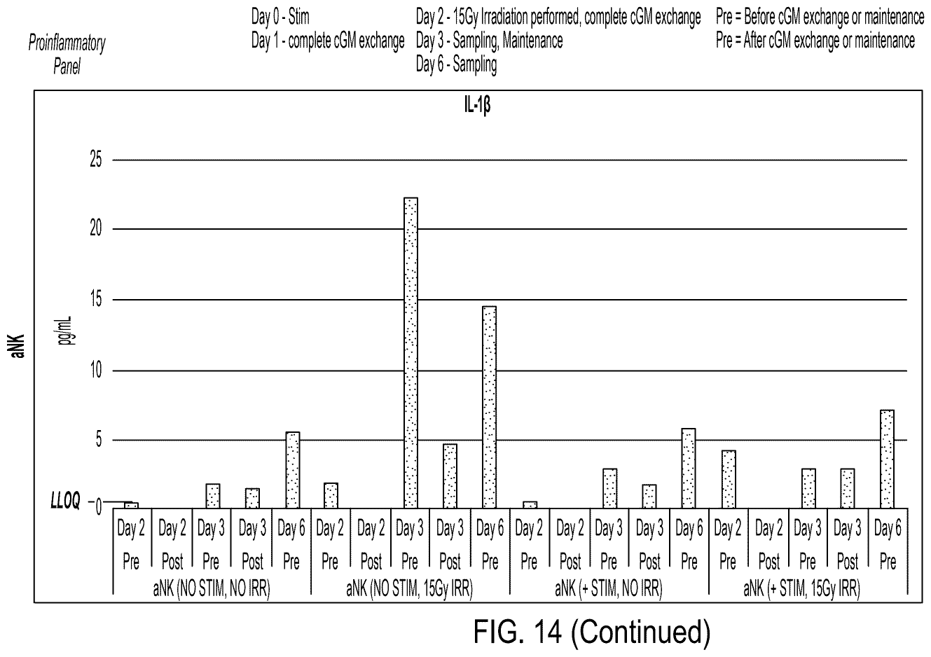

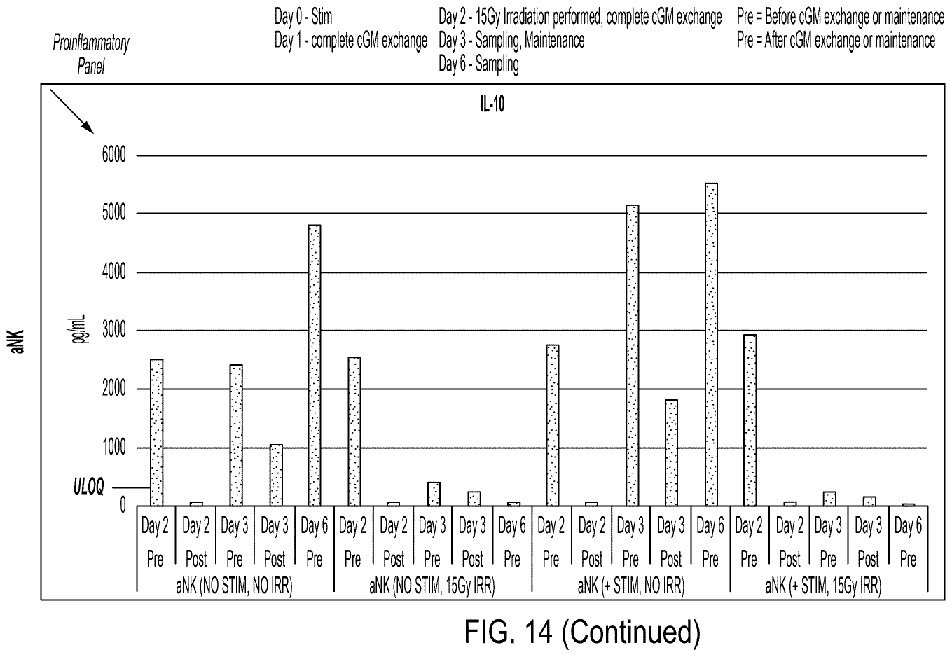

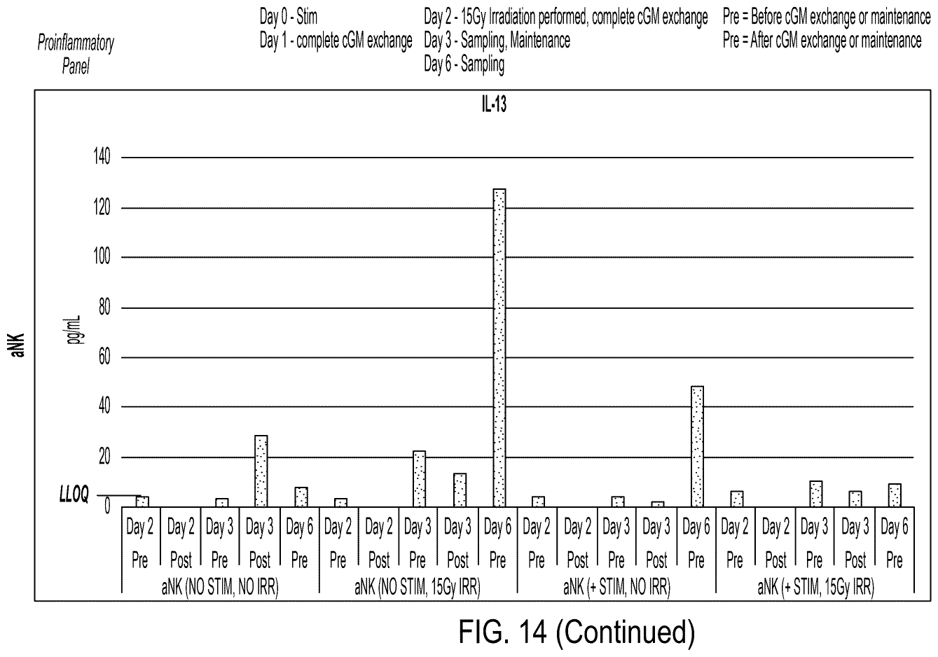

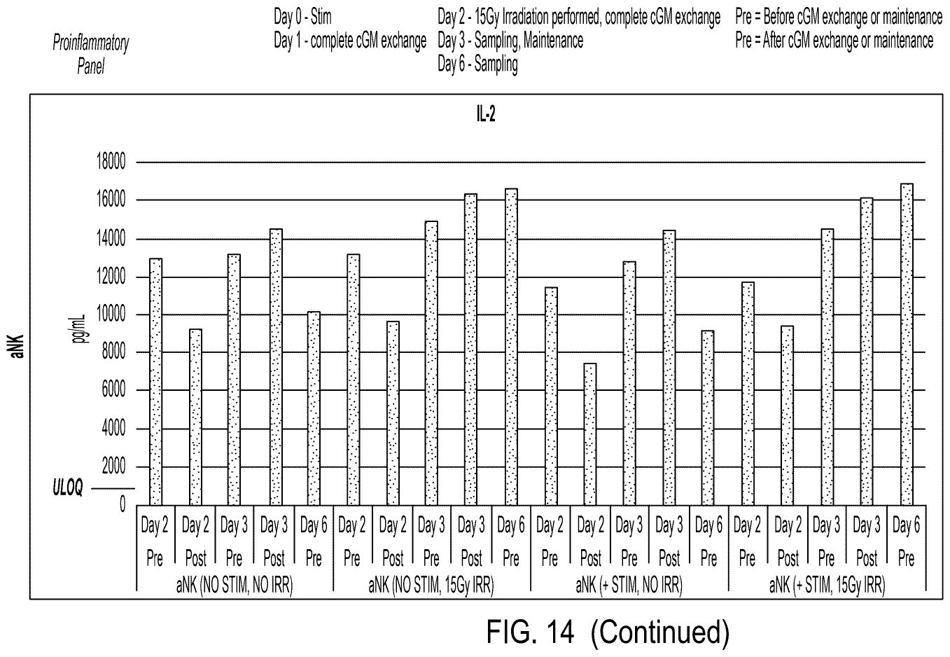

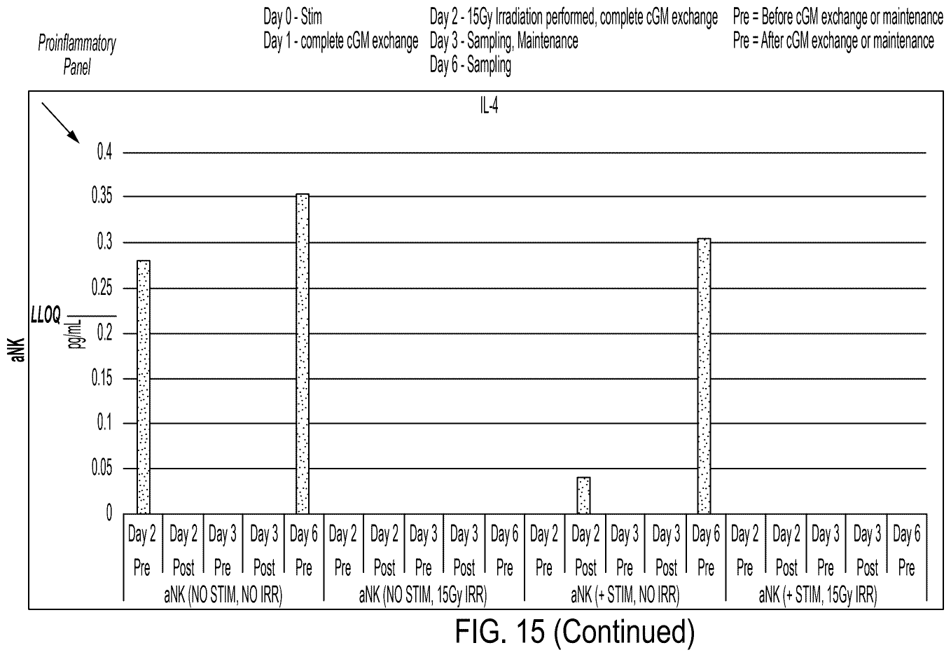

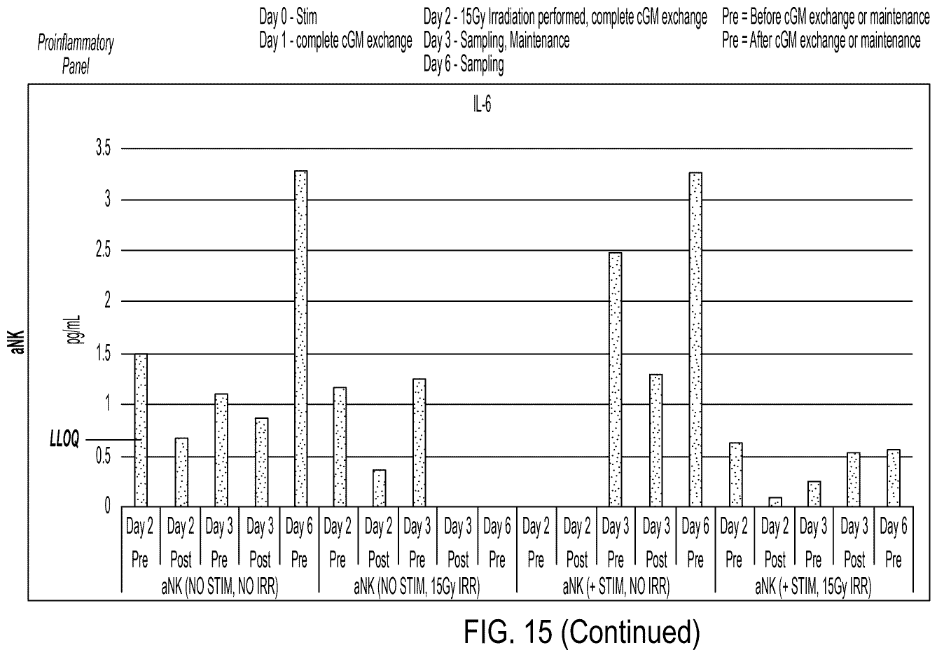

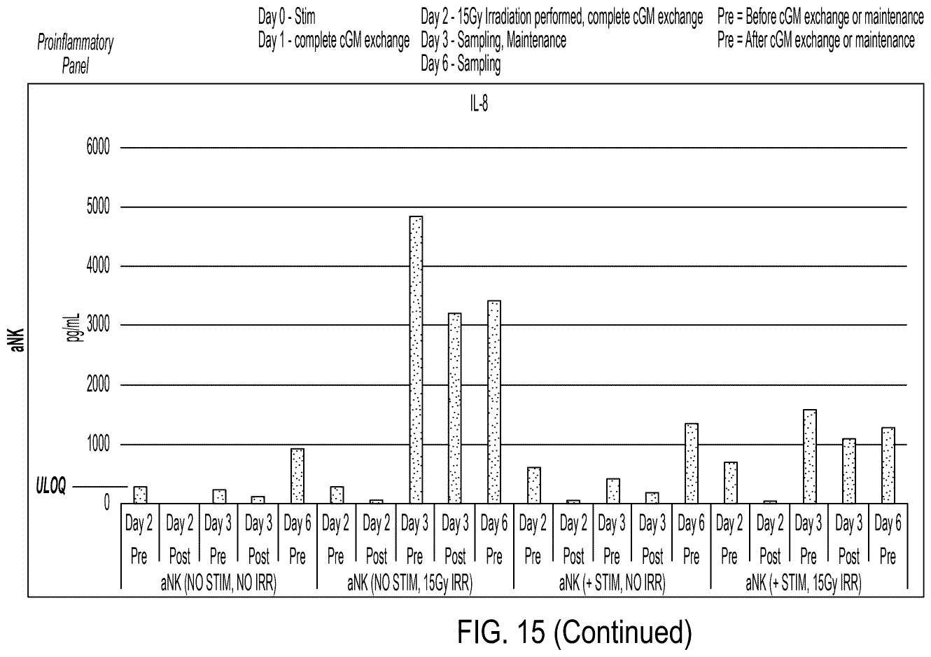

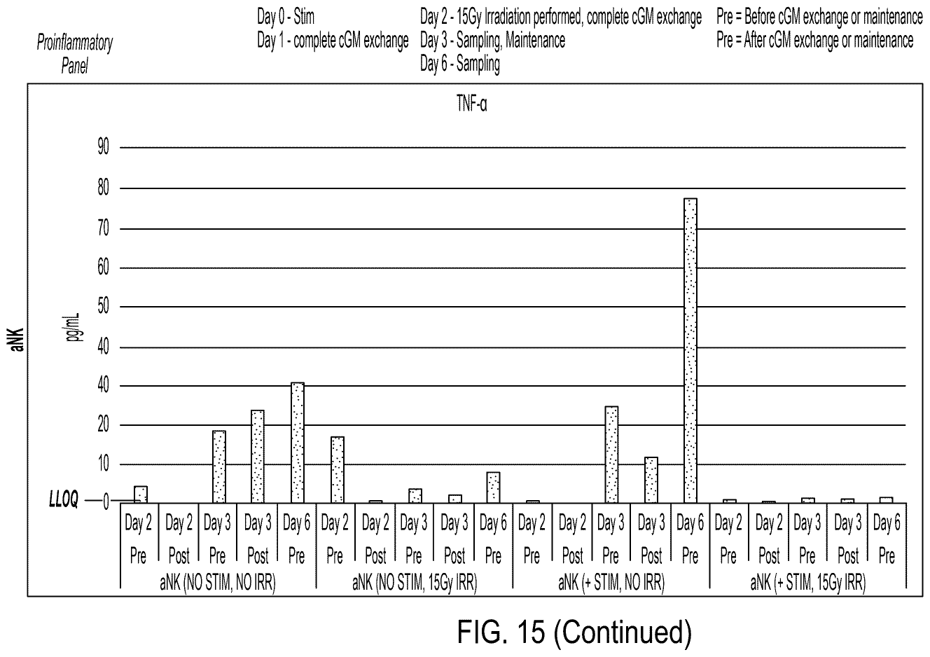

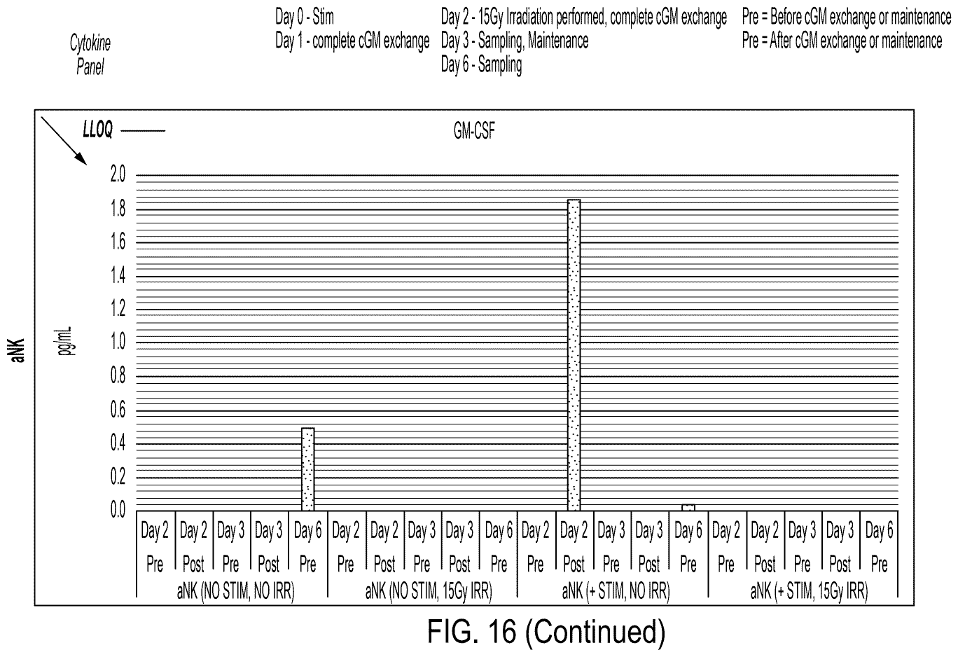

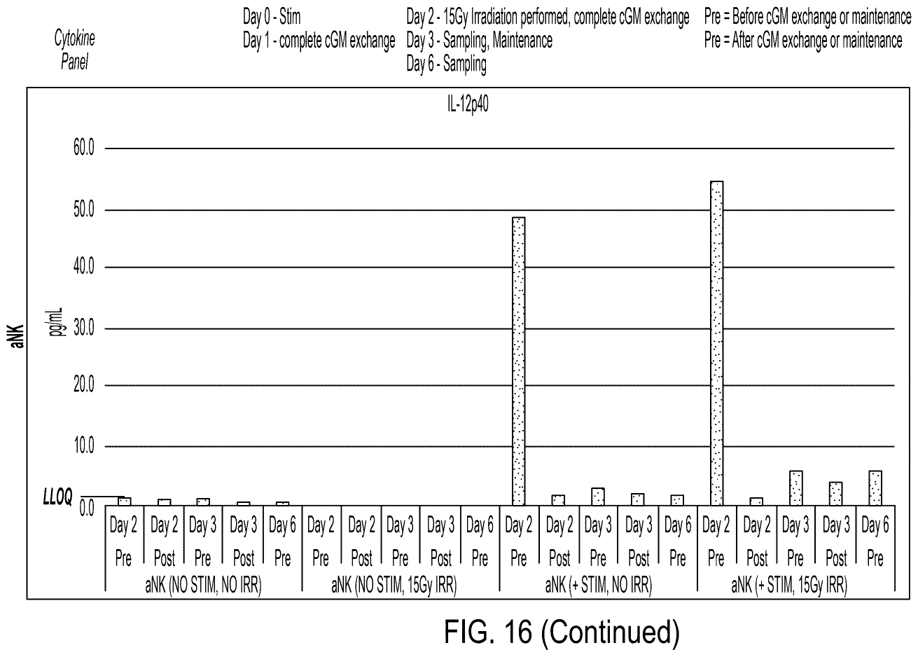

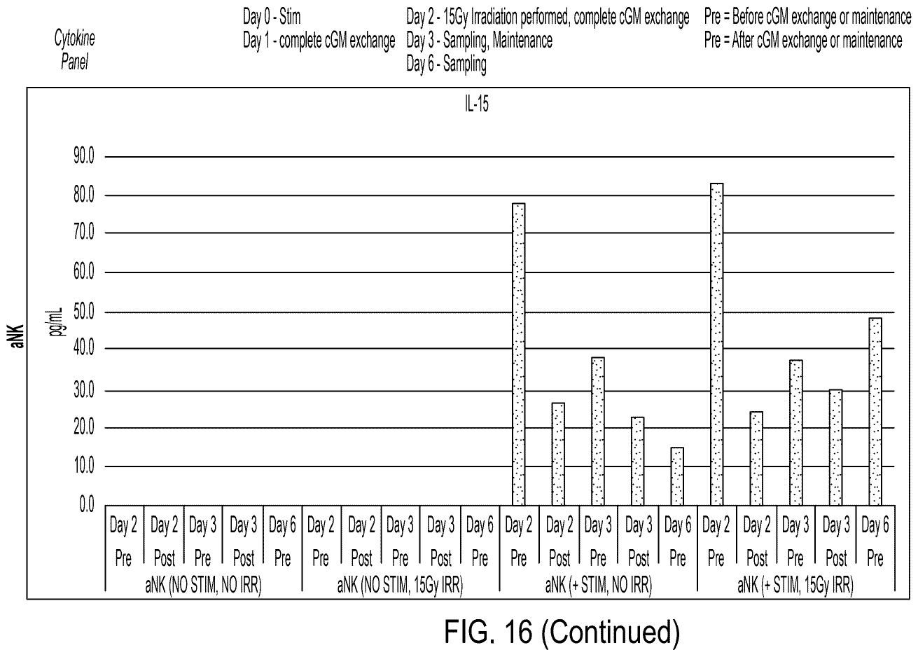

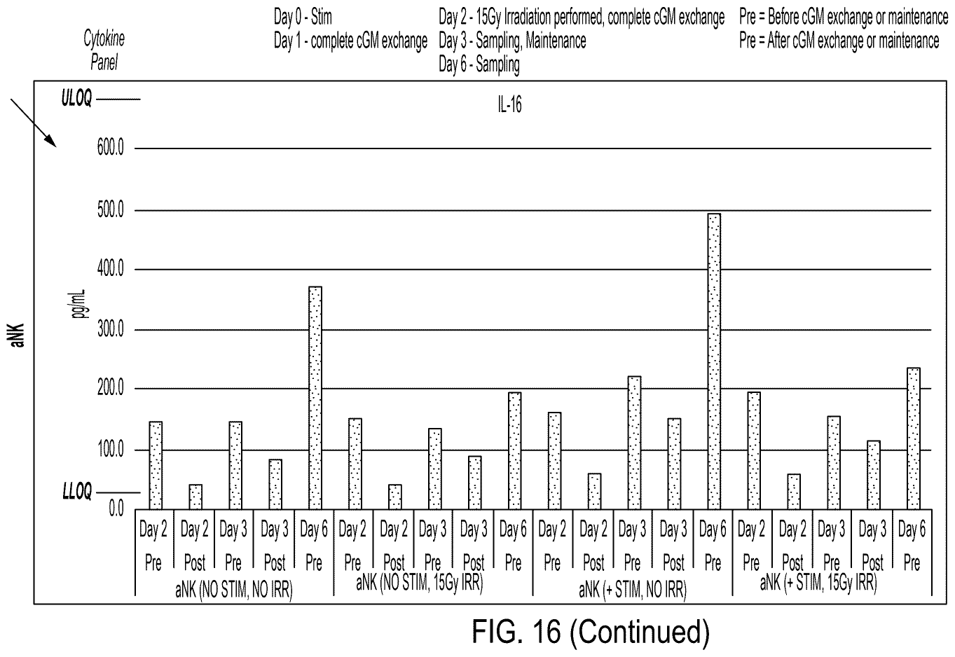

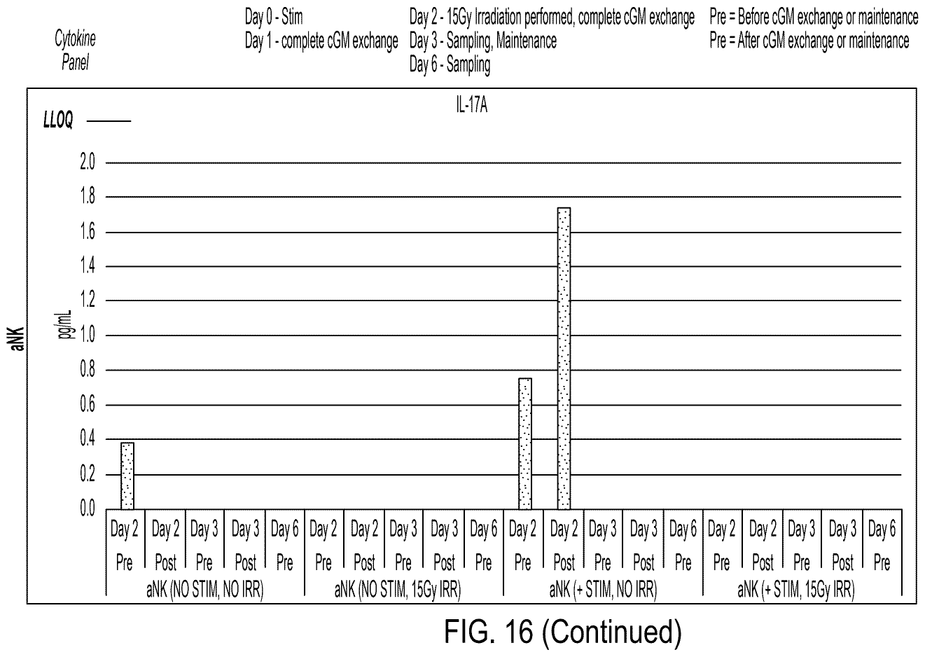

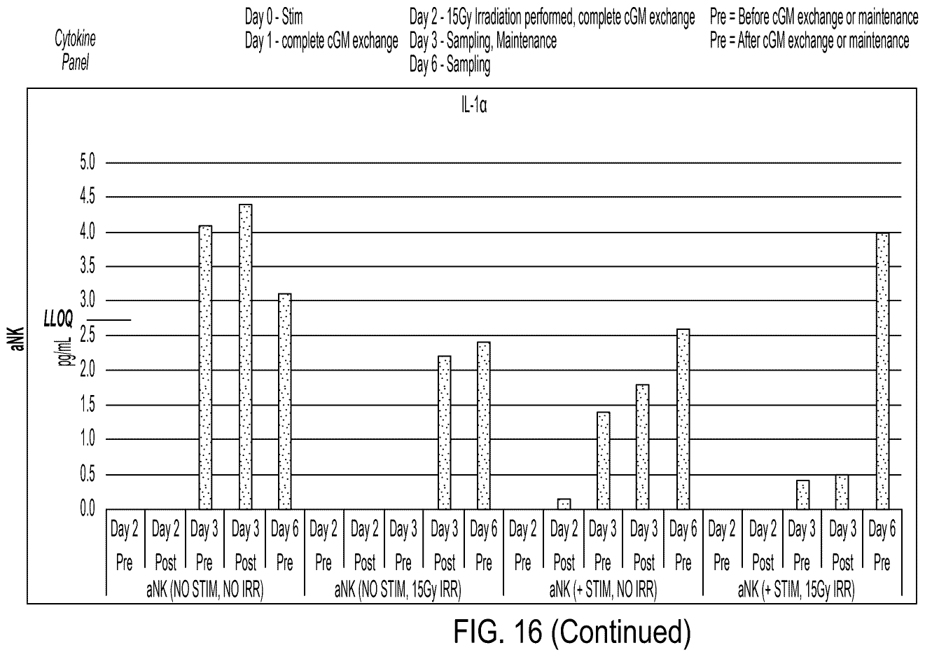

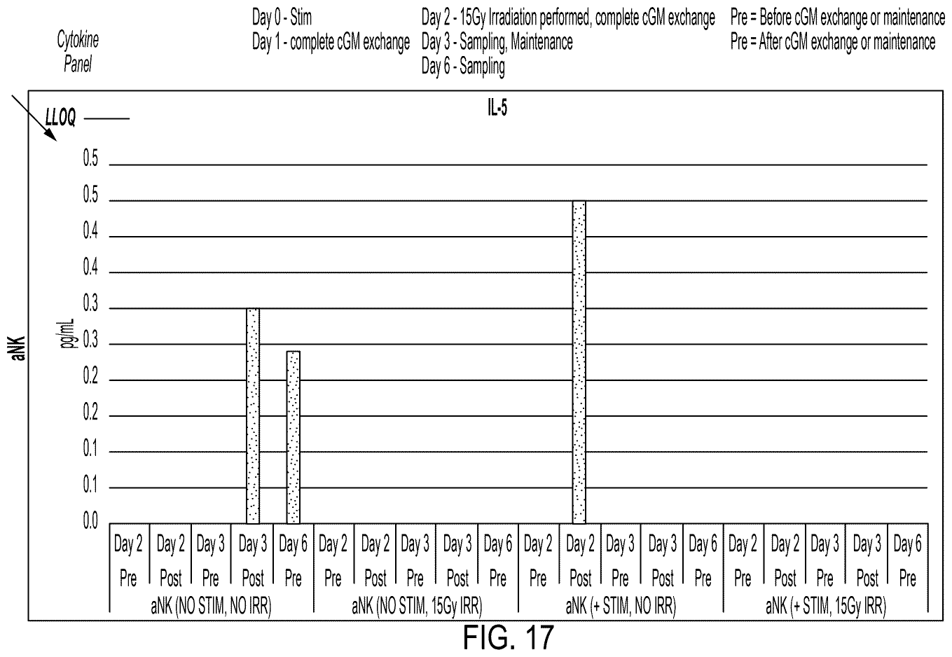

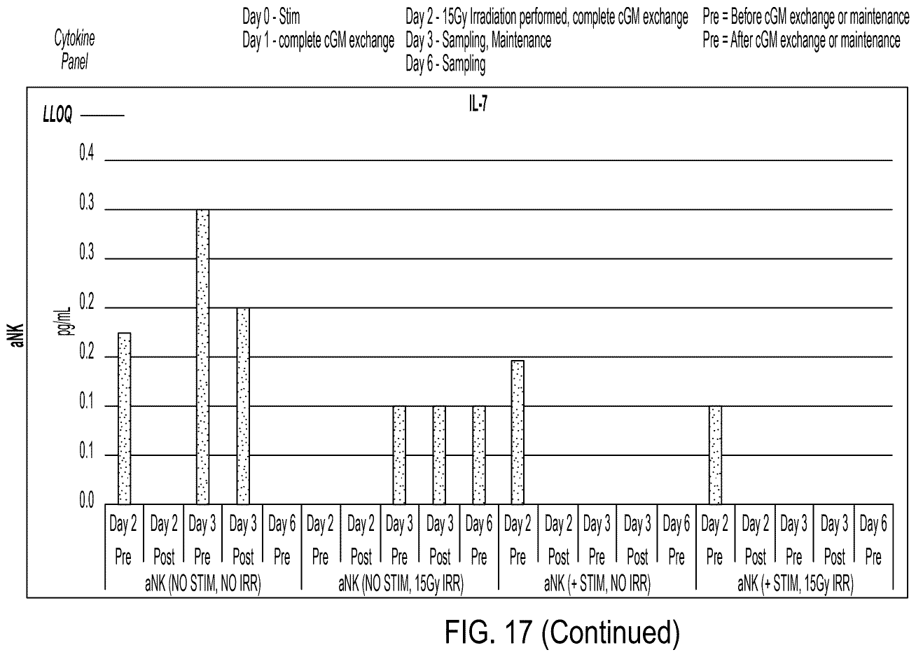

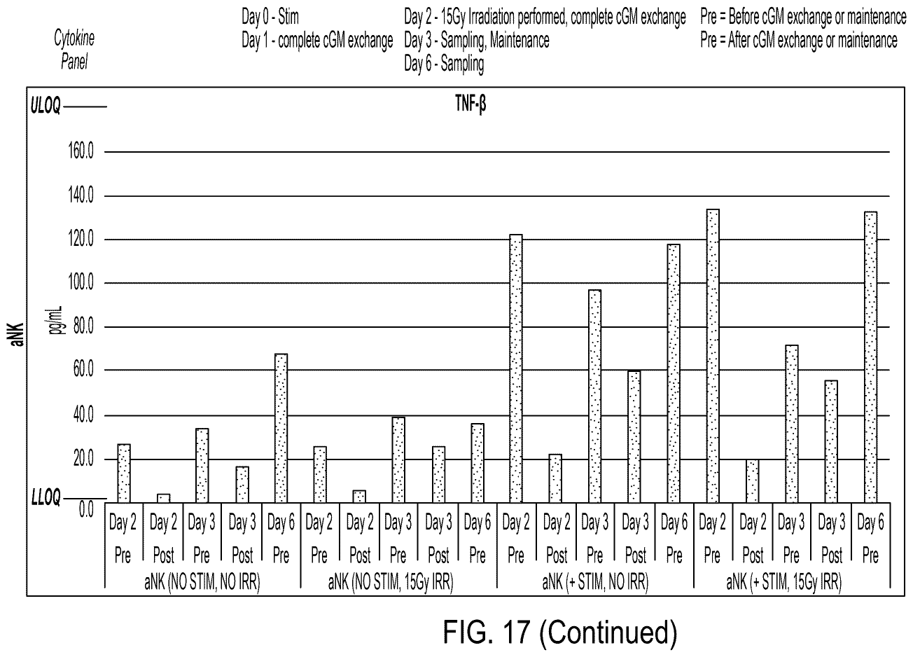

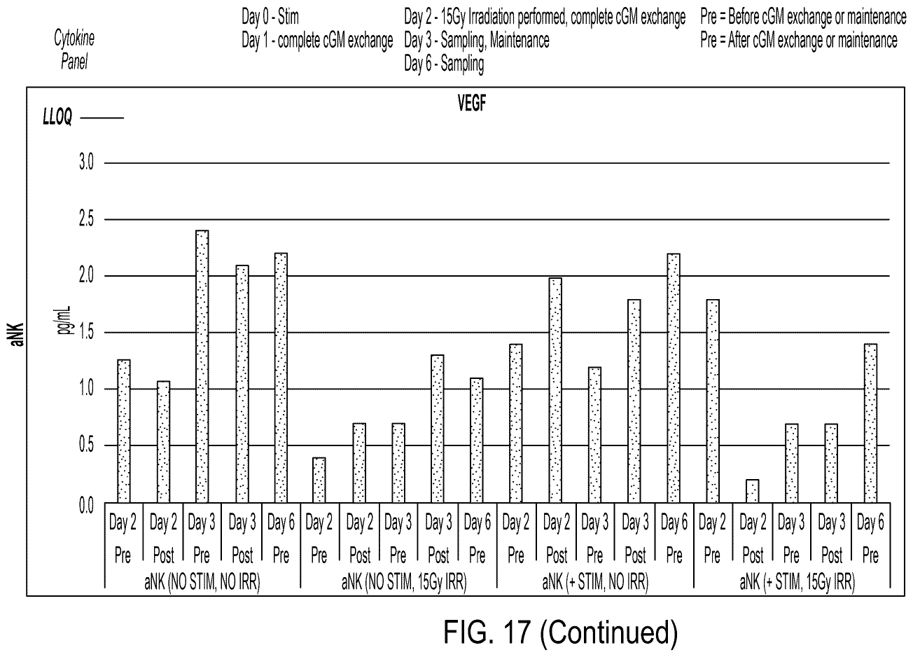

FIGS. 14-17 describe phenotypic profiling by mass cytometry: Samples taken 16 hours post-cytokine stimulation. Measured key NK surface and intracellular markers (CD25, IFNγ, GZMB, NCRs). Proleukin IL-2 spiked into iGM at every exchange or maintenance (final 450 IU/mL, total volume)

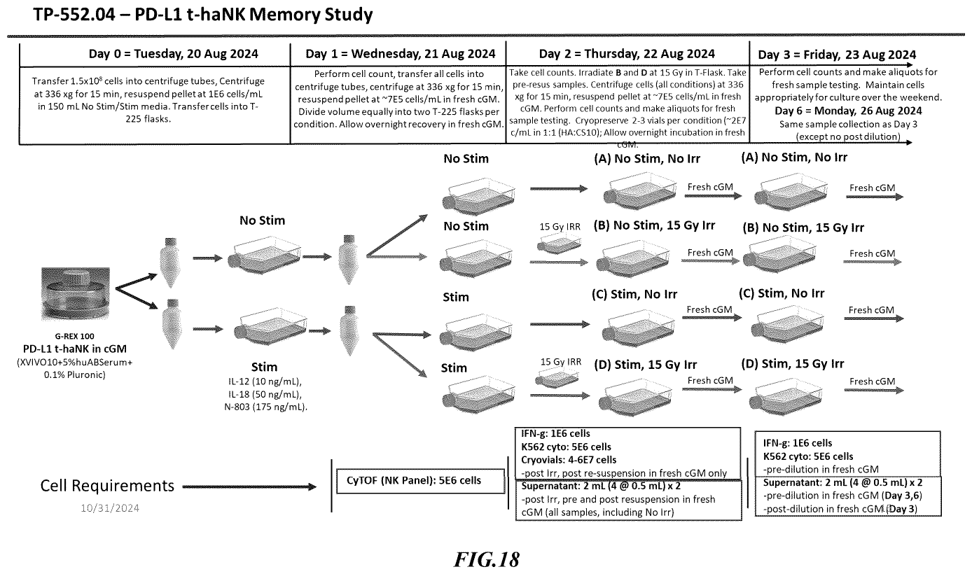

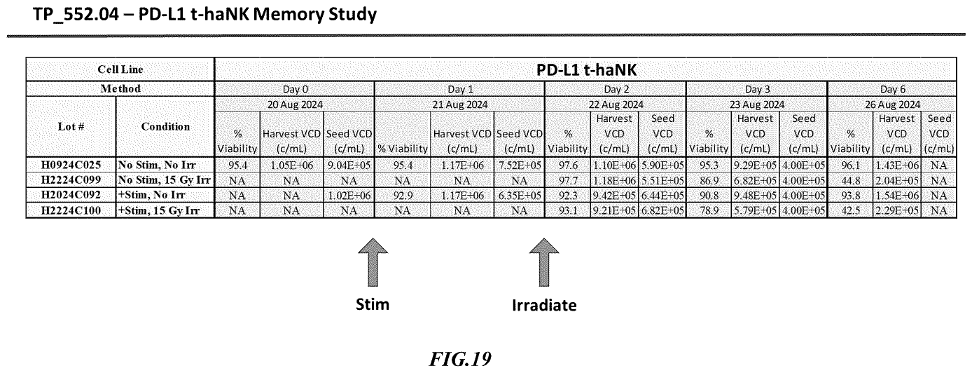

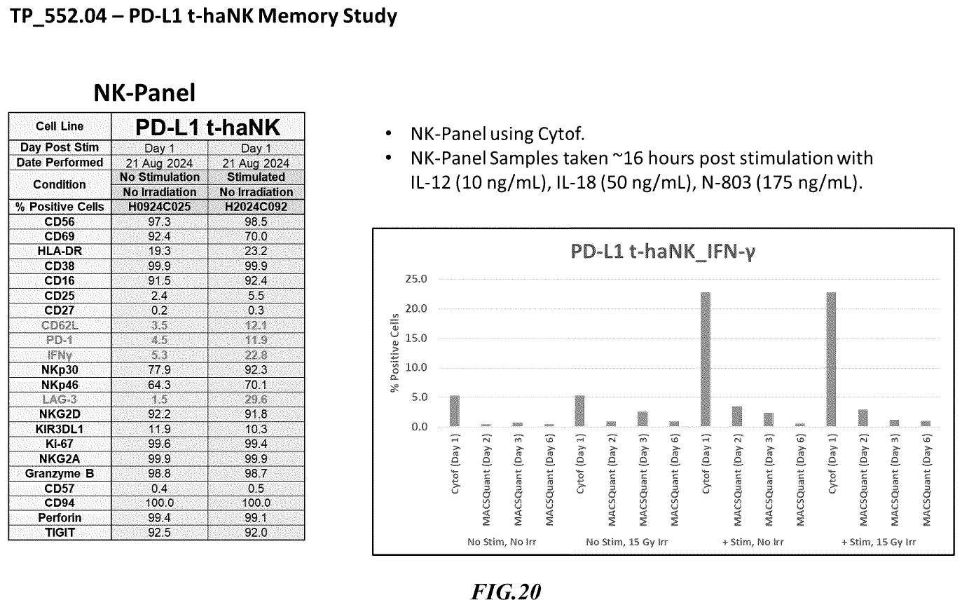

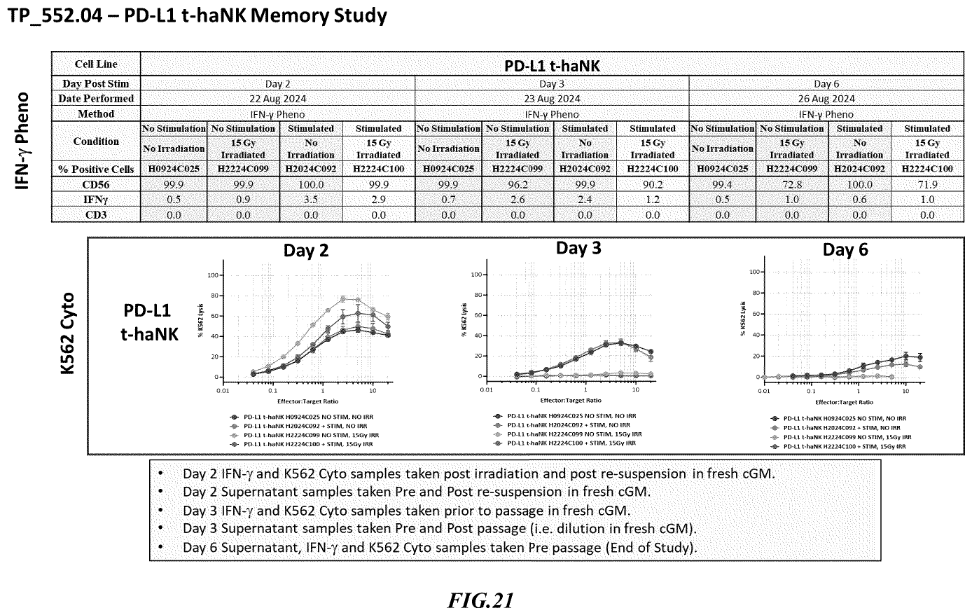

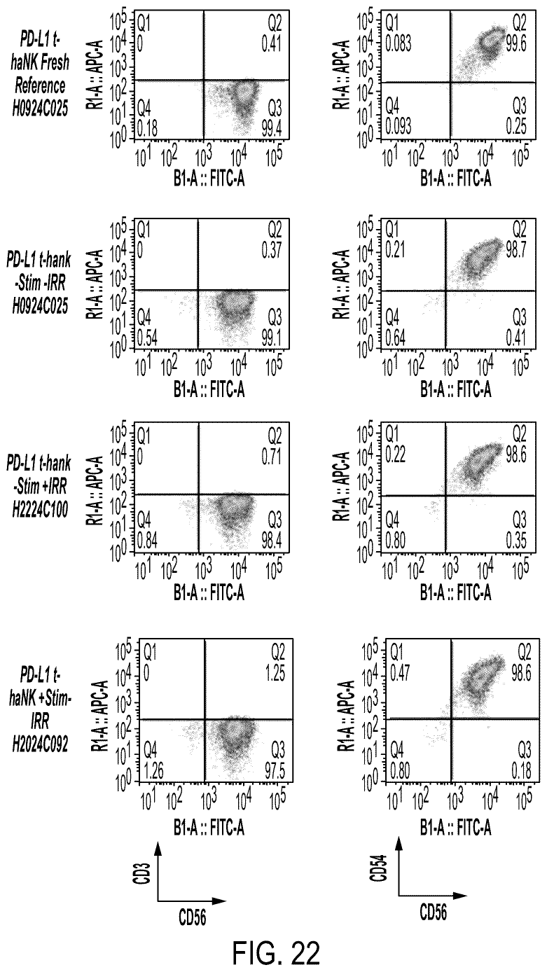

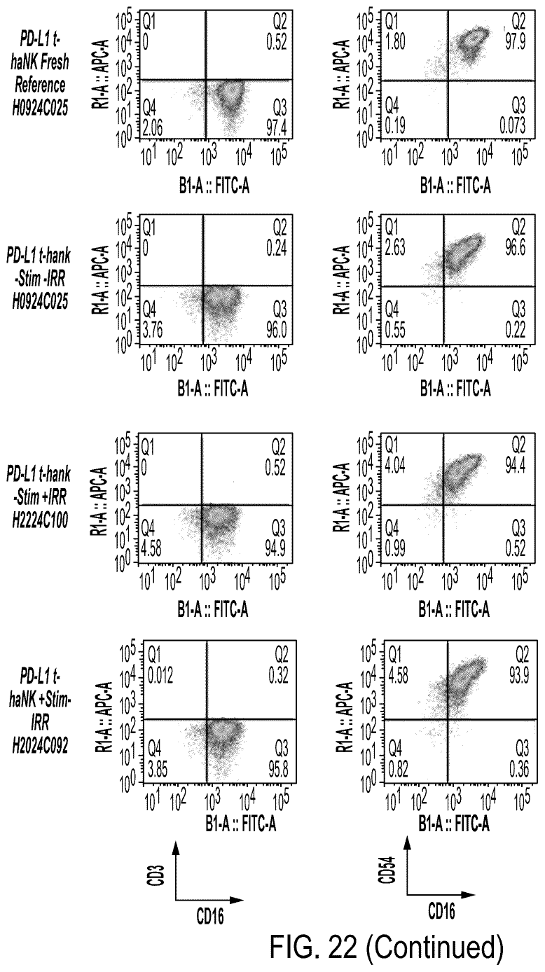

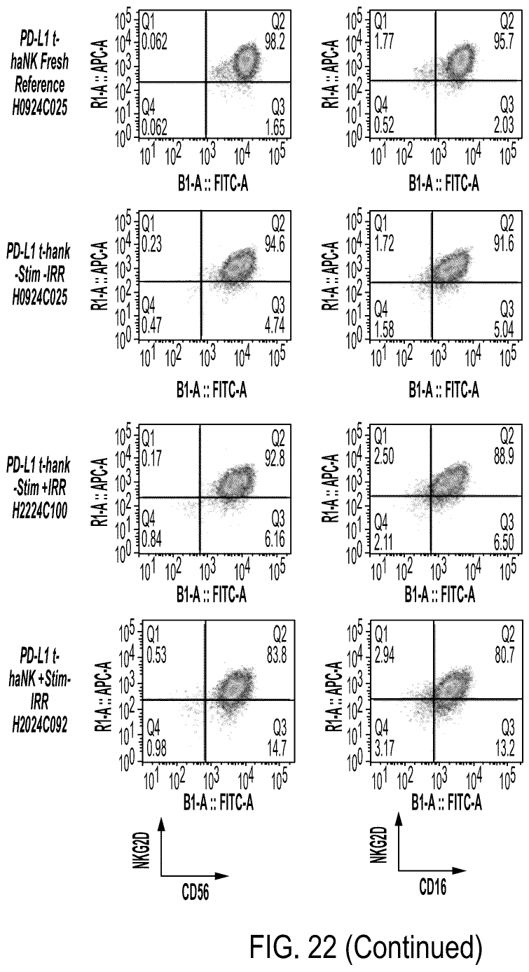

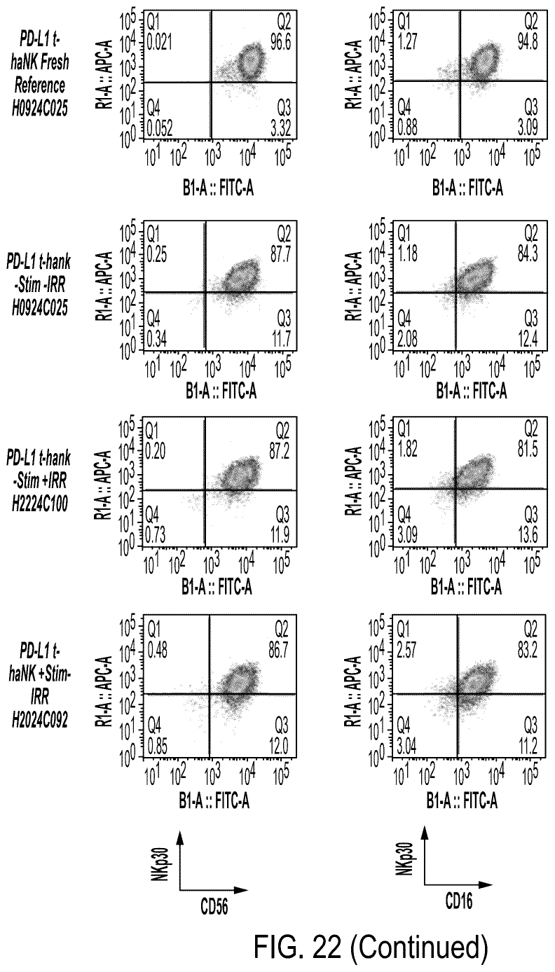

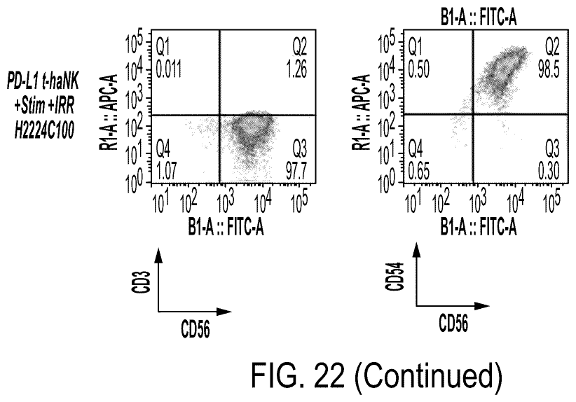

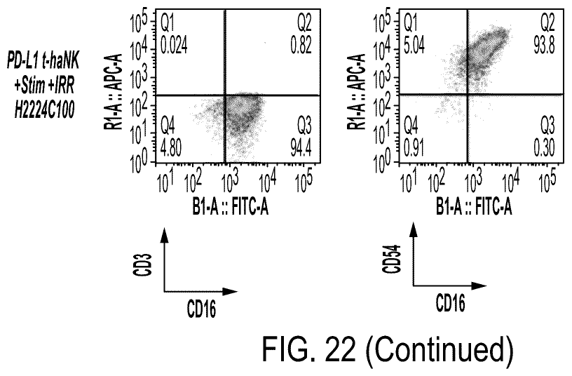

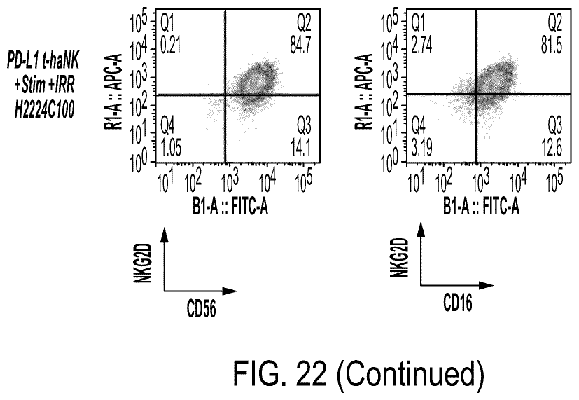

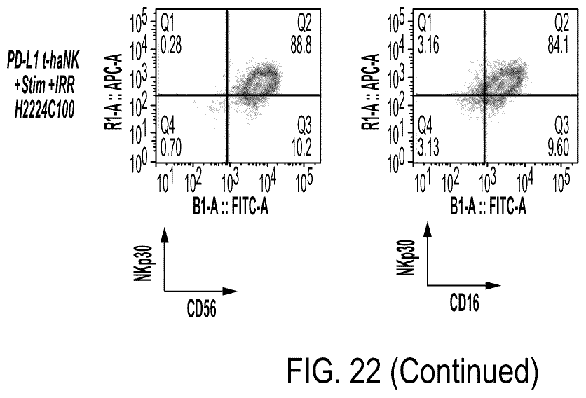

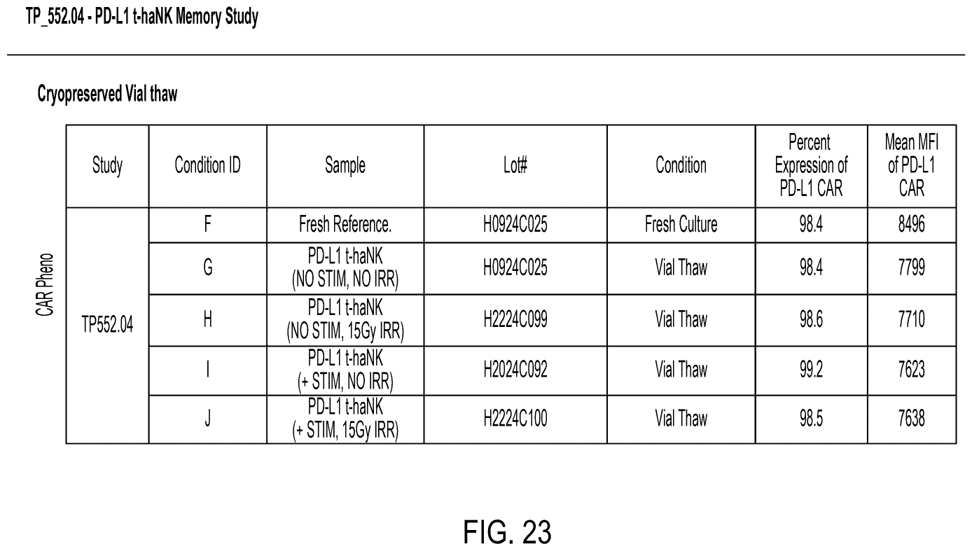

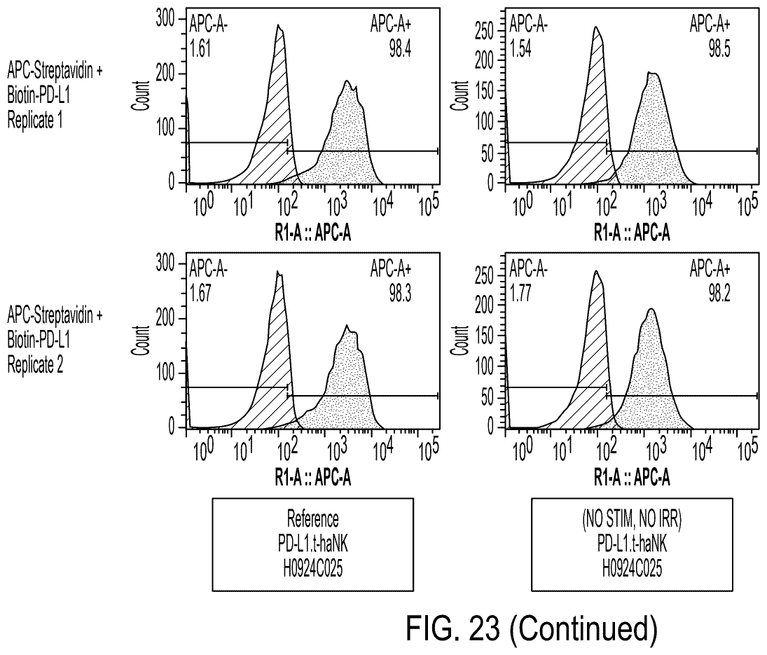

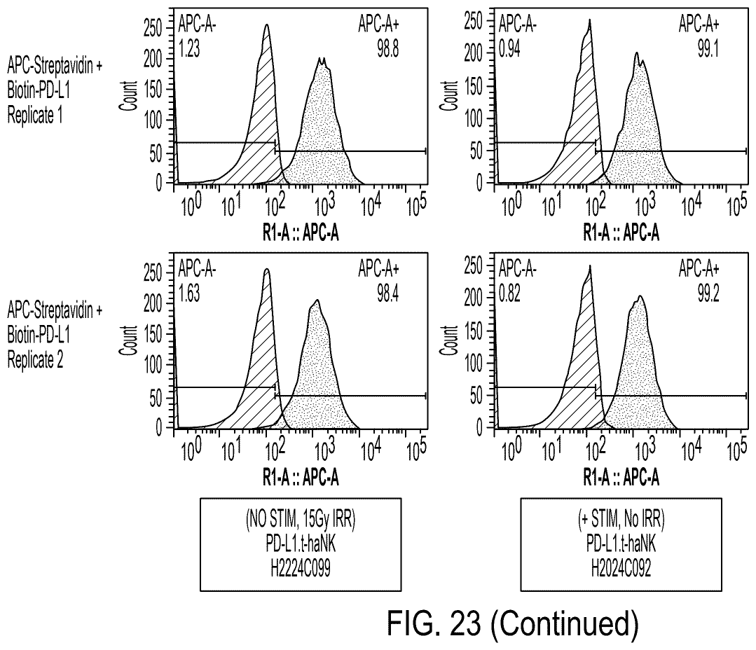

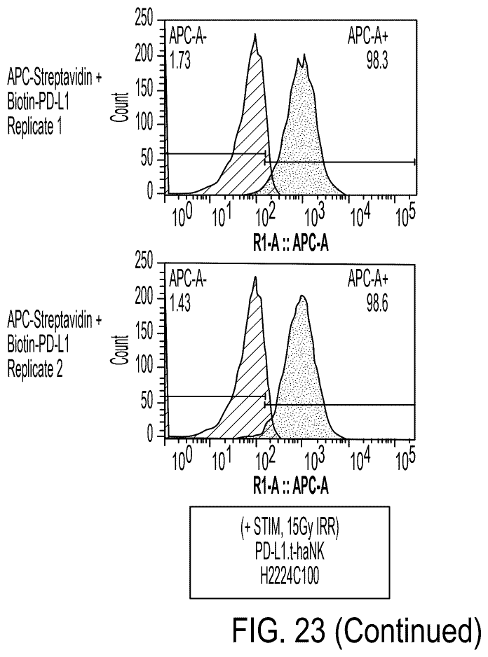

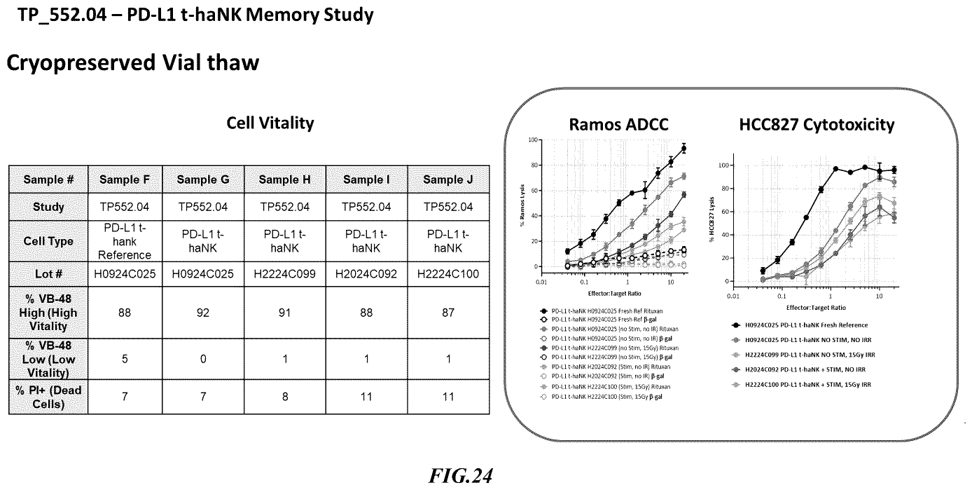

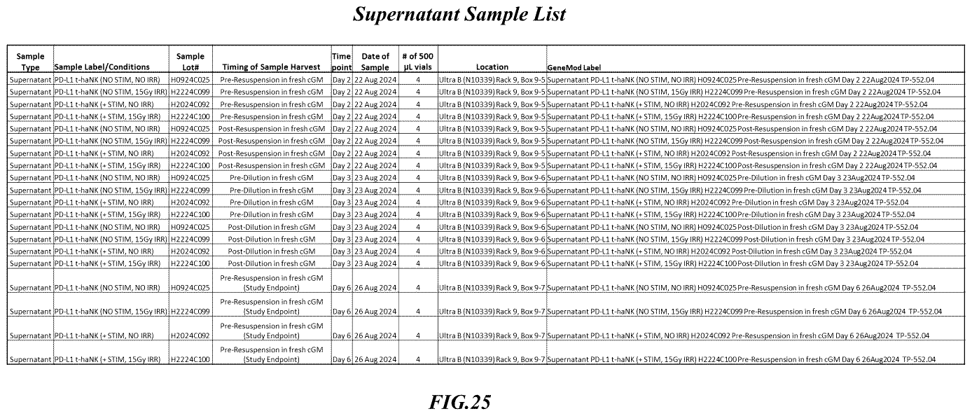

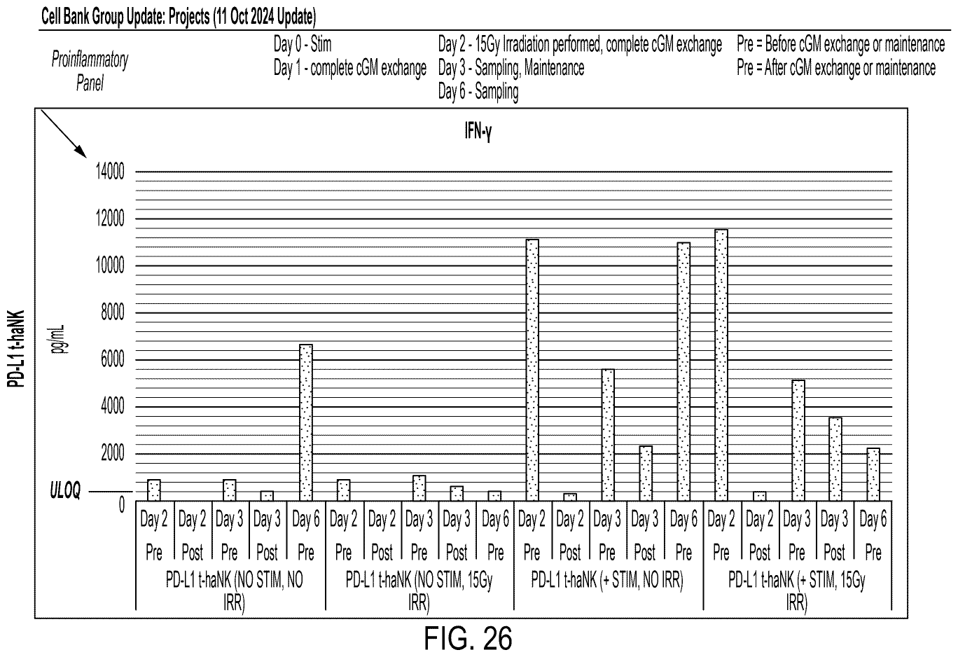

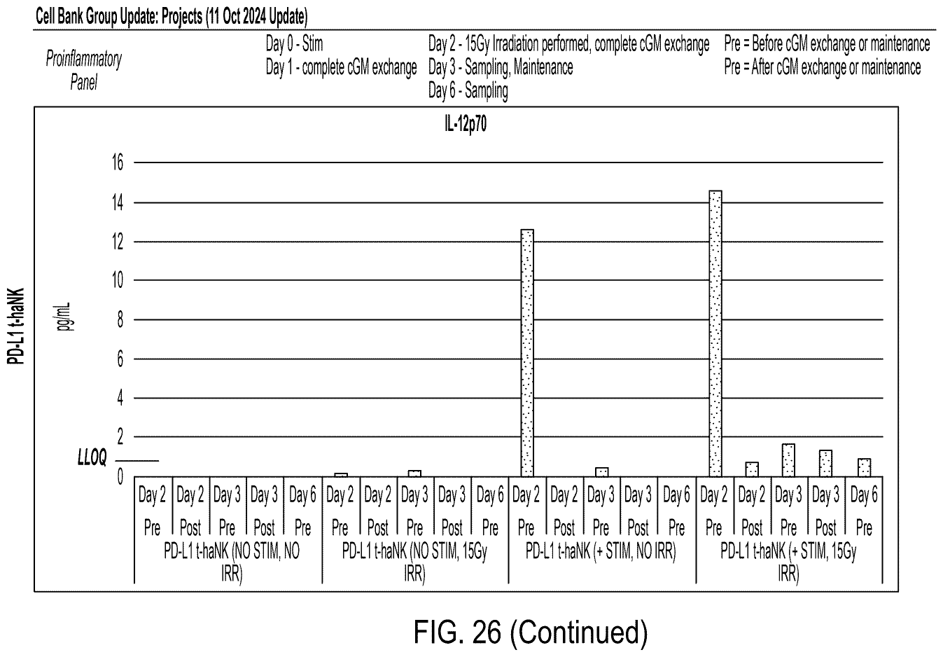

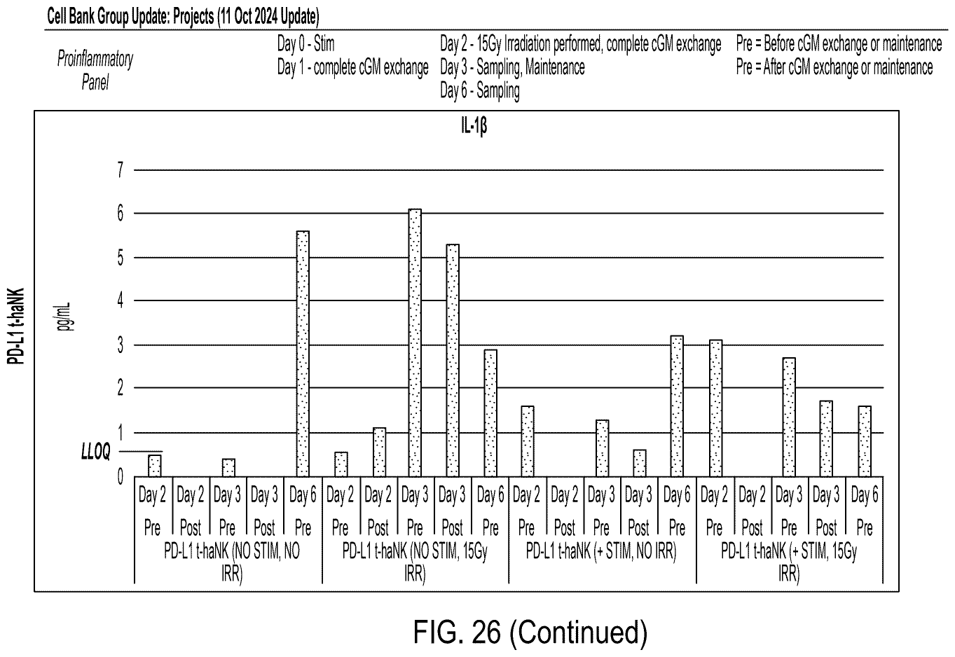

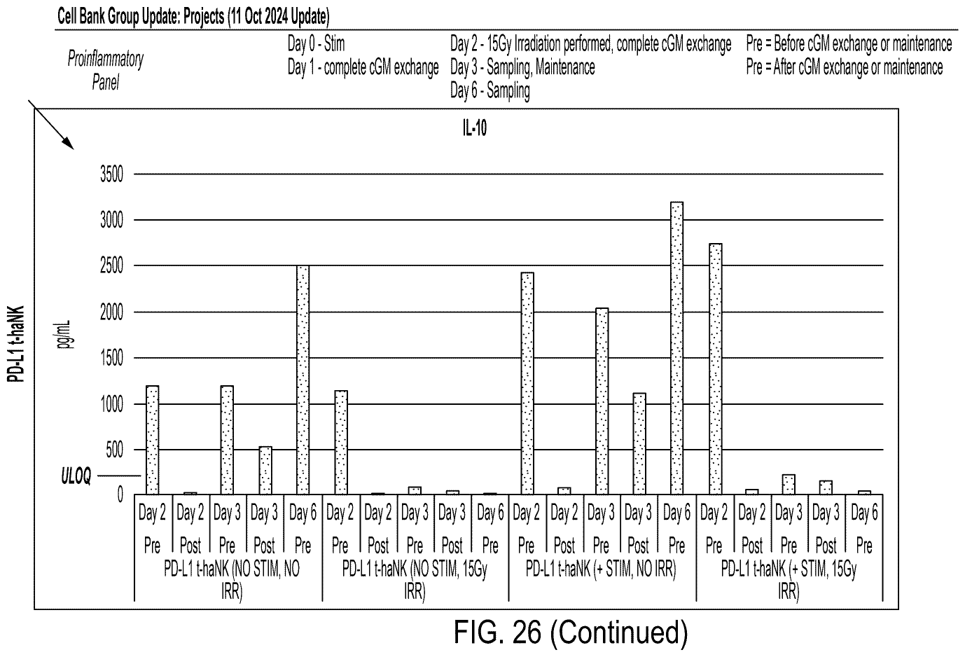

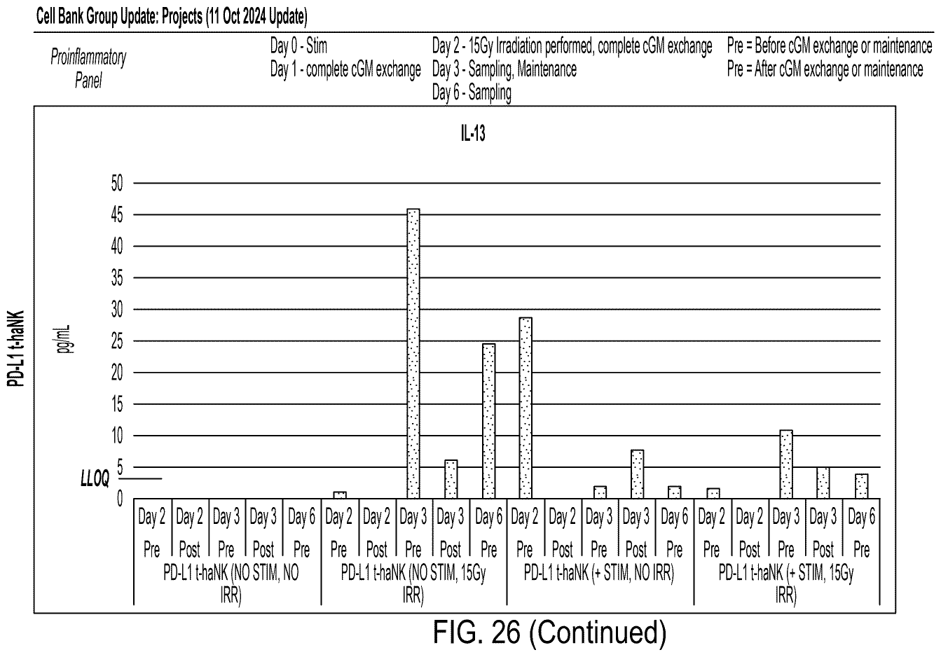

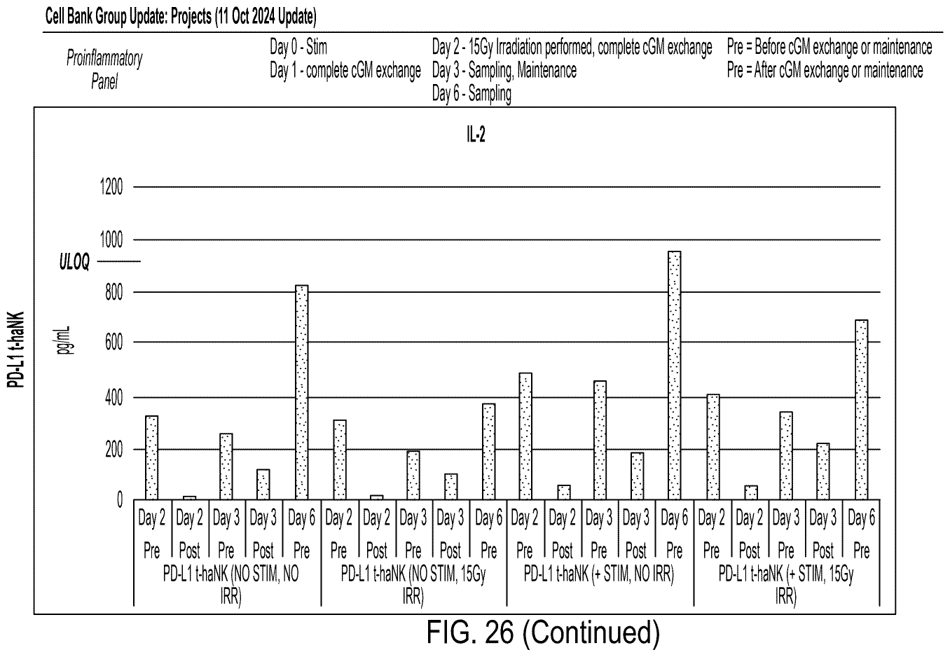

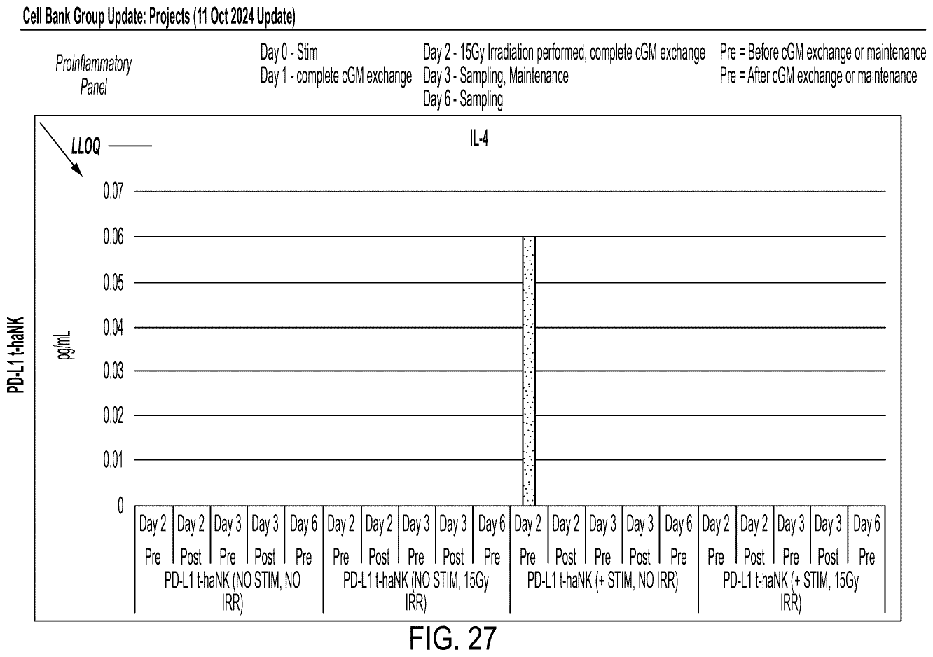

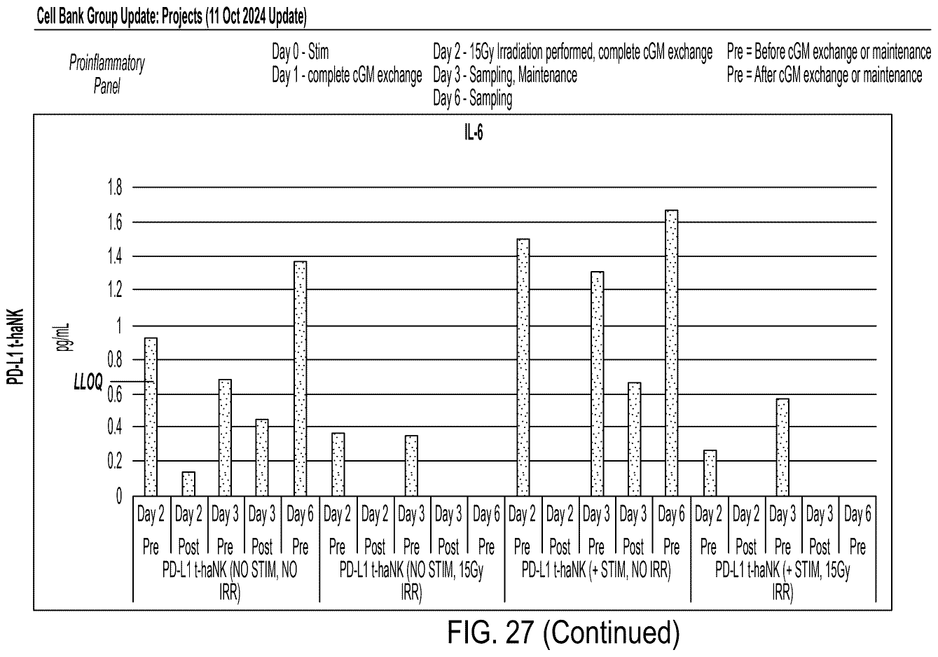

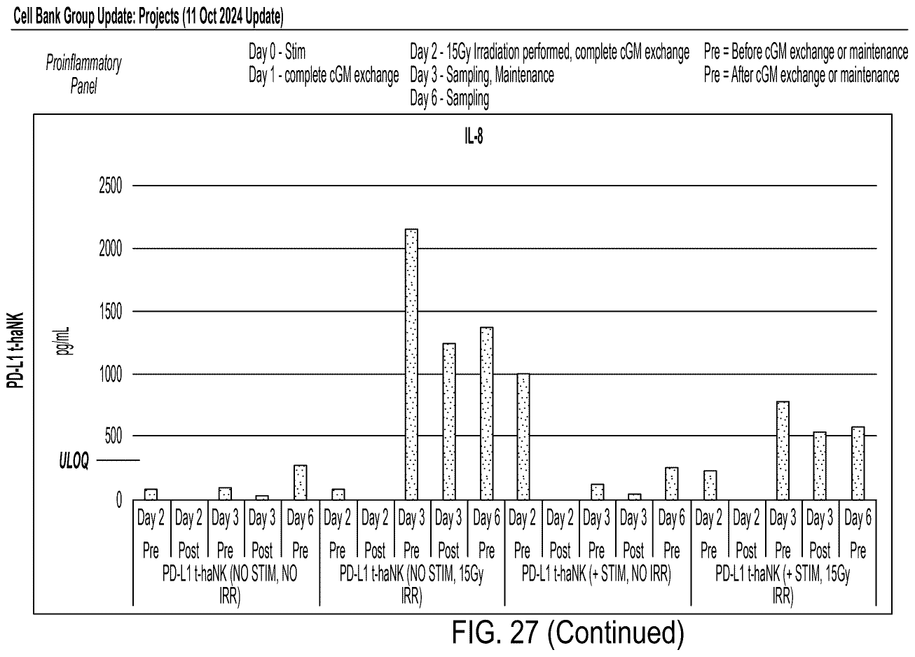

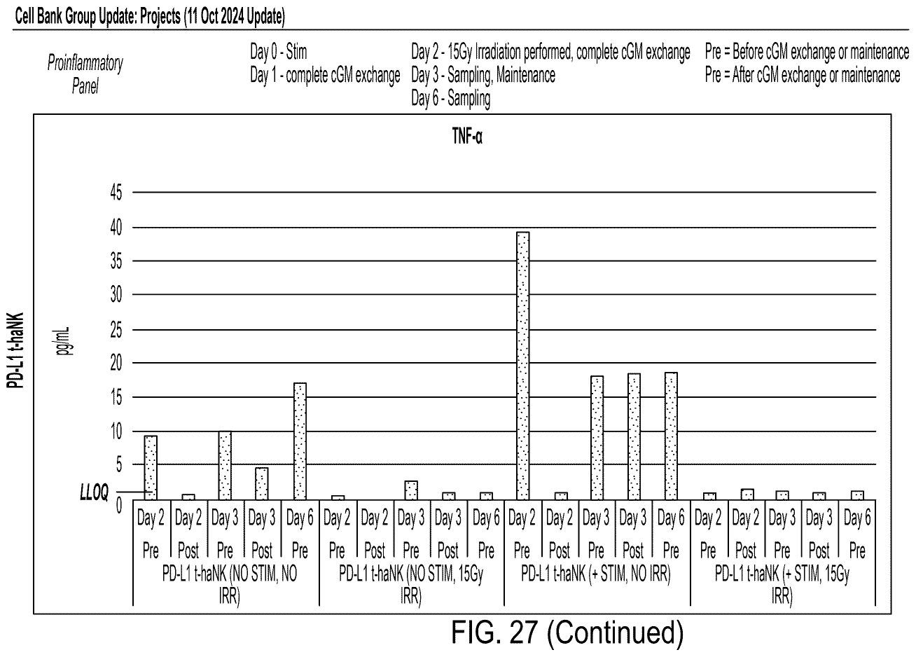

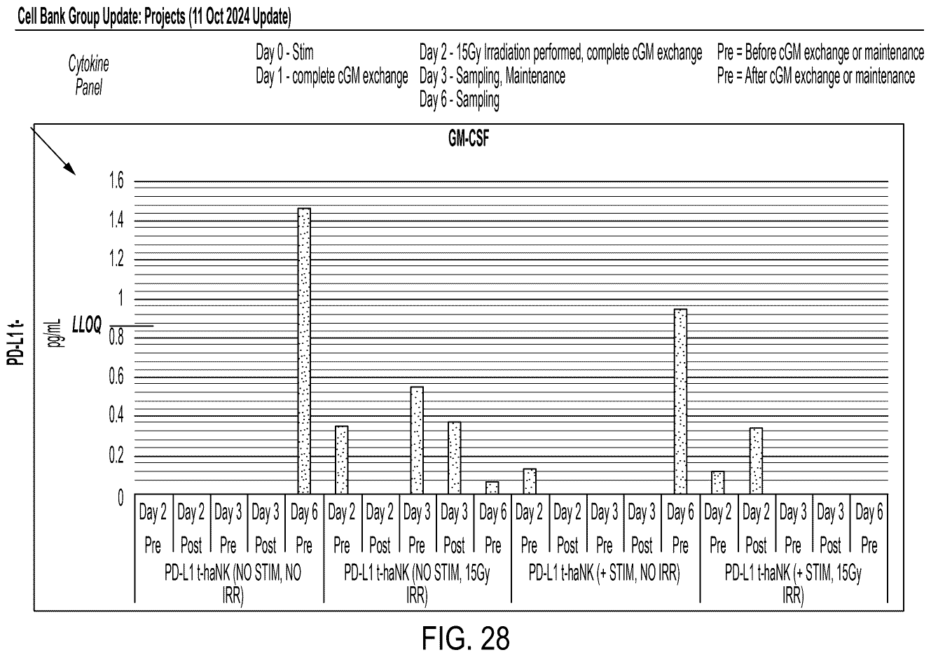

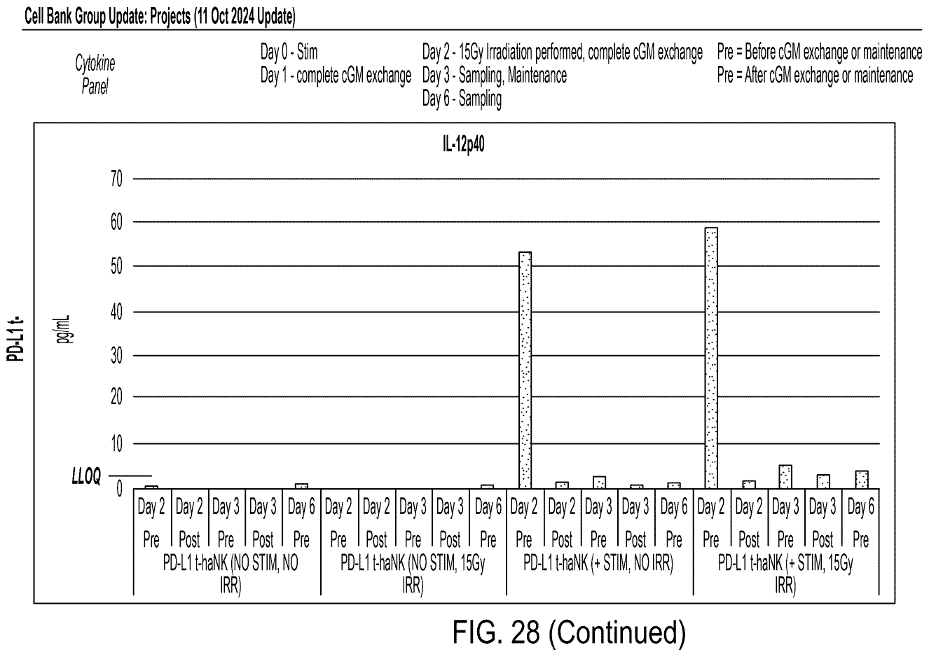

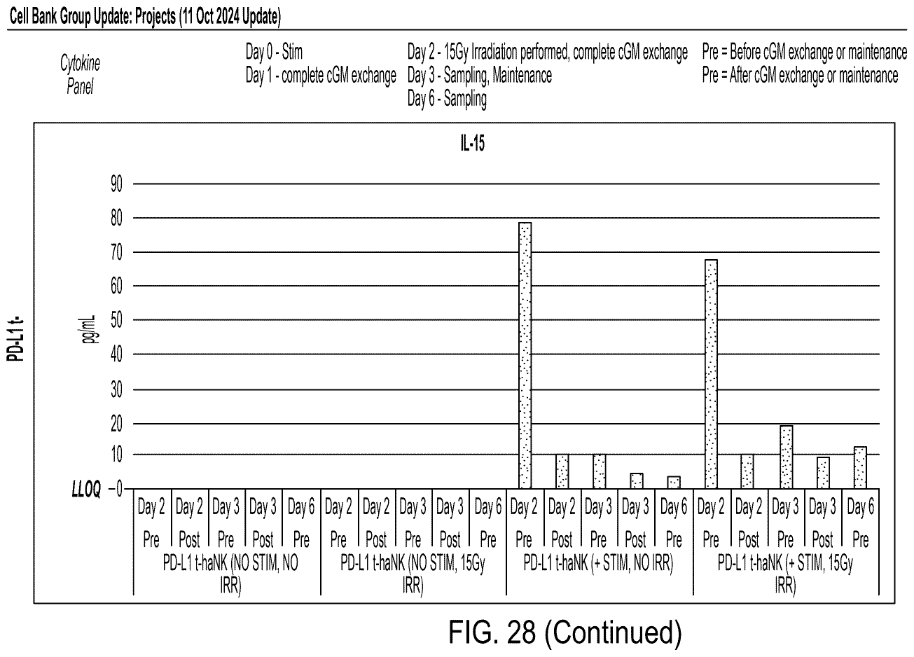

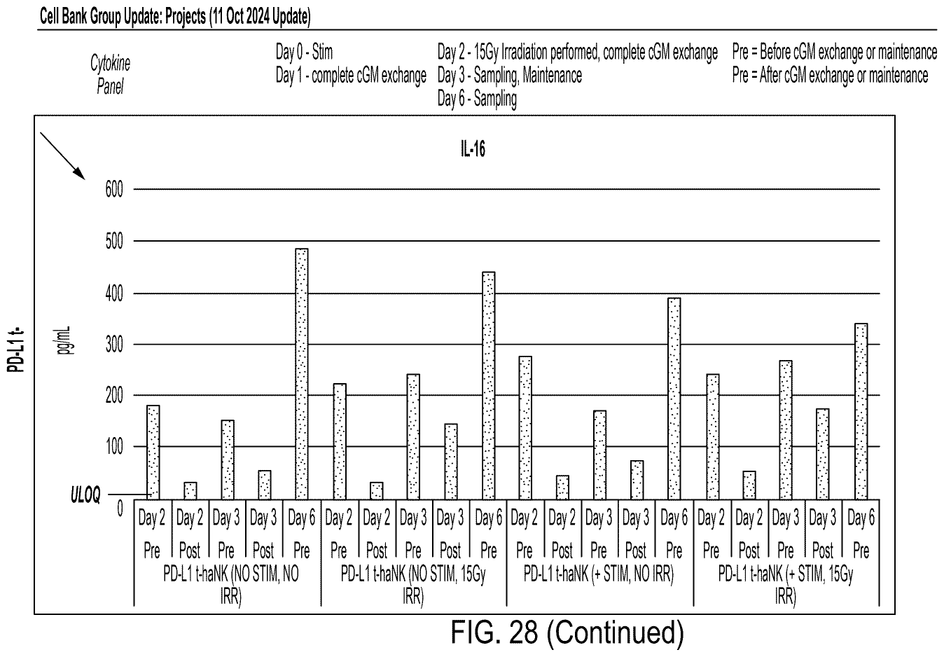

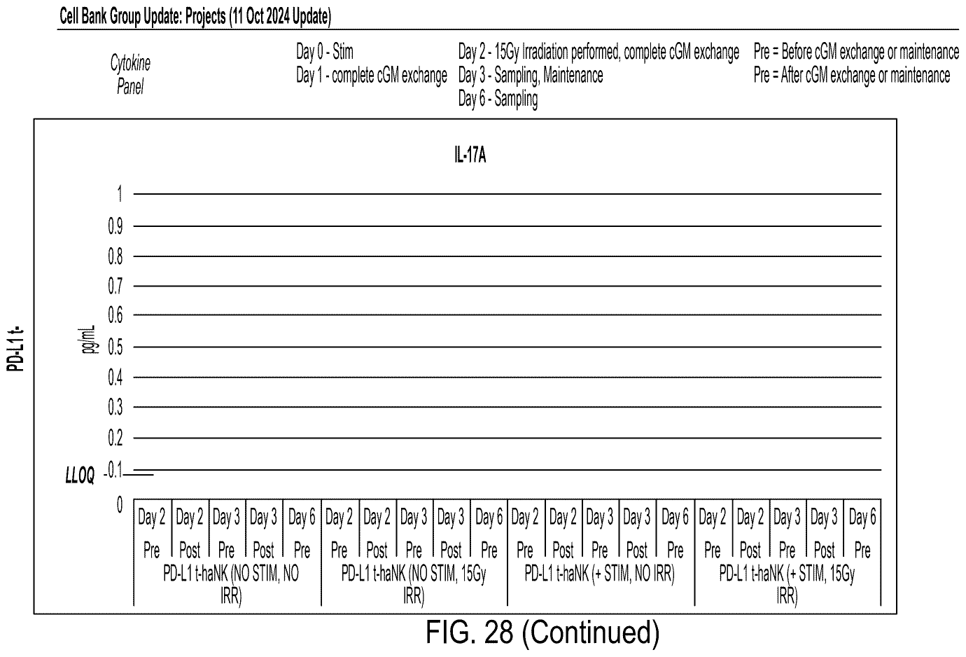

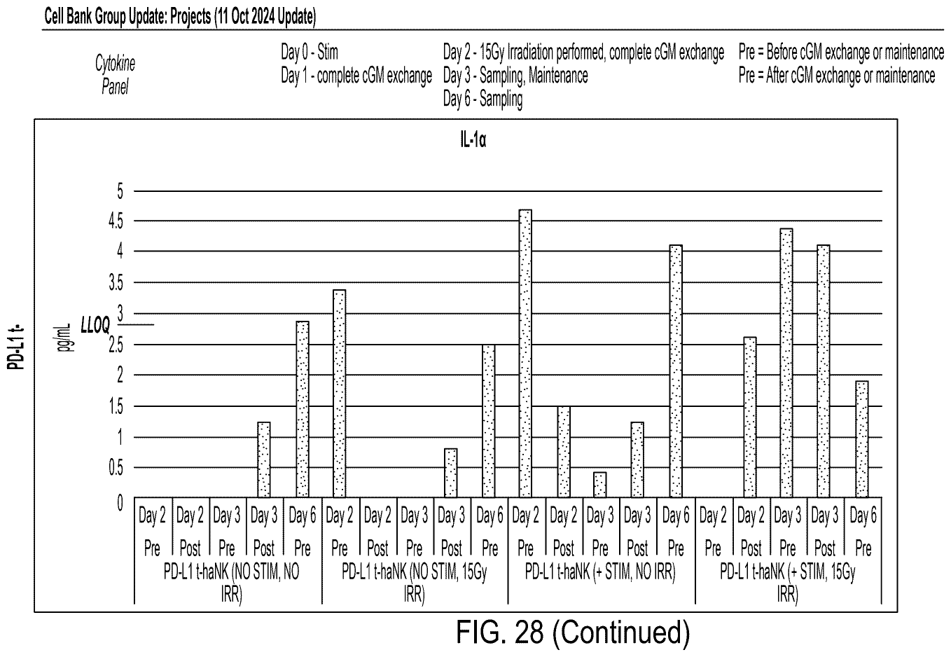

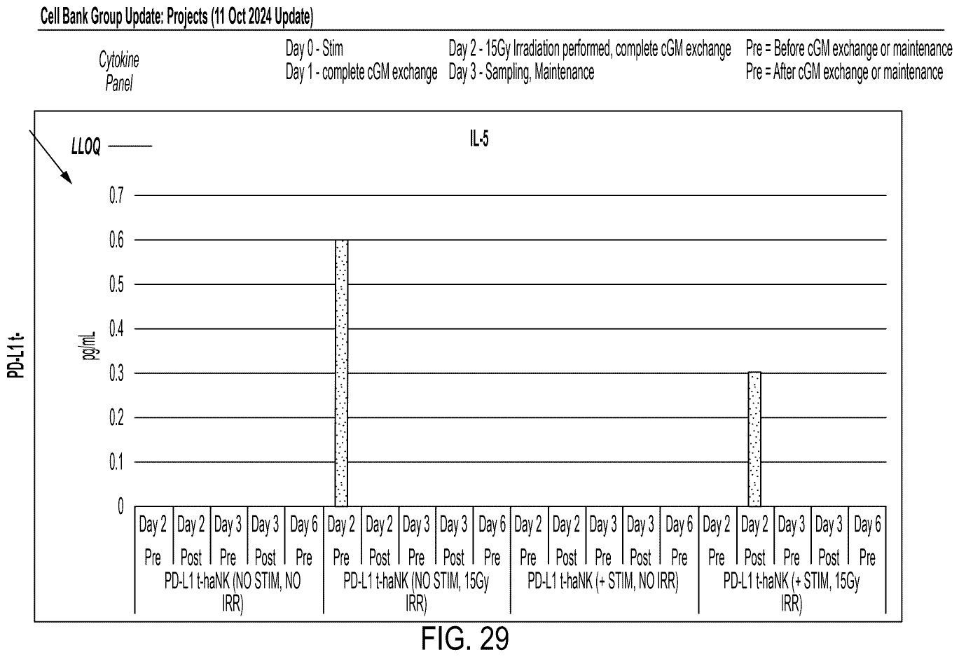

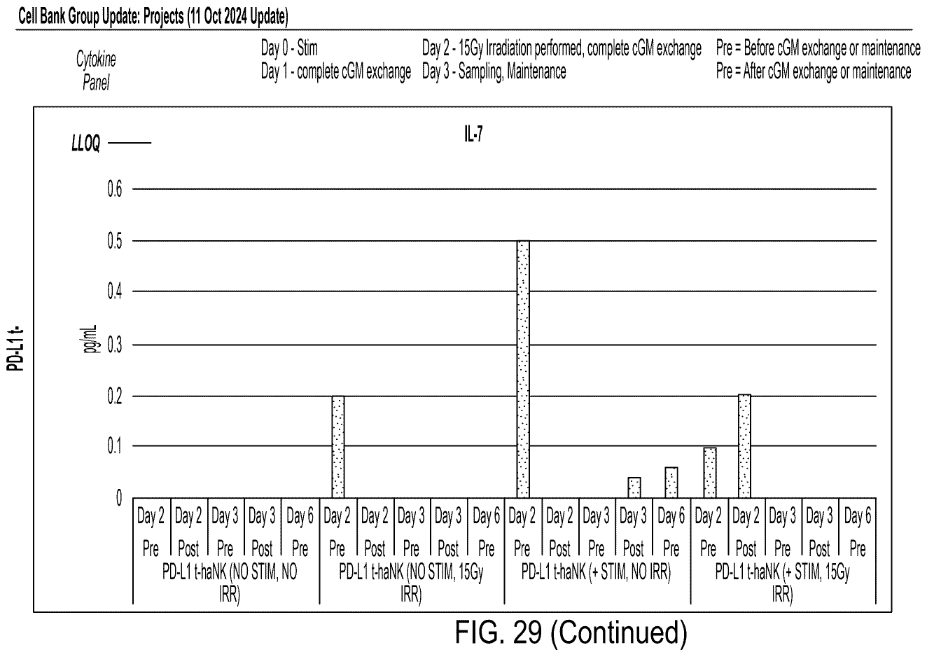

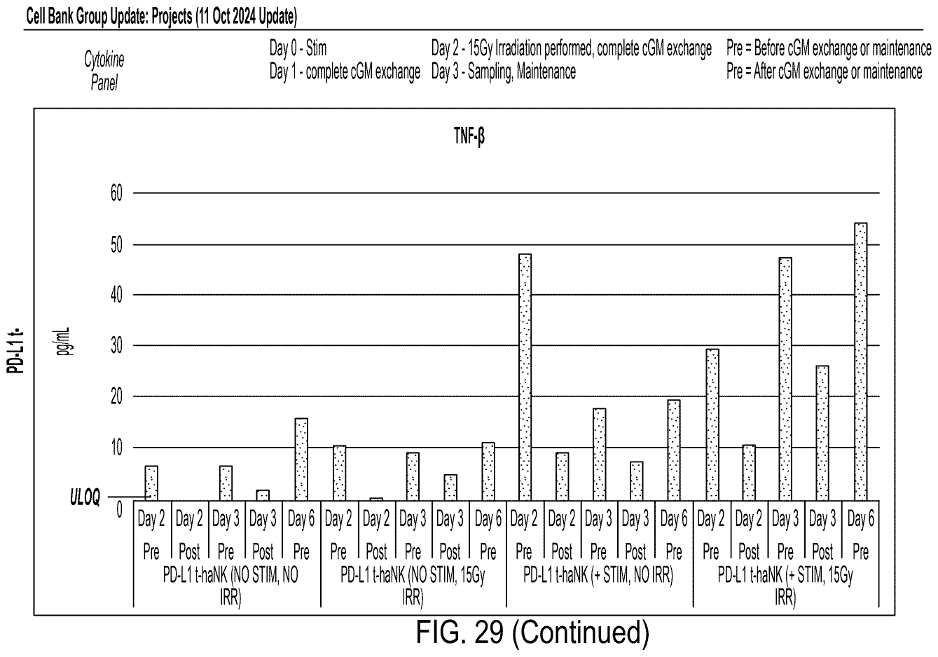

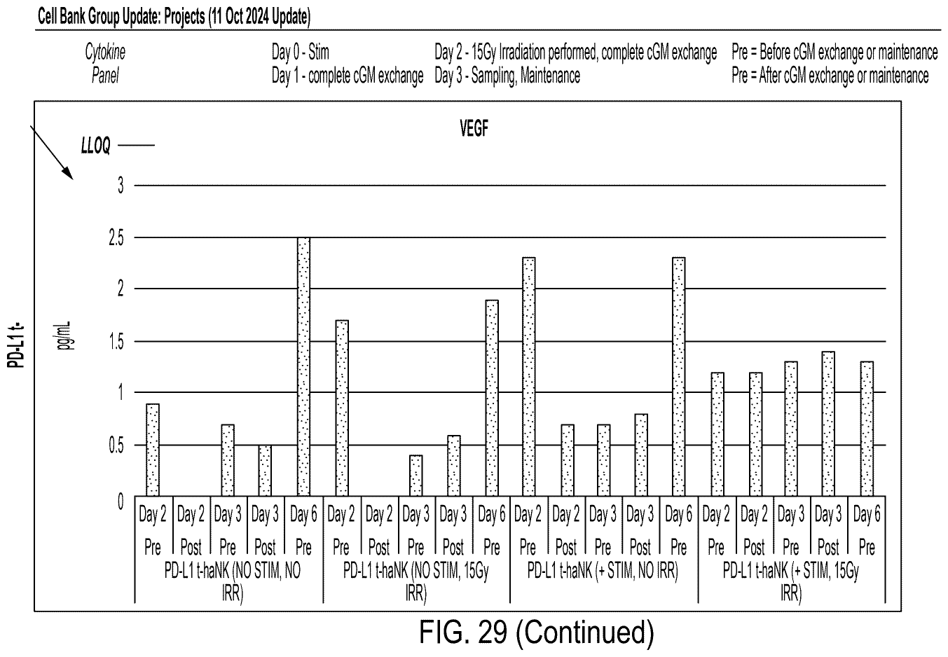

An identical workflow was applied to PD-L1 t-haNK cells to confirm that CAR expression does not interfere with memory induction. Cytokine stimulation and sampling followed the same timeline (Days 0-6). CyTOF panels (“CAR Pheno”) assessed PD-L1 CAR expression stability and IFNγ induction. IFNγ and cytotoxicity samples were collected at each time point (Day 2 post-irradiation, Day 3 pre- and post-passage, Day 6 pre-passage). The experimental protocol and results are shown in FIGS. 18-29 and Tables 4-5.

TABLE 4
Cryopreserved Cell Sample List

| | Label/ | | Timing of | | | # of vials | |
|---|---|---|---|---|---|---|---|
| | Conditions | Sample | Sample | | Date of | at 2E7 | |
| Sample Type | Sample | Lot# | Harvest | Timepoint | Sample | cells/vial | Location |
| Cryopreserved | PD-L1 t-haNK (NO | H0924C025 | Post- | Day 2 | 22 Aug. | 3 | Cryo B, Rack 7, |
| Cells | STIM, NO IRR) | | Resuspension in | | 2024 | | Box 7-1 |
| | | | fresh cGM | | | | |
| Cryopreserved | PD-L1 t-haNK (NO | H2224C099 | Post- | Day 2 | 22 Aug. | 3 | Cryo B, Rack 7, |
| Cells | STIM, 15Gy IRR) | | Resuspension in | | 2024 | | Box 7-1 |
| | | | fresh cGM | | | | |
| Cryopreserved | PD-L1 t-haNK | H2024C092 | Post- | Day 2 | 22 Aug. | 3 | Cryo B, Rack 7, |
| Cells | (+STIM, NO IRR) | | Resuspension in | | 2024 | | Box 7-1 |
| | | | fresh cGM | | | | |
| Cryopreserved | PD-L1 t-haNK | H2224C100 | Post- | Day 2 | 22 Aug. | 3 | Cryo B, Rack 7, |
| Cells | (+STIM, 15Gy IRR) | | Resuspension in | | 2024 | | Box 7-1 |
| | | | fresh cGM | | | | |

TABLE 5
Cryopreserved Vial thaw

| | Condition | | | Phenotype % Positive | | | | | |
|---|---|---|---|---|---|---|---|---|---|
| Study | ID | Lot # | Condition | CD3 | CD56 | CD16 | CD54 | NKG2D | NKp30 |
| TP552.04 | F | H0924C025 | PD-L1 t-haNK | 0.47 | 99.86 | 97.88 | 99.69 | 97.87 | 96.35 |
| | | | Fresh | | | | | | |
| | | | Reference | | | | | | |
| TP552.04 | G | H0924C025 | PD-L1 t-haNK − | 0.32 | 99.33 | 96.61 | 99.07 | 94.08 | 86.72 |
| | | | Stim − IRR | | | | | | |
| TP552.04 | H | H2224C099 | PD-L1 t-haNK − | 0.62 | 99.03 | 95.21 | 98.63 | 92.19 | 85.36 |
| | | | Stim + IRR | | | | | | |
| TP552.04 | I | H2024C092 | PD-L1 t-haNK + | 0.79 | 98.68 | 94.67 | 98.93 | 83.99 | 86.48 |
| | | | Stim − IRR | | | | | | |
| TP552.04 | J | H2224C100 | PD-L1 t-haNK + | 1.06 | 98.89 | 94.28 | 98.92 | 84.58 | 88.17 |
| | | | Stim + IRR | | | | | | |

As shown in FIGS. 1-29, the culture supernatants were analyzed for cytokine content using multiplex immunoassays. Both memory-like aNK and memory-like PD-L1 t-haNK cultures exhibited selective up-regulation of IFNγ with limited induction of other proinflammatory cytokines (e.g., TNFα, IL-6), confirming that the response was targeted rather than globally inflammatory.

It should be recognized that both aNK and PD-L1 t-haNK memory-like lines were irradiated, tested, and cryopreserved under cGMP-compliant procedures, supporting scalability for clinical manufacture.

Based on the data and results presented here, the instant disclosure provides a mechanistic model comprising (i) an induction phase, where a brief exposure of NK-92-derived cells to IL-12, IL-18, and N-803 drives transcriptional reprogramming toward a memory-like state characterized by sustained IFNγ production and CD25 expression; (ii) a recall phase, where upon encountering target cells, memory-like cells release elevated IFNγ in a localized manner; (iii) a feedback sensitization, where IFNγ induces PD-L1 expression on neighboring tumor cells, converting “cold” tumors to “hot” immunologic states; (iv) selective killing, where PD-L1 t-haNK cells recognize and eliminate PD-L1 positive tumor cells via CAR-mediated engagement, creating an amplifying cytotoxic loop. This system enables tumor-localized IFNγ delivery without systemic cytokine exposure, reducing the risk of cytokine-related toxicities while enhancing tumor clearance. The described memory-like PD-L1 t-haNK cell line combines the persistence and recall features of CIML-NK biology with the precision of CAR targeting.

In some embodiments, the numbers expressing quantities of ingredients, properties such as concentration, reaction conditions, and so forth, used to describe and claim certain embodiments of the invention are to be understood as being modified in some instances by the term “about.” As used herein, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus, the value to which the modifier “about” or “approximately” refers is itself also specifically disclosed. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

1. A method of treatment for a patient with cancer, the method comprising:
exposing NK-92 cells to a medium comprising a cytokine cocktail, wherein the cytokine cocktail comprises individual cytokines or fusion proteins comprised thereof, wherein the cocktail comprises IL-15 or N-803, IL-12, and IL-18;
removing the cytokine cocktail from the medium; and
administering a therapeutically effective amount of the NK-92 cells to the patient.

2. The method of claim 1, wherein the NK-92 cells are exposed to the cytokine cocktail for 12 hours, 24 hours, 36 hours, 48 hours, or 96 hours.

3. The method of claim 1, wherein the NK-92 cells are cryopreserved after removing the cytokine cocktail.

4. The method of claim 1, wherein the NK-92 cells are administered 1 day, 2 days, 3 days, 4 days, 5 days, 6 days, 7 days, or more than 7 days after removing the cytokine cocktail.

5. The method of claim 1, further comprising engineering the NK-92 cells to express erIL-2 and CD16.

6. The method of claim 1, further comprising engineering the NK-92 cells to express a Chimeric Antigen Receptor (CAR).

7. The method of claim 6, wherein the CAR comprises an extracellular binding domain having specific binding affinity for a tumor or viral antigen.

8. The method of claim 7, wherein the extracellular binding domain is specific for PD-L1, EGFR, HER2, CD19, B7-H4, or BCMA.

9. The method of claim 1, wherein the cytokine cocktail comprises N-803.

10. The method of claim 1, wherein the cytokine cocktail comprises HCW-9201.

11. The method of claim 1, wherein the cytokine cocktail comprises a fusion protein comprising an IL-12 moiety and an IL-18 moiety.

12. A method of generating cytokine induced memory-like NK-92 cells, the method comprising:
exposing NK-92 cells to a medium comprising a cytokine cocktail, wherein the cytokine cocktail comprises individual cytokines or fusion proteins comprised thereof, wherein the cocktail comprises IL-15 or N-803, IL-12, and IL-18;
removing the cytokine cocktail from the medium; and
administering a therapeutically effective amount of the NK-92 cells to the patient.

13. The method of claim 12, wherein the NK-92 cells are exposed to the cytokine cocktail for 12 hours, 24 hours, 36 hours, 48 hours, or 96 hours.

14. The method of claim 12, wherein the NK-92 cells are cryopreserved after removing the cytokine cocktail.

15. (canceled)

16. The method of claim 12, further comprising engineering the NK-92 cells to express a CAR prior to, during, or after the cytokine treatment.

17-22. (canceled)

23. A method of treating cancer in a subject in need thereof, comprising administering an effective amount of cytokine induced memory-like (CIML) NK-92 cells according to claim 1, wherein the CIML-like properties enhance anti-tumor activity, persistence, and memory recall of the administered cells in vivo.

24. The method of claim 23, wherein the CIML-like NK-92 cells are cryopreserved and provided as an off-the-shelf composition.

25. The method of claim 23, wherein target cells treated with supernatants from the CIML-like NK-92 cells exhibit increased PD-L1 expression and enhanced sensitivity to killing by CAR-engineered NK-92 cells.

26. The method of claim 23, wherein the CIML properties persist for at least 14 days post-cytokine stimulation.

27. The method of claim 23, wherein the NK-92 cells reduces immunosuppressive tumor-associated cells, including myeloid-derived suppressor cells (MDSCs).

27-55. (canceled)
